(19) United States
(12) Patent Application Publication
(10) Pub. No.: US 2018/0185408 A1
(43) Pub. Date: Jul. 5, 2018

(54) ANTIFUNGAL NYLON-3 POLYMERS AND COMBINATION THERAPY USING THEM TO TREAT FUNGAL INFECTIONS

(71) Applicant: Wisconsin Alumni Research Foundation, Madison, WI (US)

(72) Inventors: Runhui Liu, Shanghai (CN); Bernard Weisblum, Madison, WI (US); Samuel Gellman, Madison, WI (US); Fang Yun Lim, Madison, WI (US); Leslie Anne Rank, Madison, WI (US); Nancy P. Keller, Madison, WI (US); Jin Woo Bok, Madison, WI (US); Christina M. Hull, Middleton, WI (US); Naomi Marie Walsh, Madison, WI (US); Mingwei Huang, Madison, WI (US)

(21) Appl. No.: 15/850,416

(22) Filed: Dec. 21, 2017

Related U.S. Application Data
(60) Provisional application No. 62/437,151, filed on Dec. 21, 2016.

(51) Int. Cl. A61K 31/785; A61P 31/10

(52) U.S. Cl. CPC A61K 31/785; A61P 31/10

(57) ABSTRACT

A method and corresponding pharmaceutical composition for inhibiting the growth of fungi. The method uses an antifungal composition containing as the active ingredient one or more nylon-3 copolymers having a formula:

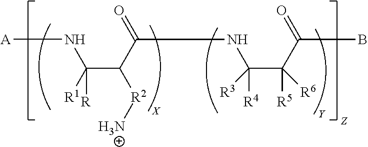

or a salt thereof,
   wherein:
each R is independently hydrogen or C₁-C₆-alkyl;
each R¹, R³, R⁴, R⁵, and R⁶ are each independently selected from the group consisting of hydrogen or substituted or unsubstituted C₁-C₆-alkyl;
each R² is C₁-C₆-alkylene;
“A” is hydrogen or an amino-protecting group;
“B” is hydroxyl or a carboxy-protecting group; and
“X,” “Y,” and “Z” are positive numbers.

### CROSS-REFERENCE TO RELATED APPLICATIONS

Priority is hereby claimed to provisional application Ser. No. 62/437,151, filed Dec. 21, 2016, which is incorporated herein by reference.

### FEDERAL FUNDING STATEMENT

This invention was made with government support under GM093265 and AI065728 awarded by the National Institutes of Health. The government has certain rights in the invention.

### FIELD OF THE INVENTION

Disclosed are nylon-3 copolymers that are broadly effective to inhibit the growth of fungi, while displaying only mild to moderate toxicity against mammalian host cells (including human host cells). Thus, the present disclosure is directed to these nylon-3 copolymers and their use, alone and in combination with other antifungal agents, to prevent and to inhibit fungal infections.

### BACKGROUND

Many naturally occurring, biologically active compounds are proteins or peptides based upon α-amino acids (i.e., sequences of α-amino acids in which the α-carboxyl group of one amino acid is joined by an amide bond to the α-amino group of the adjacent amino acid). In recent years an approach to the discovery of new pharmaceutically active drugs has been to synthesize libraries of peptides and then to assay for compounds within the library which have a desired activity, such as a desired binding activity. However, α-amino acid peptides are not altogether satisfactory for pharmaceutical uses, in particular because they are often poorly absorbed and subject to proteolytic degradation in vivo.

Much work on β-amino acids and peptides synthesized from ß-amino acids has been reported in the scientific and patent literature. See, for example, the work performed by a group led by current co-inventor Samuel H. Gellman, including: “Application of Microwave Irradiation to the Synthesis of 14-helical Beta-Peptides,” Murray & Gellman,” Organic Letters (2005) 7(8), 1517-1520; “Synthesis of 2,2-Disubstituted Pyrrolidine-4-carboxylic Acid Derivatives and Their Incorporation into Beta-Peptide Oligomers,” Huck & Gellman, J. Org. Chem. (2005) 70(9), 3353-62; “Effects of Conformational Stability and Geometry of Guanidinium Display on Cell Entry by Beta-Peptides,” Potocky, Menon, & Gellman, Journal of the American Chemical Society (2005) 127(11):3686-7; “Residue requirements for helical folding in short alpha/beta-peptides: crystallographic characterization of the 11-helix in an optimized sequence,” Schmitt, Choi, Guzei, & Gellman, Journal of the American Chemical Society (2005), 127(38), 13130-1 and “Efficient synthesis of a beta-peptide combinatorial library with microwave irradiation,” Murray, Farooqi, Sadowsky, Scalf, Freund, Smith, Chen, & Gellman, Journal of the American Chemical Society (2005), 127(38), 13271-80. Another group, led by Dieter Seebach in Zurich, Switzerland, has also published extensively in the beta-polypeptide field. See, for example, Seebach et al. (1996) Helv. Chim. Acta. 79:913-941; and Seebach et al. (1996) Helv. Chim. Acta. 79:2043-2066. In the first of these two papers Seebach et al. describe the synthesis and characterization of a β-hexapeptide, namely (H-β-HVal-β-HAla-β-HLeu) 2-OH. Interestingly, this paper specifically notes that prior art reports on the structure of β-peptides have been contradictory and “partially controversial.” In the second paper, Seebach et al. explore the secondary structure of the above-noted β-hexapeptide and the effects of residue variation on the secondary structure.

Dado and Gellman (1994) J. Am. Chem. Soc. 116:1054-1062 describe intramolecular hydrogen bonding in derivatives of β-alanine and γ-amino butyric acid. This paper postulates that β-peptides will fold in manners similar to α-amino acid polymers if intramolecular hydrogen bonding between nearest neighbor amide groups on the polymer backbone is not favored.

Suhara et al. (1996) Tetrahedron Lett. 37(10):1575-1578 report a polysaccharide analog of a β-peptide in which D-glycocylamine derivatives are linked to each other via a C-1 β-carboxylate and a C-2 α-amino group. This class of compounds has been given the trivial name “carbopeptoids.”

Regarding methods to generate combinatorial libraries, several reviews are available. See, for instance, Ellman (1996) Acc. Chem. Res. 29:132-143 and Lam et al. (1997) Chem. Rev. 97:411-448.

In the recent patent literature relating to ß-polypeptides, see, for example, U.S. published patent applications 2008/0166388, titled “Beta-Peptides with Antifungal Activity”; 2008/0058548, titled Concise Beta2-Amino Acid Synthesis via Organocatalytic Aminomethylation”; 2007/0154882, titled “Beta-polypeptides that inhibit cytomegalovirus infection”; 2007/0123709, titled “Beta-amino acids”; and 2007/0087404, titled “Poly-beta-peptides from functionalized beta-lactam monomers and antibacterial compositions containing same.” See also U.S. published patent application 2003/0212250, titled “Peptides.”

Invasive fungal disease in the US is associated with a mortality rate that often rises beyond 50 percent, emphasizing the need for improved treatment strategies. (Brown G D, Denning D W, Gow N A R, Levitz S M, Netea M G, White T C. 2012. Hidden killers: human fungal infections. Sci Transl Med 4:165rv13.) Current therapeutics are limited, and many antifungal drugs lack efficacy or are toxic to humans. (Butts A, Krysan D J. 2012. Antifungal Drug Discovery: Something Old and Something New. PLOS Pathog 8:e1002870. Roemer T, Krysan D J. 2014. Antifungal drug development: challenges, unmet clinical needs, and new approaches. Cold Spring Harb Perspect Med 4.) Furthermore, increasing antifungal drug resistance has been observed. (Polvi E J, Li X, O'Meara T R, Leach M D, Cowen L E. 2015. Opportunistic yeast pathogens: reservoirs, virulence mechanisms, and therapeutic strategies. Cell Mol Life Sci CMLS 72:2261-2287. Verweij P E, Snelders E, Kema G H J, Mellado E, Melchers W J G. 2009. Azole resistance in Aspergillus fumigatus: a side-effect of environmental fungicide use? Lancet Infect Dis 9:789-795. Sanguinetti M, Posteraro B, Lass-Flörl C. 2015. Antifungal drug resistance among Candida species: mechanisms and clinical impact. Mycoses 58 Suppl 2:2-13. Shah D N, Yau R, Lasco T M, Weston J, Salazar M, Palmer H R, Garey K W. 2012. Impact of Prior Inappropriate Fluconazole Dosing on Isolation of Fluconazole-Nonsusceptible Candida Species in Hospitalized Patients with Candidemia. Antimicrob Agents Chemother 56:3239-3243.) The need for the development of new antifungal drugs is obvious; however, only one new class of drugs (echinocandins), has been licensed within the past 15 years. (Ostrosky-Zeichner L, Casadevall A, Galgiani J N, Odds F C, Rex J H. 2010. An insight into the antifungal pipeline: selected new molecules and beyond. Nat Rev Drug Discov 9:719-727.) A major hurdle in antifungal development is that both fungi and humans are eukaryotes and similarities in processes, cell structures, and essential proteins leave a limited number of unique drug targets. (Monk B C, Cannon R D. 2002. Genomic pathways to antifungal discovery. Curr Drug Targets Infect Disord 2:309-329. Odds F C, Brown A J P, Gow N A R. 2003. Antifungal agents: mechanisms of action. Trends Microbiol 11:272-279.)

Naturally occurring host-defense peptides (HDPs) represent one of the first forms of chemical defense by eukaryotic cells against bacteria, fungi, viruses, and protozoa. (Zasloff M. 2002. Antimicrobial peptides of multicellular organisms. Nature 415:389-395. Mookherjee N, Hancock R E, W. 2007. Cationic host defence peptides: Innate immune regulatory peptides as a novel approach for treating infections. Cell Mol Life Sci 64:922-33.) HDPs have diverse amino acid compositions and sizes, though these peptides are usually cationic, amphipathic molecules. The targets of most HDPs remain unclear, though some mechanisms of antifungal HDPs have been reported. These include binding of HDPs to the cell wall, membrane permeabilization, and interactions with intercellular targets to generate reactive oxygen species, leading to apoptosis. Because HDPs are naturally produced by eukaryotic host cells, it is reasonable to conclude that they are likely low in toxicity to those same cells, while still targeting invading pathogens. This combination makes HDPs appealing platforms for drug development.

A variety of synthetic, cationic polymers resembling HDPs have been developed with antibacterial activity and relatively low toxicity towards eukaryotic cells (human red blood cells). (See, for example, Lienkamp K, Madkour A E, Musante A, Nelson C F, Niisslein K, Tew G N. 2008. Antimicrobial Polymers Prepared by ROMP with Unprecedented Selectivity: A Molecular Construction Kit Approach. J Am Chem Soc 130:9836-9843. Mowery B P, Lindner A H, Weisblum B, Stahl S S, Gellman S H. 2009. Structure—activity Relationships among Random Nylon-3 Copolymers That Mimic Antibacterial Host-Defense Peptides. J Am Chem Soc 131:9735-9745. Kuroda K, DeGrado W F. 2005. Amphiphilic Polymethacrylate Derivatives as Antimicrobial Agents. J Am Chem Soc 127:4128-4129. Palermo E F, Sovadinova I, Kuroda K. 2009. Structural Determinants of Antimicrobial Activity and Biocompatibility in Membrane-Disrupting Methacrylamide Random Copolymers. Biomacromolecules 10:3098-3107. Song A, Walker S G, Parker K A, Sampson N S. 2011. Antibacterial Studies of Cationic Polymers with Alternating, Random, and Uniform Backbones. ACS Chem Biol 6:590-599. Sellenet P H, Allison B, Applegate B M, Youngblood J P. 2007. Synergistic Activity of Hydrophilic Modification in Antibiotic Polymers. Biomacromolecules 8:19-23. Li P, Zhou C, Rayatpisheh S, Ye K, Poon Y F, Hammond P T, Duan H, Chan-Park M B. 2012. Cationic Peptidopolysaccharides Show Excellent Broad-Spectrum Antimicrobial Activities and High Selectivity. Adv Mater 24:4130-4137. Jiang Y, Yang X, Zhu R, Hu K, Lan W-W, Wu F, Yang L. 2013. Acid-Activated Antimicrobial Random Copolymers: A Mechanism-Guided Design of Antimicrobial Peptide Mimics. Macromolecules 46:3959-3964. Sambhy V, Peterson B R, Sen A. 2008. Antibacterial and Hemolytic Activities of Pyridinium Polymers as a Function of the Spatial Relationship between the Positive Charge and the Pendant Alkyl Tail. Angew Chem Int Ed 47:1250-1254.) Far fewer examples of synthetic polymers with selective activity against fungi and minimum cytotoxicity towards mammalian cells exist. (Chin W, Yang C, Ng V W L, Huang Y, Cheng J, Tong Y W, Coady D J, Fan W, Hedrick J L, Yang Y Y. 2013. Biodegradable Broad-Spectrum Antimicrobial Polycarbonates: Investigating the Role of Chemical Structure on Activity and Selectivity. Macromolecules 46:8797-8807. Michl T D, Locock K E S, Stevens N E, Hayball J D, Vasilev K, Postma A, Qu Y, Traven A, Haeussler M, Meagher L, Griesser H J. 2014. RAFT-derived antimicrobial polymethacrylates: elucidating the impact of end-groups on activity and cytotoxicity. Polym Chem 5:5813-5822. Liu R, Chen X, Hayouka Z, Chakraborty S, Falk S P, Weisblum B, Masters K S, Gellman S H. 2013. Nylon-3 Polymers with Selective Antifungal Activity. J Am Chem Soc 135:5270-5273. Liu R, Chen X, Falk S P, Mowery B P, Karlsson A J, Weisblum B, Palecek S P, Masters K S, Gellman S H. 2014. Structure—Activity Relationships among Antifungal Nylon-3 Polymers: Identification of Materials Active against Drug-Resistant Strains of Candida albicans. J Am Chem Soc 136:4333-4342.) The reason for this disparity is the result of fundamental differences between prokaryotic and eukaryotic cellular membranes that facilitate targeting bacterial cell membranes via mechanisms that do not damage eukaryotic host cells.

The antifungal activity of nylon-3 polymers has been directly correlated with high hemolytic activity, lacking selectivity for fungal cells relative to that of mammalian cells. (Carlton A J, Pomerantz W C, Weisblum B, Gellman S H, Palecek S P. 2006. Antifungal Activity from 14-Helical β-Peptides. J Am Chem Soc 128:12630-12631. Chongsiriwatana N P, Miller T M, Wetzler M, Vakulenko S, Karlsson A J, Palecek S P, Mobashery S, Barron A E. 2011. Short Alkylated Peptoid Mimics of Antimicrobial Lipopeptides. Antimicrob Agents Chemother 55:417-420.) Newer research has revealed a family of cationic nylon-3 homopolymers that was found to display both a strong and selective antifungal activity profile against the K1 clinical isolate of C. albicans. However, introducing hydrophobic subunits into the homopolymers did not increase the antifungal profile of the compounds against C. albicans, but rather served only to increase the hemolytic activity of these nylon-3 polymers. (Liu R, Chen X, Hayouka Z, Chakraborty S, Falk S P, Weisblum B, Masters K S, Gellman S H. 2013. Nylon-3 Polymers with Selective Antifungal Activity. J Am Chem Soc 135:5270-5273. Liu R, Chen X, Falk S P, Mowery B P, Karlsson A J, Weisblum B, Palecek S P, Masters K S, Gellman S H. 2014. Structure—Activity Relationships among Antifungal Nylon-3 Polymers: Identification of Materials Active against Drug-Resistant Strains of Candida albicans. J Am Chem Soc 136:4333-4342.)

### SUMMARY OF THE INVENTION

Nylon-3 polymers, or polydisperse β-peptides, have great potential as antimicrobial polymers due to their backbone similarity to natural α-peptides and fast, cost-effective synthesis. As disclosed herein, the nylon-3 polymers are synthesized by anionic ring-opening polymerization (AROP) of β-lactams to generate sequence random polymers with narrow molecular weight distributions. The nylon-3 backbone is analogous to that of natural α-peptides; however, the space between amide bonds is extended by one extra carbon atom. The nylon-3 polymers disclosed herein are biocompatible with mammalian systems, are generally stable under physiological conditions, and exhibit minimal toxicity towards eukaryotic cells.

Thus, disclosed herein is a method of inhibiting fungal growth. The method comprises contacting fungi with a composition comprising a nylon-3 copolymer having a formula:

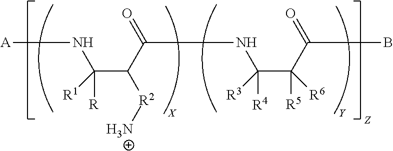

or a salt thereof,

wherein:

each R is independently hydrogen or substituted or unsubstituted C₁-C₆-alkyl;

R¹, R³, R⁴, R⁵, and R⁶ are each independently selected from the group consisting of hydrogen and substituted or unsubstituted C₁-C₆-alkyl;

Each R² is independently C₁-C₆-alkylene;

“A” is hydrogen or an amino-protecting group;

“B” is hydroxyl or a carboxy-protecting group; and

“X,” “Y,” and “Z” are positive numbers.

The nylon-3 copolymer may be a random copolymer or a block copolymer.

In a preferred version of the compound, R is hydrogen and the substituents R¹, R³, R⁴, R⁵, and R⁶ may each be methyl and R² may be methylene. The substituent “A” may be

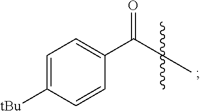 [SMILES: *C(=O)c1ccc(C(C)(C)C)cc1]

and
the substituent “B” may be

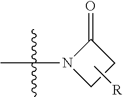

wherein R is hydrogen or C₁-C₆-alkyl.

The subscript “X” may be a number between 0.1 and 0.9, “Y” may be a number between 0.1 and 0.9, and “Z” may be a number between 5 and 100. Alternatively, “X” may be a number between 0.1 and 0.9, “Y” may be a number between 0.1 and 0.9, and “Z” may be a number between 10 and 50. In yet another version, “X” may be a number between 0.1 and 0.9, “Y” may be a number between 0.1 and 0.9, and “Z” may be a number between 10 and 20.

The composition works on contact to inhibit the growth of at least fungi of the genera Aspergillus, Candida, Cryptococcus, and/or Fusarium.

Also disclosed herein is a method of inhibiting fungal infections in mammals. The method comprises administering to a mammalian subject in need thereof a fungal growth-inhibiting amount of a nylon-3 copolymer having a formula:

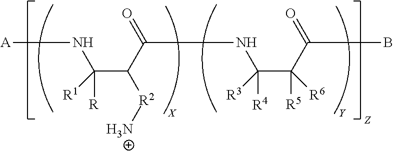

or a salt thereof,

wherein:

each R is independently hydrogen or substituted or unsubstituted C₁-C₆-alkyl;

R¹, R³, R⁴, R⁵, and R⁶ are each independently selected from the group consisting of hydrogen and substituted or unsubstituted C₁-C₆-alkyl;

each R² is C₁-C₆-alkylene;

“A” is hydrogen or an amino-protecting group;

“B” is hydroxyl or a carboxy-protecting group; and

“X,” “Y,” and “Z” are positive numbers.

Also disclosed herein is a pharmaceutical composition comprising:

a fungal growth-inhibiting amount of a nylon-3 copolymer having a formula:

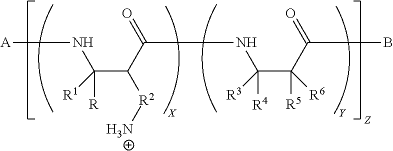

or a pharmaceutically suitable salt thereof,

wherein:

each R is independently hydrogen or substituted or unsubstituted C₁-C₆-alkyl;

R¹, R³, R⁴, R⁵, and R⁶ are each independently selected from the group consisting of hydrogen and substituted or unsubstituted C₁-C₆-alkyl;

each R² is C₁-C₆-alkylene;

“A” is hydrogen or an amino-protecting group;

“B” is hydroxyl or a carboxy-protecting group; and

“X,” “Y,” and “Z” are positive numbers;

in combination with a pharmaceutically suitable carrier.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, 5, 6, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made. The indefinite article “a,” when applied to a claimed element, means “one or more,” unless explicitly stated to the contrary.

All combinations of method or process steps as used herein can be performed in any order, unless otherwise specified or clearly implied to the contrary by the context in which the referenced combination is made.

The methods, compounds, and compositions of the present invention can comprise, consist of, or consist essentially of the essential elements and limitations of the invention as described herein, as well as any additional or optional ingredients, components, or limitations described herein or otherwise useful in synthetic organic chemistry.

### BRIEF DESCRIPTION OF THE DRAWINGS

FIG. 1. Nylon-3 copolymers are synthesized by the anionic ring opening polymerization (AROP) of racemic β-Lactams. The MM:TM copolymer was synthesized by the AROP of a cationic subunit and a hydrophobic subunit. βMM (monomethyl) and βTM (tetramethyl) were mixed in a 2:3 molar ratio with an intended average chain length of 20. Five mol % 4-tert-butylbenzoyl chloride was used as the reaction initiator in the presence of LiN(TMS)₂ (lithium bis(trimethylsilyl)amide). Deprotection was carried out with TFA (trifluoroacetic acid) at room temperature. Due to differences in chemical reactivity of the β-lactams, the resulting MM:TM copolymers were roughly 80 mol % MM and 20 mol % TM, with a 15-mer average chain length (MW=3300 g/mol).

FIGS. 2A and 2B. Toxicity of MM:TM to host cells compared to currently used antifungals. Fibroblasts (NIH 3T3), macrophages (RAW 264.7) and red blood cells (primary, human), were tested in dilution series of concentrations of MM:TM, amphotericin B, and fluconazole. The percentage of cell death for each compound was compared to no treatment (0% death) and complete lysis (100% death) controls. Solid horizontal lines represent the highest concentrations tested in each dilution series (121 μM for MM:TM, 216 μM for AMB, and 2612 μM for FLC). FIG. 2A: The concentration of drug necessary to cause lysis of 10% of macrophages/fibroblasts (IC₁₀) or hemolysis of 10% of hRBCs (HC₁₀). FIG. 2B: The concentration of drug necessary to cause lysis of 50% of macrophages/fibroblasts (IC₅₀) or hemolysis of 50% of hRBCs (HC₅₀). In cases where toxicity values landed between two concentrations, the lower concentration is reported. *RBCs were not tested with fluconazole. MM:TM=nylon-3 copolymer, AMB=amphotericin B, FLC=fluconazole.

FIG. 3. A graph depicting percent hemolysis for MM:TM 40:60 as a function of concentration.

FIGS. 4A and 4B. Effect of copolymer on Arabadopsis thaliana root growth. Root growth (in cm) was determined 6 and 10 days after germination of seeds in 0, 1, 10, and 100 μg/mL polymer. FIG. 4A: Representative images of roots after 10 days of growth. Blue dashed lines mark growth at day 6. FIG. 4B: Quantitation of average root growth (in cm) between days 6 and 10 for each category. Data represents averages of a minimum of 37 replicate, error bars indicate standard error. Treatment groups were compared to the control for significance using a student's t-test. N.S. indicates p>0.01, ***p=1.94e-19.

### DETAILED DESCRIPTION

In the present description unless otherwise indicated terms such as “compounds of the invention” and “compounds disclosed herein” embrace the compounds in salt form as well as in free base form and also when the compounds are attached to a solid phase. Where a basic substituent such as an amine substituent is present, the salt form may be an acid addition salt, for example a dihydrochloride. Salts include, without limitation, those derived from mineral acids and organic acids, explicitly including hydrohalides, e.g., hydrochlorides and hydrobromides, sulfates, phosphates, nitrates, sulfamates, acetates, citrates, lactates, tartrates, malonates, oxalates, salicylates, propionates, succinates, fumarates, maleates, methylene bis-b-hydroxynaphthoates, gentisates, isethionates, di-p-toluoyltartrates, methane sulfonates, ethanesulfonates, benzenesulfonates, p-toluenesulfonates, cyclohexylsulfamates, quinates, and the like. Base addition salts include those derived from alkali or alkaline earth metal bases or conventional organic bases, such as triethylamine, pyridine, piperidine, morpholine, N methylmorpholine, and the like. Other suitable salts are found in, for example, “Handbook of Pharmaceutical Salts: Properties, Selection, and Use, 2^{nd} Revised Edition,” P. H. Stahl and C. G. Wermuch, Eds., © 2011 Wiley-VCH, an imprint of John Wiley & Sons, Inc. (Hoboken, N.J.), ISBN 978-3-90639-051-2, which is incorporated herein by reference.

The nylon-3 residues disclosed herein are characteristically β-amino-n-propionic acid derivatives, typically further substituted at the 2-position carbon atom (the β² carbon) and/or the 3-position carbon atom (the β³ carbon) in the backbone and may be further substituted, e.g., at the N-terminal amino nitrogen atom. The β², β³, and amino substituents may include substituents containing from 1 to 43 carbon atoms optionally interrupted by up to 4 hetero atoms, selected from O, N or S, optionally containing a carbonyl (i.e., —C(O)—) group, and optionally further substituted by up to 6 substituents selected from halo, NO₂, —OH, C₁₋₄ alkyl, —SH, —SO₃, —NH₂, C₁₋₄-acyl, C₁₋₄-acyloxy, C₁₋₄-alkylamino, C₁₋₄-dialkylamino, trihalomethyl, —CN, C₁₋₄-alkylthio, C₁₋₄-alkylsulfinyl, or C₁₋₄-alkylsulfonyl.

Substituents on the β² and/or β³ carbon atoms of β-amino acid residues may be selected from the group comprising the substituents which are present on the α-carbon atoms of natural α-amino acids, e.g., —H, —CH³, —CH(CH₃)₂, —CH₂—CH(CH₃)₂, CH(CH₃)CH₂CH₃, —CH₂-phenyl, CH₂-pOH-phenyl, —CH₂-indole, —CH₂—SH, —CH₂—CH₂—S—CH₃, —CH₂OH, —CHOH—CH₃, —CH₂—CH₂—CH₂—CH₂—NH₂, —CH₂—CH₂—CH₂—NH—C(NH)NH₂, —CH₂-imidazole, —CH—COOH, —CH₂—CH₂—COOH, —CH₂—CONH₂, —CH₂—CH₂—CONH₂ or together with an adjacent NH group defines a pyrrolidine ring, as is found in the proteinogenic α-amino acid proline.

The term “substituted” indicates that one or more hydrogen atoms on the group indicated in the expression using “substituted” is replaced with a “substituent”. The substituent can be one of a selection of indicated groups, or it can be a suitable group known to those of skill in the art, provided that the substituted atom's normal valency is not exceeded, and that the substitution results in a stable compound. Suitable substituent groups include, e.g., alkyl, alkenyl, alkynyl, alkoxy, halo, haloalkyl, hydroxy, hydroxyalkyl, aryl, aroyl, (aryl)alkyl (e.g., benzyl or phenylethyl), heteroaryl, heterocycle, cycloalkyl, alkanoyl, alkoxycarbonyl, amino, alkylamino, dialkylamino, trifluoromethyl, trifluoromethoxy, trifluoromethylthio, difluoromethyl, acylamino, nitro, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl(alkyl)amine, and cyano. Additionally, suitable substituent groups can be, e.g., —X, —R, —O—, —OR, —SR, —S—, —NR₂, —NR₃, ═NR, —CX₃, —CN, —OCN, —SCN, —N═C═O, —NCS, —NO, —NO2, ═N₂, —N₃, —NC(═O)R, —C(═O)R, —C(═O)NRR, —S(═O)₂O—, —S(═O)₂OH, —S(═O)₂R, —OS(═O)₂OR, —S(═O)₂NR, —S(═O)R, —OP(═O)O₂RR, P(═O)O₂RR, —P(═O)(O—)₂, —P(═O)(OH)₂, —C(═O)R, —C(═O)X, —C(S)R, C(O)OR, —C(O)O—, —C(S)OR, —C(O)SR, —C(S)SR, —C(O)NRR, —C(S)NRR, or —C(NR)NRR, where each X is independently a halogen (“halo”): F, Cl, Br, or I; and each R is independently H, alkyl, aryl, (aryl)alkyl (e.g., benzyl), heteroaryl, (heteroaryl)alkyl, heterocycle, heterocycle(alkyl), or a protecting group. As would be readily understood by one skilled in the art, when a substituent is keto (═O) or thioxo (═S), or the like, then two hydrogen atoms on the substituted atom are replaced. In some embodiments, one or more of the substituents above are excluded from the group of potential values for substituents on the substituted group.

The invention includes the compounds of the invention in pure isomeric form, e.g., consisting of at least 90%, preferably at least 95% of a single isomeric form, as well as mixtures of these forms. The compounds of the invention may also be in the form of individual enantiomers or may be in the form of racemates or diastereoisomeric mixtures or any other mixture of the possible isomers.

The compounds of the invention may be prepared by the synthetic chemical procedures described herein, as well as other procedures similar to those which may be used for making α-amino acid peptides. Such procedures include both solution and solid phase procedures, e.g., using both Boc and Fmoc methodologies. Thus, the compounds described herein may be prepared by successive amide bond-forming procedures in which amide bonds are formed between the β-amino group of a first β-amino acid residue or a precursor thereof and the α-carboxyl group of a second β-amino acid residue or α-amino acid residue or a precursor thereof. The amide bond-forming step may be repeated as many times, and with specific α-amino acid residues and/or β-amino acid residues and/or precursors thereof, as required to give the desired α/β-polypeptide. Also, peptides comprising two, three, or more amino acid residues (α or β) may be joined together to yield larger α/β-peptides. Cyclic compounds may be prepared by forming peptide bonds between the N-terminal and C-terminal ends of a previously synthesized linear polypeptide.

β³-amino acids may be produced enantioselectively from corresponding α-amino acids; for instance, by Arndt-Eisert homologation of N-protected α-amino acids. Conveniently such homologation may be followed by coupling of the reactive diazo ketone intermediate of the Wolff rearrangement with a β-amino acid residue.

The method described herein can be used to establish discrete compound collections or libraries of compounds for use in screening for compounds having desirable activities, in particular biological activities indicative of particular pharmaceutical uses.

Thus, the invention also includes discrete compound collections (typically comprising from 2 to about 1000 compounds) and libraries of compounds (typically comprising from 20 to 100 compounds up to many thousands of compounds, e.g., 100,000 compounds or more) comprising pluralities of the compounds described herein.

Thus, the invention includes compounds as described herein for use as pharmaceuticals and the use of the compounds for the manufacture of a medicament for the treatment of any disease associated with any of the assays described herein, including infection or colonization by fungi of any description. The invention also includes the use of a compound fabricated according to the claimed method as a pharmaceutical, and pharmaceutical compositions comprising an effective amount of such a compound together with a pharmaceutically acceptable diluent or carrier. The compositions can also be used to treat agricultural fungal infestations.

By way of shorthand, several specific compounds within the scope of this disclosure were tested for the fungal growth inhibiting properties:

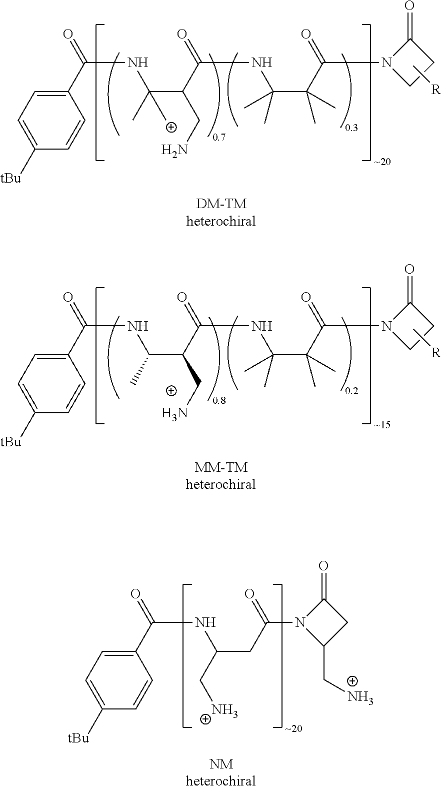

To determine the breadth of nylon-3 polymer activity, the antifungal qualities of MM-TM, DM-TM, and NM were evaluated against a diverse array of both filamentous and non-filamentous pathogenic fungi across the fungal kingdom, including members of the Zygomycetes, Ascomyetes, and Basidiomycetes phyla. Surprisingly, most fungi tested, including those naturally resistant to current antifungal drugs, were sensitive to the nylon-3 polymers. Thus, the nylon-3 polymers are useful against fungi for which there are only limited and often ineffective therapeutic agents available at present.

As used herein, the terms “fungus” and its plural “fungi” are defined broadly to encompass any and all organisms falling with the Kingdom “Fungi.” While the taxonomic definitions are somewhat fluid and subject to revision, the terms “fungus” and “fungi” include

### Phylum Chytridiomycota

Class Neocallimastigomycetes

Class Hyaloraphidiomycetes

Class Monoblepharidomycetes

Class Chytridiomycetes

### Phylum Blastocladiomycota

Class Blastocladiomycetes

### Phylum Olpidiomycota

Class Olpidiomycetes

### Phylum Entomophthoromycota

Class Neozygitomycetes

Class Basidiobolomycetes

Class Entomophthoromycetes

### Phylum Kickxellomycota

Class Zoopagomycetes

Class Kickxellomycetes

### Phylum Mucoromycota

Class Mortierellomycetes

Class Mucoromycetes

### Phylum Glomeromycota

Class Glomeromycetes

### Phylum Entorrhizomycota

Class Entorrhizomycetes

### Phylum Basidiomycota

Subphylum Pucciniomycotina
Class Tritirachiomycetes
Class Mixiomycetes
Class Agaricostilbomycetes
Class Cystobasidiomycetes
Class Microbotryomycetes
Class Classiculomycetes
Class Cryptomycocolacomycetes
Class Atractiellomycetes
Class Pucciniomycetes

Subphylum Ustilaginomycotina
Class Monilielliomycetes
Class Malasseziomycetes
Class Ustilaginomycetes
Class Exobasidiomycetes

Subphylum Agaricomycotina
Class Wallemiomycetes
Class Bartheletiomycetes
Class Tremellomycetes
Class Dacrymycetes
Class Agaricomycetes
   Subclass Phallomycetidae
   Subclass Agaricomycetidae

### Phylum Ascomycota

Subphylum Taphrinomycotina
Class Archaeorhizomycetes
Class Neolectomycetes
Class Pneumocystidomycetes
Class Schizosaccharomycetes
Class Taphrinomycetes

Subphylum Saccharomycotina
Class Saccharomycetes

Subphylum Pezizomycotina
Class Orbiliomycetes
Class Pezizomycetes
Class Coniocybomycetes
Class Arthoniomycetes
Class Dothideomycetes
   Subclass Dothideomycetidae
   Subclass Pleosporomycetidae
Class Eurotiomycetes
   Subclass Mycocaliciomycetidae
   Subclass Chaetothyriomycetidae
   Subclass Eurotiomycetidae
Class Lichinomycetes
Class Lecanoromycetes
   Subclass Acarosporomycetidae
   Subclass Ostropomycetidae
   Subclass Lecanoromycetidae
Class Xylonomycetes
Class Geoglossomycetes
Class Leotiomycetes
Class Laboulbeniomycetes
Class Sordariomycetes
   Subclass Meliolomycetidae
   Subclass Xylariomycetidae
   Subclass Hypocreomycetidae
   Subclass Sordariomycetidae

### See Esser, Karl (2014). “The Mycota VII A: Systematics and Evolution (2nd ed.),” Springer: 461. ISBN 978-3-642-55317-2.

A “protecting group” is any chemical moiety capable of selective addition to and removal from a reactive site to allow manipulation of a chemical entity at sites other than the reactive site. Many protecting groups are known in the art. A large number of protecting groups and corresponding chemical cleavage reactions are described in “Greene's Protective Groups in Organic Synthesis,” ISBN-13: 978-1118057483, ©2014, John Wiley & Sons, Inc. Greene describes many nitrogen protecting groups, for example, amide-forming groups. In particular, see Chapter 1, Protecting Groups: An Overview, Chapter 2, Hydroxyl Protecting Groups, Chapter 4, Carboxyl Protecting Groups, and Chapter 5, Carbonyl Protecting Groups. For additional information on protecting groups, see also Kocienski, Philip J. “Protecting Groups,” (Georg Thieme Verlag Stuttgart, New York, 1994), which is incorporated herein by reference. Typical nitrogen protecting groups described in Greene include benzyl ethers, silyl ethers, esters including sulfonic acid esters, carbonates, sulfates, and sulfonates. For example, suitable nitrogen protecting groups include substituted methyl ethers; substituted ethyl ethers; p-chlorophenyl, p-methoxyphenyl, 2,4-dinitrophenyl, benzyl; substituted benzyl ethers (p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, diphenylmethyl, 5-dibenzosuberyl, triphenylmethyl, p-methoxyphenyl-diphenylmethyl, di(p-methoxyphenyl)phenylmethyl, tri(p-methoxyphenyl)methyl, 1,3-benzodithiolan-2-yl, benzisothiazolyl S,S-dioxido); silyl ethers (silyloxy groups) (trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, t-butylmethoxy-phenylsilyl); esters (formate, benzoylformate, acetate, choroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), pivaloate, adamantoate, crotonate, 4-methoxycrotonate, benzoate, p-phenylbenzoate, 2,4,6-trimethylbenzoate(mesitoate)); carbonates (methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate); groups with assisted cleavage (2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate, 4-(methylthiomethoxy)butyrate, miscellaneous esters (2,6-dichloro-4-methylphenoxyacetate, 2,6-dichloro-4-(1,1,3,3 tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate (tigloate), o-(methoxycarbonyl)benzoate, p-poly-benzoate, α-naphthoate, nitrate, alkyl N,N,N′,N′-tetramethyl-phosphorodiamidate, n-phenylcarbamate, borate, 2,4-dinitrophenylsulfenate); or sulfonates (methanesulfonate(mesylate), benzenesulfonate, benzylsulfonate, tosylate, or triflate).

The more common of the amine-protecting groups have trivial abbreviations that are widely used in the literature and include: carbobenzyloxy (Cbz) group (removed by hydrogenolysis), p-methoxybenzyl carbonyl (Moz or MeOZ) group (removed by hydrogenolysis), tert-butyloxycarbonyl (BOC) group (common in solid phase peptide synthesis; removed by concentrated strong acid (such as HCl or CF₃COOH), or by heating to >80° C., 9-fluorenylmethyloxycarbonyl (FMOC) group (also common in solid phase peptide synthesis; removed by base, such as piperidine), acetyl (Ac) group (removed by treatment with a base), benzoyl (Bz) group (removed by treatment with a base), benzyl (Bn) group (removed by hydrogenolysis), carbamate group (removed by acid and mild heating), p-methoxybenzyl (PMB) (removed by hydrogenolysis), 3,4-dimethoxybenzyl (DMPM) (removed by hydrogenolysis), p-methoxyphenyl (PMP) group (removed by ammonium cerium(IV) nitrate (CAN)), tosyl (Ts) group (removed by concentrated acid and strong reducing agents), sulfonamide groups (Nosyl & Nps; removed by samarium iodide, tributyltin hydride.

The compounds of the invention may be synthesized using solid phase synthesis techniques.

Thus Fmoc-N-Protected β-amino acids can be used to synthesize poly-α/β-peptides by conventional manual solid-phase synthesis procedures under standard conditions on ortho-chloro-trityl chloride resin.

Esterification of Fmoc-β-amino acids with the ortho-chloro-trityl resin can be performed according to the method of Barlos et al., Tetrahedron Lett. (1989), 30, 3943. The resin (150 mg, 1.05 mmol Cl) is swelled in 2 ml CH₂Cl₂ for 10 min. A solution of the Fmoc-protected β-amino acid in CH₂Cl₂ and iPr₂EtN are then added successively and the suspension is mixed under argon for 4 h. Subsequently, the resin is filtered and washed with CH₂Cl₂/MeOH/iPr₂EtN (17:2:1, 3×3 min), CH₂Cl₂ (3×3 min), DMF (2×3 min), CH₂Cl₂ (3×3 min), and MeOH (2×3 min). The substitution of the resin is determined on a 3 mg sample by measuring the absorbance of the dibenzofulvene adduct at 300 nm. The Fmoc group is removed using 20% piperidine in DMF (4 ml, 2×20 min) under Ar bubbling. The resin is then filtered and washed with DMF (6×3 min). For each coupling step, a solution of the β-amino acid (3 equiv.), BOP (3 equiv.) and HOBT (3 equiv.) in DMF (2 ml) and iPr₂EtN (9 eq) are added successively to the resin and the suspension is mixed for 1 h under Ar. Monitoring of the coupling reaction is performed with 2,4,6-trinitrobenzene-sulfonic acid (TNBS) (W. S. Hancock and J. E. Battersby, Anal. Biochem. (1976), 71, 260). In the case of a positive TNBS test (indicating incomplete coupling), the suspension is allowed to react for a further 1 h. The resin is then filtered and washed with DMF (3×3 min) prior to the following Fmoc deprotection step. After the removal of the last Fmoc protecting group, the resin is washed with DMF (6×3 min), CH₂Cl₂ (3×3 min), Et₂O (3×3 min) and dried under vacuum for 3 h. Finally, the peptides are cleaved from the resin using 2% TFA in CH₂Cl₂ (2 ml, 5×15 min) under Ar. The solvent is removed and the oily residues are triturated in ether to give the crude α/ß-polypeptides. The compounds are further purified by HPLC.

The oral bioavailability of the compounds described herein is determined in the rat using standard procedures.

In view of the stable structures which α/β-peptides exhibit in solution, their stability to enzymatic degradation and their encouraging pharmacokinetic properties, the compounds of the invention have the potential to provide useful pharmaceutical products.

### Nylon-3 Copolymer MM:TM:

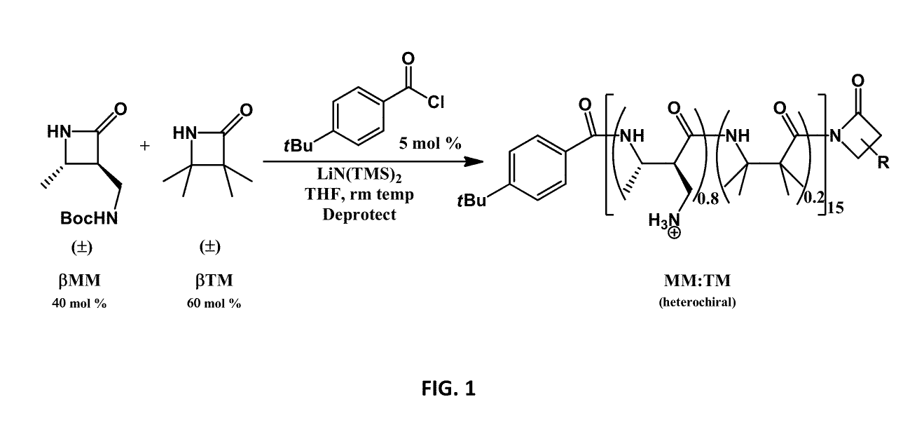

MM:TM copolymers were synthesized by the AROP of the β-Lactam subunits βMM (monomethyl) and βTM (tetramethyl) in a 2:3 cationic:hydrophobic molar ratio. The reaction scheme is shown in FIG. 1. 4-tert-butylbenzoyl chloride was used as the initiator in a 1:20 molar ratio to generate copolymers with an average 20-mer chain length. Because nylon-3 polymers are synthesized via a living polymerization, they have narrow molecular weight distributions. (Zhang J, Kissounko D A, Lee S E, Gellman S H, Stahl S S. 2009. Access to Poly-β-Peptides with Functionalized Side Chains and End Groups via Controlled Ring-Opening Polymerization of β-Lactams. J Am Chem Soc 131:1589-1597.) Narrow molecular weight distributions are important for designing molecules with specific activity profiles, because polymer species with different molecular weights are expected to display distinct pharmacological profiles in vivo. Polymer composition was analyzed by gel permeation chromatography (GPC) of protected nylon-3 copolymers and ¹H nuclear magnetic resonance (NMR) of deprotected nylon-3 copolymers. Normalizing the integration of aromatic protons from the initiator of each copolymer to represent one polymer chain length, the average monomer subunit composition was determined to be 4:1 MM:TM. The average chain length of the MM:TM copolymer was determined via NMR to be 15, with an average molecular weight of 3300 g/mol (Table 1). This average molecular weight was consequently used to calculate the molarity of all polymer solutions. We were able to produce MM:TM copolymer consistently with very little batch to batch variation in overall monomer subunit ratios, chain length, or molecular weight. As seen in Table 1, the degree of polymerization for each copolymer batch was determined by two different methods, GPC and NMR, with the chains length calculated by GPC consistently longer than that calculated via NMR. The discrepancy in chain lengths calculated between different methods is easily explained by the assumption used when calculating Dp_{GPC}. When calculating Dp_{GPC} from Mn_{GPC}, it is assumed that the desired monomer subunit ratio is achieved. Presuming that the MM:TM polymer has a 40:60 subunit ratio would skew calculated chain lengths to larger values, as the lower molecular weight monomer, TM, would be weighted to a larger degree in comparison to the heavier MM monomer. Therefore, the average Dp_{NMR} was selected for average molecular weight for molarity calculations because this calculated chain length accounts for the skewed incorporation of MM to TM monomers in a 4:1 ratio.

TABLE 1
MM:TM nylon-3 copolymer characterization.

| | | | | | NMR | | |
|---|---|---|---|---|---|---|---|
| | | | | | characterization | | |
| | | | | | Observed | | |
| | | | | | subunit | | |
| | | | | | ratio | | Av- |
| | | | | | (cationic: | | erage |
| | GPC characterization^{a,b} | | | | hydro- | | Bulk |
| Batch | PDI_{GPC}^{c} | Mn_{GPC}^{d} | Dp_{GPC}^{e} | Dp_{NMR}^{f} | phobic)^{g} | Mn_{NMR} | MW |
| 1^{a} | 1.27 | 4461 | 27 | 15 | 80:20 | 3280 | 3300 |
| 2^{a} | 1.18 | 4529 | 27 | 14 | 79:21 | 3058 | g/mol |
| 3^{a} | 1.20 | 3723 | 22 | 14 | 79:21 | 3723 | |
| 4^{a} | 1.19 | 4715 | 27 | 16 | 81:19 | 3504 | |
| 5^{b} | 1.24 | 2195 | 13^{a} | 12 | 75:25 | 2596 | |
| 6^{a} | 1.16 | 4262 | 21 | 15 | 80:20 | 3280 | |
| 7^{a} | 1.21 | 4168 | 25 | 15 | 80:20 | 3280 | |
| 8^{a} | 1.21 | 3291 | 19 | 13 | 77:33 | 3824 | |

^{a}Side-chain protected polymer characterization by gel permeation chromatography (GPC) using N,N-dimethylacetamide (DMAc) as the mobile phase.
^{b}Side-chain protected polymer characterization by GPC using tetrahydrofuran (THF) as the mobile phase.
^{c}Polydispersity Index.
^{d}The number average molecular weight of side-chain protected polymers.
^{e}The degree of polymerization, or average polymer chain length, as calculated from Mn_{GPC}.
^{f}The degree of polymerization, or average polymer chain length, as calculated by Nuclear Magnetic Resonance (NMR) integrations based on end group analysis of the tert-butyl benzoyl aromatic protons, setting the tert-butyl benzoyl aromatic protons to an integration of one polymer chain.
^{g}The subunit ratio of cationic to hydrophobic monomer incorporation for a particular polymer was calculated from NMR integrations, setting the tert-butyl benzoyl aromatic protons to an integration representative of one polymer chain.

### MM:TM Copolymer Shows Activity Against Multiple Species of Candida:

The ability to synthesize the MM:TM copolymer in a reproducible manner provided the impetus to test its antimicrobial/antifungal activity. Given the need for improved antifungal therapeutics, we used the CLSI M27-A3 broth microdilution method to determine the minimum inhibitory concentrations (MICs) of the MM:TM copolymer against four strains of Candida albicans and one strain of Candida lusitaniae. (See Table 5 in the Examples section). (M27-A3: “Reference Method for Broth Dilution Antifungal Susceptibility Testing of Yeasts,” © 2008, Clinical and Laboratory Standards Institute, Wayne, Pa., ISBN 1-56238-666-2.) Three of the four strains of C. albicans tested were drug resistant isolates: K1 (fluconazole resistant), Gu5 (fluconazole resistant), and E4 (fluconazole and amphotericin B resistant). MIC assays were carried out using all Candida strains at concentrations of 1.25×10³ and 1.25×10⁵ cells/mL at both 30° C. and 35° C. Both concentrations and temperatures provided consistent results in all cases (data not shown). The MICs of the MM:TM copolymer against all four strains of C. albicans were moderate around 5-9 μM for each (Table 2). Activity of the copolymer was strongest against C. lusitaniae (CL3), reaching average MIC concentrations of 3.8 μM. In all cases the MICs were equal or within one dilution of the Minimum Fungicidal Concentrations (MFCs), indicating that MM:TM is fungicidal against these Candida species (Tables 10 and 11, below). For comparison of MM:TM activity to currently used antifungal drugs, MIC and MFC values for the Candida test strains were determined using amphotericin B (AMB) and fluconazole (FLC), and all were in the expected range based on previous reports: sub-μM for AMB, except for the resistant strain E4, and low μM or totally resistant (>653.6 μM) to FLC for sensitive and resistant strains, respectively (Table 2 and Table 7, below). Interestingly, the AMB and FLC resistant strains of C. albicans showed no differences in sensitivity to the copolymer relative to sensitive strains suggesting that MM:TM may act via different mechanisms than both AMB and FLC.

TABLE 2
MIC and synergy data for MM:TM against Candida spp.
MIC Results by Broth Microdilution (μM)^{a,b}
Inoculum concentration of 1.25 × 10³ cells/mL at 35° C.

| | MM:TM^{c} | AMB^{d} | FLC^{e} |
|---|---|---|---|
| Isolate | MIC (μM) | MIC (μM) | MIC (μM) |
| C. albicans | 4.8 | 0.9 | >653.6 |
| (SC5314) | | | |
| C. lusitaniae | 1.4 | 0.4 | >653.6 |
| (CL3) | | | |
| C. albicans | 7.6 | 1.7 | >653.6 |
| (K1) | | | |
| C. albicans | 9.5 | 0.9 | >653.6 |
| (Gu5) | | | |
| C. albicans | 4.2 | 3.4 | >653.6 |
| (E4) | | | |

Synergy Results with FLC and MM:TM^{b}
Inoculum concentration of 1.25 × 10⁵ cells/mL at 30° C.

| | | MIC (μM) | | ΣFIC | FIC |
|---|---|---|---|---|---|
| Isolate | Test agent | Alone^{f} | Combined^{g} | Index^{h} | Interpret. |
| SC5314 | MM:TM | 20.2 | <0.02ⁱ | 1.00 | Indifferent |
| | FLC | <1.0ⁱ | <1.0 | | |
| K1 | MM:TM | 30.3 | 2.6 | 0.08 | Synergistic |
| | FLC | >163.5ⁱ | <1.3ⁱ | | |

^{a}MIC, minimum inhibitory concentration
^{b}Each experiment was repeated in duplicate on separate days in at least two different trial experiments
^{c}Average molecular weight of MM:TM copolymer used for molarity conversion is 3300 g/mol
^{d}AMB, amphotericin B (MW = 924.091 g/mol);
^{e}FLC, fluconazole (MW = 306.271 g/mol)
^{f}MIC as determined by OD₆₀₀ measurements after 48 hours for compound as a single agent
^{g}MIC as determined by OD₆₀₀ measurements after 48 hours for compounds incubated with Candida in combination.
^{h}Fractional inhibitory concentration (FIC)
ⁱThe high off-scale MIC value, >163.5 μM, was converted to the next highest concentration, 327 μM, for calculation of FIC Index. The low off-scale MIC values were converted to the next lowest concentration (one-fold serial dilution) for calculation of the FIC Index.

To test whether the copolymer could act synergistically with fluconazole, which is commonly used to treat candidiasis, we exposed two Candida strains to MM:TM and fluconazole at the same time. Checkerboard tests were used to determine the fractional inhibitory concentrations (FIC) of the combination of fluconazole and MM:TM copolymer against the FLC-sensitive strain SC5314 and FLC-resistant strain K1. Assays were carried out at 30° C. and ambient levels of CO₂ to maintain the yeast morphology of the strains. Fluconazole and MM:TM in combination were synergistic (_{Σ} FIC Index value of 0.07) against the fluconazole resistant K1 strain (Table 2). Fluconazole MICs against the K1 strain in combination with copolymer decreased dramatically, resulting in a ≥100-fold improvement in antifungal activity.

### MM:TM Copolymer Shows Activity Against Multiple Species of Cryptococcus:

We also determined the MICs of MM:TM against four strains of C. neoformans and two strains of C. gattii using the CLSI M27-A3 broth microdilution method (Table 5). MIC assays were tested against the C. neoformans H99 strain at cell concentrations of 1.25×10³ and 1.25×10⁵ cells/mL. As was seen with Candida spp., both concentrations provided consistent results in all cases (data not shown). The MICs of the MM:TM copolymer against all Cryptococcus spp. tested, were low at <1 μm in all cases (Table 3). In all cases the MICs were equal or within one dilution of the MFCs, indicating that MM:TM is fungicidal against these Cryptococcus species, including a rapamycin resistant strain (C₂₁F3) (Tables 14-17, below). For comparison of MM:TM activity to currently used antifungal drugs, MIC and MFC values for the Cryptococcus test strains were determined using AMB and FLC, and all were in the expected range based on previous reports: ˜1 μM (MIC and MFC) for AMB and ≤10 μm (MIC) and >100 μM (MFC) for FLC against all strains with the exception of C₂₁F3, which was killed by fluconazole at ˜5 μM (Tables 3, 14, and 15).

TABLE 3
MIC and synergy data for MM:TM against Cryptococcus spp.
Cryptococcus spp. MIC Results by Broth Microdilution (μM)^{a,b}
Inoculum concentration of 1.25 × 10³ cells/mL at 30° C.

| | MM:TM^{c} | AMB^{d} | FLC^{e} |
|---|---|---|---|
| Isolate | MIC (μM) | MIC (μM) | MIC (μM) |
| C. neoformans | 0.5 | 0.2 | 5.2 |
| (JEC21) | | | |
| C. neoformans | 0.9 | 0.9 | 5.2 |
| (B3501) | | | |
| C. neoformans | 0.9 | 1.7 | 10.1 |
| (H99) | | | |
| C. neoformans | 0.9 | 0.9 | 5.2 |
| (C21F3) | | | |
| C. gattii | 0.9 | 0.9 | 2.6 |
| (WM276) | | | |
| C. gattii | 0.9 | ≤0.2 | 5.2 |
| (C751) | | | |

Cryptococcus neoformans synergy checkerboard results with
AMB and MM:TM (μM)^{a,b} Inoculum concentration of
1.25 × 10⁵ cells/mL at 30° C.

| | | MIC (μM) | | ΣFIC | FIC |
|---|---|---|---|---|---|
| Isolate | Test agent | Alone^{f} | Combined^{g} | Index^{h} | Interpret. |
| H99 | MM:TM | 2.9 | 0.01 | 0.08 | Synergistic |
| | AMB | 3.4 | 0.16 | | |

^{a}MIC, minimum inhibitory concentration
^{b}Each experiment was repeated in duplicate on separate days in at least two different trial experiments
^{c}Average molecular weight of MM:TM copolymer used for molarity conversion is 3300 g/mol
^{d}AMB, amphotericin B (MW = 924.091 g/mol);
^{e}FLC, fluconazole (MW = 306.271 g/mol)
^{f}MIC as determined by OD₆₀₀ measurements after 48 hours for compound as a single agent
^{g}MIC as determined by OD₆₀₀ measurements after 48 hours for compounds incubated with Cryptococcus in combination.
^{h}Fractional inhibitory concentration (FIC)

To test whether the copolymer could act synergistically with AMB, which is commonly used to treat cryptococcosis, we exposed the virulent type strain (H99) to MM:TM and AMB at the same time. Checkerboard tests were used to determine the fractional inhibitory concentrations (FIC) of the combination of AMB and MM:TM copolymer. We determined that AMB and MM:TM acted synergistically against H99 (_{Σ}IC Index value of 0.1), decreasing the MIC of AMB by >10-fold, and this synergy was fungicidal (Table 3).

MM:TM Copolymer Shows Synergism with Azole Drugs Against Aspergillus spp.:

We also determined the MICs of MM:TM against four strains of A. fumigatus and one strain of A. terreus (Table 5). In line with our findings with Candida spp. and Crptococcus spp., we determined that the MM:TM copolymer shows antifungal activity against Aspergillus spp., but not to the level as shown against Candida spp., displaying MIC values >60.6 μM in all cases (Table 4). For comparison of MM:TM activity to currently used antifungal drugs often used to treat aspergillosis, MIC and MFC values for the Aspergillus test strains were determined using posaconazole and itraconazole, and all were in the expected range based on previous reports: ˜1 μM for both azoles against azole sensitive strains and >45 μM for the azole resistant strain F11628. (Perfect J R, Lang S D, Durack D T. 1980. Chronic cryptococcal meningitis: a new experimental model in rabbits. Am J Pathol 101:177-194.) Strain F16216 was sensitive to posaconazole (2.5 μM) and resistant to itraconazole (45.3 μM) (Table 4).

To identify possible synergistic activity against Aspergillus, we tested MM:TM and the azoles in combination. Using a checkerboard test, we determined the FICs of the azole drugs and MM:TM against both azole sensitive and resistant A. fumigatus strains. Overall, we found that MM:TM exhibits synergistic activity with both posaconazole and itraconazole against both sensitive and resistant strains of A. fumigatus (Table 5). Specifically, there was weak synergy (_{Σ}FIC Index values from 0.1-0.1) with both azoles against azole-sensitive strains (AF293 and CEA10), resulting <7-fold increases in efficacy in the presence of copolymer. In contrast, synergy with both azoles against the azole-resistant strain F11628 was very strong (_{Σ}FIC Index values of 0.02 and 0.04), resulting in >600-fold and >100-fold increases in efficacy in the presence of MM:TM for posaconazole and itraconazole, respectively (Table 4).

TABLE 4
MIC and Synergy Data for MM:TM Against Aspergillus spp.
MIC Results by Broth Microdilution (μM)^{a,b}

| | MM:TM^{c} | Posaconazole^{d} | Itraconazole^{e} |
|---|---|---|---|
| Isolate | MIC (μM) | MIC (μM) | MIC (μM) |
| A. terreus | >60.6 | 1.4 | 1.4 |
| NIH2624 | | | |
| A. fumigatus | >60.6 | 2.9 | 1.4 |
| AF293 | | | |
| A. fumigatus | >60.6 | 0.7 | >1.4 |
| CEA10 | | | |
| A. fumigatus | >60.6 | >45.6 | 68.0 |
| F11628 | | | |
| A. fumigatus | >60.6 | 2.5 | 45.3 |
| F16216 | | | |

Synergy Results with azoles and MM:TM (40:60)^{b}

| | | MIC (μM) | | | |
|---|---|---|---|---|---|
| | | | Com- | ΣFIC | FIC |
| Isolate | Test agent | Alone^{f} | bined^{h} | Indexⁱ | Interpret. |
| A. | MM:TM | >30.3ⁱ | 1.2 | 0.23 | Synergistic |
| fumigatus | Posaconazole | 1.6 | 0.3 | | |
| AF293 | MM:TM | >30.3ⁱ | 1.2 | 0.22 | Synergistic |
| | Itraconazole | 1.7 | 0.3 | | |
| A. | MM:TM | >30.3ⁱ | 0.5 | 0.20 | Synergistic |
| fumigatus | Posaconazole | 0.7 | 0.1 | | |
| CEA10 | MM:TM | >30.3ⁱ | 0.5 | 0.11 | Synergistic |
| | Itraconazole | >1.4 | 0.3 | | |
| A. | MM:TM | >30.3ⁱ | 1.2 | 0.02 | Synergistic |
| fumigatus | Posaconazole | 102.7 | 0.1 | | |
| F11628 | MM:TM | >30.3ⁱ | 2.1 | 0.04 | Synergistic |
| | Itraconazole | 102.0 | 0.6 | | |
| A. | MM:TM | >30.3ⁱ | 0.5 | 0.57 | Indifferent |
| fumigatus | Posaconazole | 2.5 | 1.4 | | |
| F16216 | MM:TM | >30.3 | >30.3 | 1.44 | Indifferent |
| | Itraconazole | 90.7 | 85.0 | | |

^{a}MIC, minimum inhibitory concentration
^{b}Each experiment was repeated in duplicate on separate days in at least two different trial experiments
^{c}Average molecular weight of MM:TM copolymer used for molarity conversion is 3300 g/mol
^{d}Posaconazole (MW = 700.778 g/mol);
^{e}Itraconazole (MW = 705.64 g/mol)
^{f}MIC after 48 hours for compound as a single agent
^{g}MIC after 48 hours for compounds incubated with Aspergillus in combination.
^{h}Fractional inhibitory concentration (FIC)
ⁱThe high off-scale MIC value, >30.3 μM, was converted to the next highest concentration, 60.6 μM, for calculation of FIC Index.

### MM:TM Shows Relatively Low Toxicity to Diverse Host Cells:

After observing that the inhibitory and synergistic concentrations of the copolymer were within clinically relevant ranges, we determined the effects of MM:TM on host cells at and above those concentrations. We also compared MM:TM toxicity to currently used antifungals. We tested cells with varied functions, including structural fibroblasts (NIH 3T3), immune cells in the form of macrophages (RAW 264.7), and human red blood cells (hRBCs) to determine whether differences between cell types altered sensitivity to MM:TM. Fibroblasts and macrophages were incubated for 12 hours, and hRBCs for 1 hour, in the presence of a dilution series of copolymer, AMB, or FLC (hRBCs were not tested with fluconazole). The percentage of cells lysed by the treatments was measured by release of lactate dehydrogenase (LDH) (fibroblasts and macrophages) or hemoglobin (hRBCs) and compared to 0% lysis (no treatment) and 100% lysis (triton) controls to determine the amount of each drug that would cause 10% and 50% cell death.

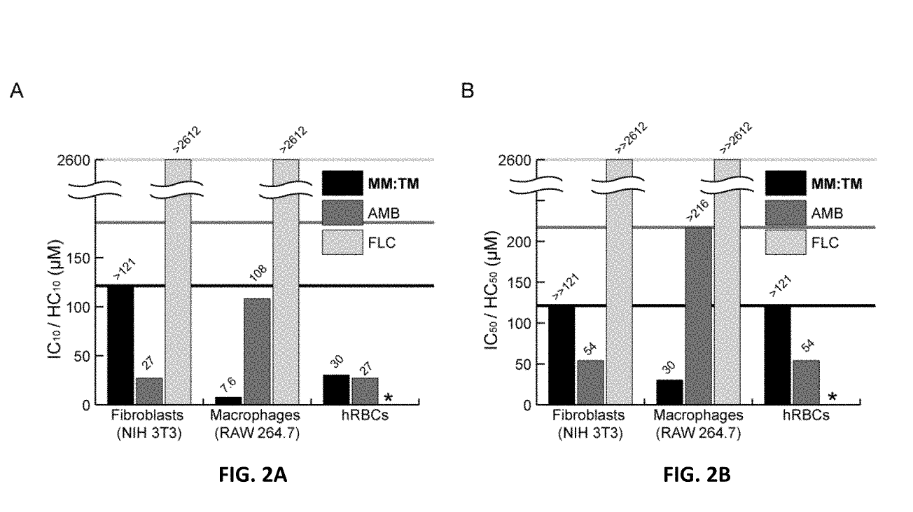

We observed that MM:TM exhibited very low toxicity to fibroblasts. At the highest concentration tested (121 μM), which was ˜10× and 100× higher than the MIC against Candida and Cryptococcus, respectively, fewer than 10% of fibroblasts were killed. MM:TM was also significantly less toxic than AMB to fibroblasts by a factor of at least 4. In contrast, macrophages showed increased susceptibility to copolymer with 10% and 50% cell death observed at 7.6 and 30 μM respectively. AMB was less toxic to macrophages compared to MM:TM, causing 10% cell death at 108 μM, and less than 50% cell death at the highest concentration tested (FIG. 2). While hRBCs showed 10% hemolysis at similar concentrations of MM:TM and AMB (30 and 27 μM respectively), lower concentrations of amphotericin B caused 50% lysis of hRBCs (54 μM); whereas 50% cell death was never achieved, even at the highest concentrations of MM:TM tested. From these data, we conclude that MM:TM shows relatively low toxicity against fibroblasts and hRBCs relative to AMB and ˜10-fold higher toxicity toward macrophages than AMB. These findings show that MM:TM has utility to treat fungal infections. That is, the compounds are fungicidal at concentrations not unduly damaging to human cells. The increased activity of MM:TM against macrophages suggests that there may be different mechanisms and cellular responses, based on intrinsic cell function.

Finally, we tested MM:TM against Arabidopsis seedlings to assess its effects on plant growth. Antifungals, including azoles, have been used in agriculture to curb crop loss due to plant fungal pathogens. Root growth (in cm) was determined 6 and 10 days after germination of seeds in 0, 0.3, 3.0, and 30 μM MM:TM. At 0.3 μM and 3.0 μM MM:TM seedlings appeared to grow normally, and root growth was not statistically different from the controls. At 30 μM MM:TM, however, root growth decreased by roughly 40%, and the roots themselves displayed an unusual waving and rightward skewing behavior (data not shown). These results indicate that at high concentrations, the MM:TM copolymer has negative effects on A. thaliana, but at lower concentrations (to which fungi are susceptible), MM:TM does not adversely affect germination or growth of A. thaliana.

Despite the great promise of HDPs in their native forms as antimicrobial agents, the cost of producing sequence-specific oligomers in a consistent, large-scale manner serves as a significant barrier to their development for therapeutic use. These limitations have led many research groups to study the antimicrobial properties of synthetic, amphipathic polymers. The study of antimicrobial polymers has been inspired by the observation that despite the diverse amino acid composition and size of most HDPs, they maintain similar growth inhibitory activities against bacteria. Therefore, it has been hypothesized that cationic and hydrophobic side chain-containing copolymers form irregular conformations that result in global amphiphilicity when in contact with bacterial membranes, thereby eliciting antimicrobial activity. (Mowery B P, Lindner A H, Weisblum B, Stahl S S, Gellman S H. 2009. Structure—activity Relationships among Random Nylon-3 Copolymers That Mimic Antibacterial Host-Defense Peptides. J Am Chem Soc 131:9735-9745. Tossi A, Sandri L, Giangaspero A. 2000. Amphipathic, α-helical antimicrobial peptides. Pept Sci 55:4-30.) Nylon-3 copolymers were among the first reported HDP-mimetic synthetic polymers displaying broad spectrum growth inhibition against a diverse panel of bacterial species with low hemolytic activity. (Mowery B P, Lee S E, Kissounko D A, Epand R F, Epand R M, Weisblum B, Stahl S S, Gellman S H. 2007. Mimicry of Antimicrobial Host-Defense Peptides by Random Copolymers. J Am Chem Soc 129:15474-15476.) The activities of nylon-3 polymers against fungi have been less promising because antifungal activity has historically correlated directly with high hemolytic activity, suggesting poor selectivity for fungal cells relative to mammalian cells. (Karlsson A J, Pomerantz W C, Weisblum B, Gellman S H, Palecek S P. 2006. Antifungal Activity from 14-Helical β-Peptides. J Am Chem Soc 128:12630-12631.) Here we report the development and characterization of a new cationic HDP-mimicking copolymer, MM:TM, that shows excellent activity against a diverse set of invasive human fungal pathogens but little toxicity toward mammalian cells. Nylon-3 polymers are synthesized by the AROP of β-lactams to generate sequence random polymers with narrow molecular weight distributions. The backbones of nylon-3 polymers are analogous to those of natural α-peptides because they contain secondary amide bonds; however, they have one significant difference—the space between amide bonds is extended by one extra carbon atom. This difference is valuable because it prevents proteolysis of nylon-3 polymers by all natural proteases, leading to exceptional polymer stability.

Three model systems were used to test the antifungal activity of the subject compounds: Candida, Aspergillus, and Cryptococcus. These fungal genera were chosen for three reasons: 1) clinical relevance; 2) phylogenetic diversity and breadth; and 3) wide use as model systems, both in vitro and in vivo. All three cause deadly invasive fungal diseases and account for >75% of fatal fungal disease in humans. Two belong to the phylum of ascomycetes (Candida and Aspergillus), each with diverse properties (yeast vs. filamentous fungus), and one is a divergent, basidiomycete yeast (Cryptococcus). Within these genera, we were able to increase the diversity by testing a number of species, all of which are clinically relevant. Finally, these three fungi have been the subject of intense study and represent model systems in the field of mycology. As such, there is a wealth of information from these systems as well as genetic, molecular, bioinformatic, and animal tools available for each. All of these resources will be invaluable for the future determinations of mechanisms of MM:TM action.

Through our studies, we found that the nylon-3 copolymer, MM:TM, displays broad-spectrum antifungal activity against Candida, Cryptococcus, and Aspergillus with minimal hemolytic activity. The MM:TM copolymer displays good activity against C. albicans and excellent activity against C. neoformans as a stand-alone agent. The MM:TM polymer displays strong synergistic activity with azole drugs against C. albicans and A. fumigatus, even against azole-resistant strains. The remarkable activity of this copolymer, a non-traditional chemotype for drug development, against diverse fungi suggests that optimization could lead to development of an effective broad-spectrum antifungal agent. The chemistry of nylon-3 polymers allows for easy optimization of polymer structure and composition for development of either broad-spectrum or customized polymers with activity against a particular fungus.

Current antifungals (particularly amphotericin B) suffer with problems of toxicity to the human host (Odds F C, Brown A J P, Gow N A R. 2003. Antifungal agents: mechanisms of action. Trends Microbiol 11:272-279), and this is a key issue to be resolved in the development of new drugs. We discovered that the toxicity of MM:TM varies against mammalian cells with different functions, with structural cells (fibroblasts) and blood cells proving resistant, whereas macrophages were relatively sensitive to the copolymer. While creating challenges for some applications, macrophage toxicity could prove to be a beneficial tool against fungi (including Cryptococcus) that have been proposed to use phagocytes as Trojan horses to disseminate in the host or to lay dormant for long periods of time. (Mansour M K, Vyas J M, Levitz S M. 2011. Dynamic virulence: real-time assessment of intracellular pathogenesis links Cryptococcus neoformans phenotype with clinical outcome. mBio 2. Feldmesser M, Kress Y, Novikoff P, Casadevall A. 2000. Cryptococcus neoformans is a facultative intracellular pathogen in murine pulmonary infection. Infect Immun 68:4225-4237.) Targeting fungi within host macrophages could be a killing strategy and/or lead to insights into the mechanism of MM:TM action. Future efforts to determine mammalian cells that exhibit killing by or resistance to the copolymer promise to be extremely revealing. We also investigated toxicity of the copolymer to plant cells using the model plant, A thaliana. Root inhibition and morphological abnormalities in A. thaliana were observed at high concentrations but not lower concentrations, indicating that the copolymer could have applications in agriculture at lower (but still fungicidal) concentrations.

The MM:TM copolymer was designed to mimic the antimicrobial function of natural host-defense peptides, containing a substantial number of cationic and hydrophobic subunits. The antimicrobial properties of HDPs and their polymer mimics are hypothesized to arise in part due to their ability to disrupt bacterial membranes. (Zasloff M. 2002. Antimicrobial peptides of multicellular organisms. Nature 415:389-395. Mookherjee N, Hancock R E, W. 2007. Cationic host defense peptides: Innate immune regulatory peptides as a novel approach for treating infections. Cell Mol Life Sci 64:922-33.) The model is that the positive charge of HDPs and antimicrobial polymers interact with the negatively charged membranes of bacteria selectively compared to that of eukaryotic cells, whose membranes have a greatly reduced negative charge. The hydrophobic subunits of HDPs and their mimics are required for insertion of these molecules into membranes to enable membrane disruption. ¹H NMR analysis of the MM:TM copolymer reveals that these copolymers are highly cationic in nature (roughly 80% cationic subunit to 20% hydrophobic subunit), containing roughly three hydrophobic subunits per copolymer with an average chain length of 15. This overrepresented proportion of cationic subunits coupled with previously published antifungal activity of the nylon-3 homopolymer, NM₂₀, containing only cationic subunits, suggests that these polymers act via a different mechanism than traditional HDPs.

### Pharmaceutical Compositions:

Pharmaceutical compositions comprising the nylon-3 copolymers disclosed herein may be prepared by any of the methods well known in the art of pharmacy. All methods include the step of bringing the active ingredient into association with a carrier which constitutes one or more accessory ingredients. In general, the compositions are prepared by uniformly and intimately bringing the active ingredient into association with a liquid or solid carrier and then, if necessary, shaping the product into the desired unit dosage form. For inhaled compositions, the unit dosage may be pre-packaged into an inhaler, nebulizer, or the like, or pre-packaged into single-use containers than can be opened and dispensed using an inhaler or nebulizer. The compositions may also be delivered to the lungs intratracheally (via a breathing tube) as a liquid bolus. The compositions may contain conventional adjuvants such as buffers, bacteriostats, viscosity-altering agents, and the like. The compositions may be presented in unit dose or multi-dose containers, for example, sealed vials.

Compositions suitable for inhalation or instillation administration may include a micronized powder formulation (if the carrier is a solid) having a particle size in the range of from about 5 microns or less to about 500 microns, or a liquid formulation, for rapid inhalation through the nasal or oral passage from a conventional inhaler, nebulizer, or the like. Suitable liquid nasal compositions include conventional nasal sprays, nasal drops and the like, of aqueous solutions of the active ingredient and optional adjuvants.

In addition to the aforementioned ingredients, the compositions of this invention may further include one or more optional accessory ingredients(s) utilized in the art of pharmaceutical formulations, e.g., diluents, buffers, flavoring agents, colorants, binders, surfactants, thickeners, lubricants, suspending agents, preservatives (including antioxidants) and the like.

The compositions may be used to treat, inhibit, or otherwise ameliorate fungal infections and re-infections in mammals, including human beings. More specifically, the pharmaceutical composition may comprise one or more of the nylon-3 polymers as well as a standard, well-known, non-toxic pharmaceutically suitable carrier, adjuvant or vehicle such as, for example, phosphate buffered saline, water, ethanol, polyols, vegetable oils, a wetting agent, or an emulsion such as a water/oil emulsion. The composition may be in either a liquid, solid, or semi-solid form. For example, the composition may be in the form of a tablet, capsule, ingestible liquid or powder, injectible, suppository, or topical ointment or cream. Proper fluidity can be maintained, for example, by maintaining appropriate particle size in the case of dispersions and by the use of surfactants. It may also be desirable to include isotonic agents, for example, sugars, sodium chloride, and the like. Besides such inert diluents, the composition may also include adjuvants, such as wetting agents, emulsifying and suspending agents, sweetening agents, flavoring agents, perfuming agents, and the like.

Suspensions, in addition to the active compound(s), may comprise suspending agents such as, for example, ethoxylated isostearyl alcohols, polyoxyethylene sorbitol and sorbitan esters, microcrystalline cellulose, aluminum metahydroxide, bentonite, agar-agar and tragacanth or mixtures of these substances.

Solid dosage forms such as tablets and capsules can be prepared using techniques well known in the art of pharmacy. For example, nylon-3 polymers produced as described herein can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant nylon-3 polymer(s).

For intravenous administration, the nylon-3 polymers may be incorporated into commercial formulations such as Intralipid©-brand fat emulsions for intravenous injection. (“Intralipid” is a registered trademark of Fresenius Kabi A B, Uppsalla, Sweden.) Where desired, the individual components of the formulations may be provided individually, in kit form, for single or multiple use. A typical intravenous dosage of a representative nylon-3 polymer as described herein is from about 0.1 mg to 100 mg daily and is preferably from 0.5 mg to 3.0 mg daily. Dosages above and below these stated ranges are specifically within the scope of the claims.

Possible routes of administration of the pharmaceutical compositions include, for example, enteral (e.g., oral and rectal) and parenteral. For example, a liquid preparation may be administered, for example, orally or rectally. Additionally, a homogenous mixture can be completely dispersed in water, admixed under sterile conditions with physiologically acceptable diluents, preservatives, buffers or propellants in order to form a spray or inhalant. The route of administration will, of course, depend upon the desired effect and the medical stated of the subject being treated. The dosage of the composition to be administered to the patient or subject may be determined by one of ordinary skill in the art and depends upon various factors such as weight of the patient, age of the patient, immune status of the patient, etc., and is ultimately at the discretion of the medical professional administering the treatment. Veterinary and agricultural use is also within the scope of the method, and thus the species of the subject being treated would also be taken into account.

With respect to form, the composition may be, for example, a solution, a dispersion, a suspension, an emulsion, or a sterile powder which is then reconstituted. The composition may be administered in a single daily dose or multiple doses.

The present disclosure also includes treating, inhibiting, and/or otherwise amelioriating fungal infections and re-infections in mammals, including humans, by administering a fungal grown inhibitory-effective amount of one or more of the nylon-3 polymers disclosed herein. In particular, the compositions may be used to treat fungal infections of any and all description, at any growth stage of the organism.

It should be noted that the above-described pharmaceutical compositions may be utilized in connection with non-human animals, both domestic and non-domestic, as well as humans.

Additionally, the nylon-3 polymers described herein may also be used in conjunction with other, distinct antifungal agents in a combination antifungal therapy. Thus, the nylon-3 polymers described herein may be admixed with well-known antifungal agents such as polyene antifungal agents (e.g., amphotericin B, candicidin, filipin, hamycin, natamycin, nystatin, rimocidin, and the like), imidazole, triazole, and thiazole antifungal agents (e.g., bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, and the like), allylamines (e.g., amorolfin, butenafine, naftifine, terbinafine, and the like), and echinocandins (e.g., anidulafungin, caspofungin, micafungin, and the like). The combination therapy yields unexpectedly synergistic results in inhibiting the growth of fungi.

### EXAMPLES

Reagents:

Protected α-amino acids and resins used in peptide synthesis were purchased from Novabiochem (a wholly owned subsidiary of EMD Chemicals Inc. and Merck KGaA, Darmstadt, Germany). Protected β³-amino acids were purchased from PepTech (Burlington, Mass., USA). Cyclically constrained β-residues, Fmoc-ACPC and Fmoc-APC(Boc), were prepared as previously described. Lee, LePlae, Porter, and Gellman (2001) J. Org. Chem. 66:3597-3599; LePlae, Umezawa, Lee, and Gellman (2001) J. Org. Chem. 66:5629-5632. 2-(1H-Benzotriazole-1-yl)-1,1,3,3-tetramethylaminium hexafluoro-phosphate (HBTU) was purchased from AnaSpec (San Jose, Calif., USA). 5-Carboxyfluorescein was purchased from Invitrogen (Carlsbad, Calif., USA). 1-Methyl-2-pyrollidinone (NMP) was purchased from Advanced Chemtech (Louisville, Ky., USA). All other reagents were purchased from Sigma-Aldrich Corp. (St. Louis, Mo., USA) or Fisher Scientific (Pittsburgh, Pa., USA) and used as received.

Synthesis:

All peptides were prepared on “NovaSyn TGR”-brand resin (Novabiochem). α-Peptides were prepared by standard Fmoc solid phase peptide synthesis methods on a Symphony Multiple Peptide Synthesizer (Protein Technologies, Inc., Tucson, Ariz., USA). α/β-Peptides were prepared by automated Fmoc solid phase peptide synthesis on a Synergy 432A automated synthesizer (Applied Biosystems, Foster City, Calif., USA). α/β-Peptides were also prepared manually by microwave-assisted Fmoc solid phase peptide synthesis. Erdelyi and Gogoll (2002) Synthesis 11:1592-1596. The N-terminus of each peptide was capped by treatment with 8:2:1 DMF/DIEA/Ac₂O. The resin was washed thoroughly (3×DMF, 3×CH₂Cl₂, 3×MeOH) and then dried under vacuum. All peptides were cleaved from resin by treatment with 94:2.5:2.5:1 TFA/H₂O/ethanedithiol/triisopropylsilane. The resin was filtered, washed with additional TFA, and the combined filtrates concentrated to ˜2 mL under a stream of dry nitrogen. Crude peptide was precipitated from the cleavage mixture by addition of cold ether (45 mL). The mixture was centrifuged, decanted, and the remaining solid dried under a stream of nitrogen. Peptides were purified by reverse phase HPLC on a prep-C₁₈ column using gradients between 0.1% TFA in water and 0.1% TFA in acetonitrile. The identity and purity of the final products were confirmed by MALDI-TOF-MS and analytical HPLC, respectively. Stock solution concentrations were determined by UV absorbance. Gill, S. C.; Vonhippel, P. H. (1989) Anal. Biochem. 182:319-326.

Fungal Strain Maintenance:

All strains were handled using standard techniques and media as described previously. (Sherman F, Hicks J. 1987. Methods in Yeast Genetics: A Laboratory Course Manual. Cold Spring Harbor Laboratory.) Candida and Cryptoccocus strains were grown on yeast extract peptone dextrose (YPD) agar plates and stored at 4° C. Candida and Cryptococcus were cultured overnight in liquid media at 30° C. and washed with PBS (phosphate buffered saline) prior to minimum inhibitory concentration (MIC), minimum fungicidal concentration (MFC), and synergy studies. Aspergillus strains were maintained as glycerol stocks at ˜80° C. and propagated on glucose minimal medium (GMM) at 37° C. Spores were harvested in 0.01% Tween 20, enumerated using a hemacytometer, and used for MIC, MFC, and synergy studies immediately post-harvest.

TABLE 5
Strains used in this study

| | | | | | |
|---|---|---|---|---|---|
| | | | Drug | Features and | |
| Species | Strain name | Origin | Resistances | markers | Source |

Candida

| | | | | | |
|---|---|---|---|---|---|
| C. albicans | SC5314 | Clinical | — | | ATCC MYA-2876 |
| C. lusitaniae | CL3 | Clinical | — | MATalpha | ATCC 42720 |
| C. albicans | K1 | Clinical | Fluconazole^{R} | | Gifted from Bruce |
| | | | | | Klein Lab |
| C. albicans | Gu5 | Clinical (Germany) | Fluconazole^{R} | | ATCC MYA-574 |
| C. albicans | E4 | Clinical | Polyene | | ATCC 38248 |
| | | | antibiotics^{R} | | |
| | | | (Amphotericin | | |
| | | | B^{R}) | | |

Cryptococcus

| | | | | | |
|---|---|---|---|---|---|
| C. neoformans | H99 | Clinical (NY, US) | — | SeroA, | ATCC 208821 |
| | | | | MATalpha | |
| C. neoformans | B3501 | Single progeny from | — | SeroD, | ATCC 3487 |
| | | cross ATCC 28957 × | | MATalpha | |
| | | ATCC 28958 | | | |
| C. neoformans | JEC21 | Derived from B- | — | SeroD, | ATCC 96910 |
| | | 3501 and B-3502 | | MATalpha | |
| C. neoformans | C21F3 | Derived from JEC 21 | rapamycin^{R} | SeroD | ATCC MYA-737 |
| | | (ATCC 96910) | FK506^{R} | Does not express | |
| | | | | FKBP12 | |
| C. gattii | WM276 | Environmental | — | SeroB, MATalpha | ATCC 4071 |
| | | (Sydney, AU) | | | |
| C. gatti | C751/PNG9 | Clinical (Papua New | — | SeroB, MATalpha | Hull Laboratory |
| | | Guinea) | | | |

Aspergillus

| | | | | | |
|---|---|---|---|---|---|
| A. fumigatus | AF293 | Clinical (UK) | — | | ATCC MYA-4609 |
| A. fumigatus | CEA10 | Clinical | — | | Keller Laboratory |
| A. fumigatus | F11628 | Clinical (Liverpool, | Azole^{R} | Cyp51A mut. | Keller Laboratory |
| | | UK) | | G138C | |
| A. fumigatus | F16216 | Clinical | Azole^{R} | Cyp51A mut. | Keller Laboratory |
| | | (Northampton, UK) | | L98H + TR | |
| A. fumigatus | 12-7505446 | | Azole^{R} | Cyp51A mut. | Keller Laboratory |
| | | | | L98H + TR | |
| A. fumigatus | 12-7505220 | | Azole^{R} | Cyp51A mut. | Keller Laboratory |
| | | | | L98H + TR | |
| A. fumigatus | 08-12-12-13 | | Azole^{R} | Cyp51A mut. | Keller Laboratory |
| | | | | L98H + TR | |
| A. fumigatus | 08-36-03-25 | | Azole^{R} | Cyp51A mut. | Keller Laboratory |
| | | | | L98H + TR | |
| A. fumigatus | 08-31-08-91 | | Azole^{R} | Cyp51A mut. | Keller Laboratory |
| | | | | L98H + TR | |
| A. fumigatus | 08-19-02-61 | | Azole^{R} | Cyp51A mut. | Keller Laboratory |
| | | | | L98H + TR | |
| A. fumigatus | D5 | | Azole^{R} | Cyp51A mut. | Keller Laboratory |
| | | | | G54E | |
| A. fumigatus | D6 | | Azole^{R} | Cyp51A mut. | Keller Laboratory |
| | | | | G54V | |
| A. fumigatus | D9 | | Azole^{R} | Cyp51A mut. | Keller Laboratory |
| | | | | M220K | |
| A. fumigatus | D7 | | | | Keller Laboratory |
| A. terreus | NIH2624 | | — | | Keller Laboratory |

### Synthesis and Purification of the MM:TM Random Copolymers:

Nylon-3 copolymers were synthesized and purified by literature methods. (Mowery B P, Lee S E, Kissounko D A, Epand R F, Epand R M, Weisblum B, Stahl S S, Gellman S H. 2007. Mimicry of Antimicrobial Host-Defense Peptides by Random

Copolymers. J Am Chem Soc 129:15474-15476.) All polymerizations were performed in a moisture-controlled, N₂-purged glove box at room temperature. Monomethyl-containing (MM) (0.120 mmol) and tetramethyl-containing (TM) (0.180 mmol) β-lactams in dry tetrahydrofuran (THF) (2 mL) were mixed with a 0.05 M solution of the co-initiator, 4-tert-butylbenzoyl chloride (0.015 mmol) in THF to approximate an average 20-mer copolymer chain length and generate an ˜40:60 MM:TM ratio. A 0.05M solution of lithium bis(trimethylsilyl)amide in THF (0.038 mmol) was added, and the reaction was stirred overnight at room temperature. Polymerization reactions were quenched with the addition MeOH (˜200 μL), and the resulting copolymer was isolated through repeated precipitation in pentane. The resulting white pellet was dried under vacuum to constant weight and then characterized by gel permeation chromatography (GPC) using N,N-dimethylacetamide (DMAc) as the mobile phase.

N-tert-butoxycarbonyl protecting groups were removed by treating the side-chain-protected copolymer with neat trifluoroacetic acid (3 mL) at room temperature for 2 hours with shaking. Side-chain-deprotected copolymers were precipitated as a white solid upon the addition of cold diethyl ether. The TFA salt form of the deprotected copolymer was collected after centrifugation, dried under vacuum, and washed with ether. The solid copolymer was then dried under vacuum, dissolved in 5 mL of water, and lyophilized to yield a white, fluffy solid.

### Copolymer Characterization by GPC Using DMAc as the Mobile Phase:

The side-chain amine-protected copolymer was dissolved in DMAc (10 mM LiBr) at a concentration of 2.5 mg/mL and filtered through a 0.45 μm modified hydrophilic polytetrafluoroethylene membrane filter. The gel permeation chromatography (GPC) analysis used two Waters Styragel HR 4E columns (particle size 5 μm) linked in series on a Waters GPC instrument equipped with a refractive index detector (Waters 2410). DMAc containing 10 mM LiBr was used as the mobile phase at a flow rate of 1 mL/min at 80° C. Number-average molecular weight (Mn) and polydispersity index (PDI) were calculated using the Empower software and calibration curves obtained from nine poly(methyl methacrylate) standards with peak average molecular weights (Mp) ranging from 690 to 1944000. The degree of polymerization, as calculated via GPC (Dp_{GPC}), for a particular copolymer was calculated based on the deduced Mn value, the initial ratio of β-lactam monomers used for the reaction, and the molecular weight of the β-lactam monomers. The degree of polymerization, calculated via nuclear magnetic resonance (DP_{NMR}), and subunit ratio for a particular copolymer were calculated based on end group analysis of the tert-butyl benzoyl aromatic protons and the deprotected copolymer lactam proton integration. The copolymer, MM:TM, was found to have an average Mn of 3300 g/mol with an average chain length of 15 monomer subunits.

### Copolymer Characterization by NMR:

Deprotected nylon-3 copolymers were weighed as lyophilized solid and then dissolved in deuterated water at a sample concentration of 8 mg/mL. ¹H NMR spectra were collected on either a Bruker Avance III spectrometer at 400 MHz or 500 MHz at ambient temperature unless otherwise noted. All polymer spectra are reported in ppm using solvent as the internal standard (D₂O at 4.790 ppm). The degree of polymerization (Dp_{NMR}), or average polymer chain length, is calculated from NMR integrations of all copolymer resonances. To calculate the Dp_{NMR}, the tert-butyl benzoyl aromatic protons of the N-terminus of each polymer chain are set to an integration of two, normalizing the monomer integrations for one polymer chain. Assigning each proton resonance to the appropriate monomer subunit, one can calculate the average number of monomer subunits in each polymer chain length. With the average number monomer incorporation for each subunit in hand, the subunit ratio of cationic to hydrophobic monomers can be calculated by dividing the number of cationic or hydrophobic monomer subunits by the Dp_{NMR}. (Tables 6 and 7).

### Antifungal Activity Assays:

The MICs of Candida and Cryptococcus spp. were determined by the broth microdilution method according to the CLSI M27-A3 guidelines. In brief, fungal cells at a concentration of 1.25×10³ cells/mL were incubated with two-fold serial dilutions of MM:TM copolymer (0.5-60.6 μM), Amphotericin B (1.7-21.6 μM), or fluconazole (5.1-653 μM) in RPMI-1640 (0.2% glucose, 0.145 M 3-(N-morpholino)propanesulfonic acid (MOPs), pH 7.0). Assays were carried out in 96 well plates, with 200 μl total volume per well, and incubated for 48 h at 30° C. Wells containing fungal cells with no drug and wells containing only RPMI-1640 (Life Technologies) were used as positive and blank controls. After 48 hr, the OD₆₀₀ of each well was measured using a microplate reader. Percent cell growth was determined as [(sample absorbance−blank absorbance)/(control absorbance−blank absorbance)]×100%. The MIC endpoint of the antifungal agent was determined as the lowest concentration to inhibit 100% of fungal growth compared to the no drug control. Experiments were repeated in duplicate on different days. All values reported represent the average MIC concentration of at least two trials. The average MIC concentration consistently fell within a two-fold serial dilution of the concentration of each experimental replicate.

The MFC was obtained after performing the MIC assay described above using a higher concentration of 1.25×10⁵ cells/mL. After co-incubation with drug, 3 μL of the cell suspension was plated onto yeast extract peptone dextrose (YPD) agar to assess viability. MFC was determined as the lowest copolymer concentration to result in zero fungal colonies.

The MICs of Aspergillus spp. were determined by the broth microdilution method according to the EUCAST-AST-ASPERGILLUS guidelines. (European Committee on Antimicrobial Susceptibility Testing, M. C. Arendrup et al., “EUCAST antifungal MIC methods for moulds,” E.DEF 9.3, 3 Dec. 2015.) Briefly, Aspergillus conidia at a concentration of 1×10⁵ conidia/mL were incubated with two-fold serial dilutions of MM:TM copolymer (0.5-60.6 μM), posaconzole (2.2-285.4 μM), or itraconazole (2.2-283.4 μM) in RPMI-1640. Wells containing fungal cells with no drug and wells containing only RPMI-1640 were used as positive control and blank wells, respectively. Assays were carried out in 96 well plates, with 200 μL total volume per well, and incubated for 48 h at 35° C. The MIC endpoint of the antifungal agent was determined as the lowest concentration to inhibit 100% of hyphal outgrowth from conidia. Experiments were repeated in duplicate on different days. All values reported represent the average MIC concentration of at least two trials. The average MIC concentration consistently falls within a two-fold serial dilution of the concentration of each experimental replicate.

### Synergy Studies:

For Candida albicans and Cryptococcus neoformans synergistic drug interactions were evaluated using a checkerboard microdilution method. Fungal cells at a concentration of 1.25×10⁵ cells/mL were incubated with two-fold serial dilutions of either fluconazole (0-81.6 μM) or Amphotericin B (0-6.7 μM) and the nylon-3 copolymer MM:TM (0-15.1 μM) in RPMI-1640. Assays were carried out as described above. Wells containing fungal cells with each drug separately and wells with no drug were used as controls. After 48 hr, the OD₆₀₀ of each well was measured using a microplate reader. The MIC endpoint of the drugs alone or in combination was determined as the lowest concentration to inhibit 100% of fungal growth compared to the no drug control.

For Aspergillus fumigatus synergistic drug interactions were evaluated using a checkerboard microdilution method. Aspergillus conidia at a concentration of 1×10⁵ conidia/mL were incubated with two-fold or one-fourth-fold serial dilutions of either posaconazole (0-68.5 μM), or itraconazole (0-63.8 μM), and nylon-3 copolymer MM:TM (0-30.3 μM) in RPMI-1640. Posaconazole or itraconazole plus copolymer concentrations were tailored according to the antifungal agent and Aspergillus strain used in each assay. Assays were carried out as described above. Wells containing fungal cells with each drug separately, and wells with no drug were used as controls. The MICs of the drugs alone and in combination were determined as the lowest drug concentrations preventing hyphal outgrowth from conidia.

The sum of the fractional inhibitory concentrations (_{Σ}FIC) was calculated as follows for assessing synergy: _{Σ}FIC=_{Σ}FIC_{A}+_{Σ}FIC_{B}, where

$$\sum {FIC}_{A} = \frac{MIC of antifungal A in combination}{MIC of antifungal A alone} .$$

When the MIC of the antifungal agent alone or in combination did not fall within the range of concentrations tested, the next serial dilution higher was used as the MIC. The following values were used as cut-offs: synergism ≤0.5, indifference >0.5 and ≤4, and antagonism >4. (Meletiadis J, Pournaras S, Roilides E, Walsh T J. 2010. Defining fractional inhibitory concentration index cutoffs for additive interactions based on self-drug additive combinations, Monte Carlo simulation analysis, and in vitro-in vivo correlation data for antifungal drug combinations against Aspergillus fumigatus. Antimicrob Agents Chemother 54:602-609.) Interpretation of the checkerboard synergy testing was determined using the method of the Lowest FIC Index. (Bonapace C R, Bosso J A, Friedrich L V, White R L. 2002. Comparison of methods of interpretation of checkerboard synergy testing. Diagn Microbiol Infect Dis 44:363-366.)

### Hemolysis:

Hemolysis assays were performed using wasted or expired human red blood cells (hRBCs) obtained from the University of Wisconsin-Madison Hospital as described previously. (Karlsson A J, Pomerantz W C, Weisblum B, Gellman S H, Palecek S P. 2006. Antifungal Activity from 14-Helical β-Peptides. J Am Chem Soc 128:12630-12631. Raguse T L, Porter E A, Weisblum B, Gellman S H. 2002. Structure—Activity Studies of 14-Helical Antimicrobial β-Peptides: Probing the Relationship between Conformational Stability and Antimicrobial Potency. J Am Chem Soc 124:12774-12785.) Two-fold serial dilutions of MM:TM (1.0-121.2 μM) and amphotericin B (3.4-432.8 μM) dissolved in TRIS-buffered saline (TBS; 10 mM TRIS, 150 mM NaCl, pH 7.2) were incubated with 2% v/v hRBC suspension in TBS. hRBCs treated with TBS only and hRBC treated with a 20% Triton X-100 solution in TBS were used as the blank and positive control, respectively. Assays were carried out in 96 well plates, with 200 μL total volume per well, and incubated for 1 h at 37° C. After incubation, 96-well plates were centrifuged and the supernatant from each well was transferred to a new 96-well plate. The OD₄₀₅ of the supernatants was measured. The percent hemolysis for each sample was calculated as [(sample absorbance−blank absorbance)/(control absorbance−blank absorbance)]×100%.

### In Vitro Cytotoxicity to NIH 3T3 and RAW 264.7 Cells:

Cytotoxicity of the copolymer to NIH 3T3 fibroblasts and RAW 264.7 macrophages was evaluated side-by-side with amphotericin B and fluconazole using the CytoTox-ONE assay kit (Promega, Madison, Wis.). Briefly, in 96 well plates, 1.5×10⁴ NIH 3T3 cells/well in DMEM+10% fetal bovine serum (FBS) or 1×10⁴ RAW 264.7 cells/well in RPMI+10% FBS were plated and allowed to adhere for 24 hours at 37° C. and 5% CO₂. After 24 hours, medium was removed, and 2-fold serial dilution series of copolymer (0.9-121 μM), amphotericin B (1.7-216 μM), or fluconazole (20.4-2612 μM) were added to the wells in triplicate with a total volume of 1000 per well. Plates were incubated at 37° C. and 5% CO₂ for 12 hours. Cells in culture medium only served as the negative toxicity control (blank), and cells treated with a lysate solution of Triton-X 100 to cause 100% release (full toxicity) served as a positive control. Fluorescence intensity was measured on a Tecan Infinite M1000 microplate reader using ex/em 560/590 nm. Cytotoxicity was calculated as (% cell death=(F^{treatment}−F^{blank})/(F^{lysed}−F^{blank})×100%). IC₁₀ and IC₅₀ were determined as the concentration of drug that caused 10% or 50% cell death respectively (or the lower of the two dilutions that fall on either side of 10% or 50% cell death).

### Toxicity to Arapidopsis:

To assess any gross effect of copolymer on plant cell viability, 14 Col-0 Arabidopsis thaliana seedlings were plated on the tops of two half-strength Low Salt medium 1.5% agar plates containing 0.3, 3.0 or 30.3 μM of the nylon-3 copolymer (in triplicate). Plates were stratified at 4° C. in darkness for 48 hours to promote seedling germination, and then transferred to a growth chamber (16 hour day lengths, 21-24° C.). Root growth was marked after 6 and 10 days. At 10 days, scans were taken and growth measured using the NeuronJ plugin in ImageJ. (Schneider C A, Rasband W S, Eliceiri K W. 2012. NIH Image to ImageJ: 25 years of image analysis. Nat Methods 9:671-675.) Average root growth and standard error was determined for all germinated seedlings (minimum of 37 per condition) and statistical differences were assessed using a Student's t-test.

Materials and Instrumentation: 3-(N-morpholino)propanesulfonic acid (MOPs, M92020-250.0) was obtained from Research Products International (Mt. Prospect, Ill.). RPMI 1640 (31800-089) was obtained from Life Technologies (Grand Island, N.Y.). Dulbecco's modified eagle medium (DMEM) (11965092) was obtained from Thermo Fisher Scientific (Waltham, Mass.). CytoTox-ONE assay kits (G7892) were obtained from Promega (Madison, Wis.). EasiVial polymethyl methacrylate (PMMA) standards for GPC column calibration (PL2020-0200) were obtained from Polymer Varian (Palo Alto, Calif.). Posaconazole was purchased from Fluka Analytical (St. Louis, Mo.) and itraconazole was purchased from U.S. Pharmacopeial (USP-U.S., Rockville, Md.). All other chemicals were purchased from Sigma-Aldrich and used without further purification. Fungal susceptibility experiments were performed on Falcon (flat-bottom) tissue culture, polystyrene plates (353072) purchased from Corning (Corning, N.Y.). ¹H and ¹³C NMR spectra were collected on either a Bruker Avance III spectrometer at 400 and 100 MHz, respectively, or on a Bruker Avance III spectrometer at 500 and 125 MHz, respectively, at ambient temperature unless otherwise noted. All polymer spectra are reported in ppm using solvent as the internal standard (D₂O at 4.790 ppm).

### Polymer Characterization:

TABLE 6
MM:TM (40:60) polymer batch characterization
Polymer Characterization: MM:TM

| | | | | NMR characterization | | | |
|---|---|---|---|---|---|---|---|
| | | | | | Observed | | |
| | | | | | subunit | | |
| | | | | | ratio | | Av- |
| | | | | | (cationic: | | erage |
| | GPC characterization^{a,b} | | | | hydro- | | Bulk |
| Batch | PDI_{GPC}^{c} | Mn_{GPC}^{d} | Dp_{GPC}^{e} | Dp_{NMR}^{f} | phobic)^{g} | Mn_{NMR} | MW |
| 1^{a} | 1.27 | 4461 | 27 | 15 | 80:20 | 3280 | 3300 |
| 2^{a} | 1.18 | 4529 | 27 | 14 | 79:21 | 3058 | g/mol |
| 3^{a} | 1.20 | 3723 | 22 | 14 | 79:21 | 3723 | |
| 4^{a} | 1.19 | 4715 | 27 | 16 | 81:19 | 3504 | |
| 5^{b} | 1.24 | 2195 | 13^{a} | 12 | 75:25 | 2596 | |
| 6^{a} | 1.16 | 4262 | 21 | 15 | 80:20 | 3280 | |
| 7^{a} | 1.21 | 4168 | 25 | 15 | 80:20 | 3280 | |
| 8^{a} | 1.21 | 3291 | 19 | 13 | 77:33 | 3824 | |

^{a}Side-chain protected polymer characterization by gel permeation chromatography (GPC) using N,N-dimethylacetamide (DMAc) as the mobile phase.
^{b}Side-chain protected polymer characterization by GPC using tetrahydrofuran (THF) as the mobile phase.
^{c}Polydispersity Index
^{d}The number average molecular weight of side-chain protected polymers
^{e}The degree of polymerization, or average polymer chain length, as calculated from Mn_{GPC}.
^{f}The degree of polymerization, or average polymer chain length, as calculated by Nuclear Magnetic Resonance (NMR) integrations based on end group analysis of the tert-butyl benzoyl aromatic protons, setting the tert-butyl benzoyl aromatic protons to an integration of one polymer chain.
^{g}The subunit ratio of cationic to hydrophobic monomer incorporation for a particular polymer was calculated from NMR integrations, setting the tert-butyl benzoyl aromatic protons to an integration representative of one polymer chain.

TABLE 7
DM:TM (40:60) polymer batch characterization
Polymer Characterization: DM:TM

| | | | | NMR characterization^{c} | | | |
|---|---|---|---|---|---|---|---|
| | | | | | Observed | | |
| | | | | | subunit | | |
| | | | | | ratio | | Av- |
| | | | | | (cationic: | | erage |
| | GPC characterization^{a,b} | | | | hydro- | | Bulk |
| Batch | PDI_{GPC}^{c} | Mn_{GPC}^{d} | Dp_{GPC}^{e} | Dp_{NMR}^{f} | phobic)^{g} | Mn_{NMR} | MW |
| 9^{a} | 1.15 | 4457 | 26 | 14 | 71:29 | 3042 | 3800 |
| 10^{a} | 1.20 | 5038 | 27 | 21 | 67:33 | 4442 | g/mol^{h} |
| 11^{a} | 1.15 | 4703 | 27 | 19 | 63:37 | 3957 | |
| 12^{b} | 1.15 | 1504 | 8 | 8 | 75:25 | 1868 | |

^{a}Side-chain protected polymer characterization by gel permeation chromatography (GPC) using N,N-dimethylacetamide (DMAc) as the mobile phase.
^{b}Side-chain protected polymer characterization by GPC using tetrahydrofuran (THF) as the mobile phase.
^{c}Polydispersity Index
^{d}The number average molecular weight of side-chain protected polymers
^{e}The degree of polymerization, or average polymer chain length, as calculated from Mn_{GPC}.
^{f}The degree of polymerization, or average polymer chain length, as calculated by Nuclear Magnetic Resonance (NMR) integrations based on end group analysis, setting the tert-butyl benzoyl aromatic protons to an integration of one polymer chain.
^{g}The subunit ratio of cationic to hydrophobic monomer incorporation for a particular polymer was calculated from NMR integrations, setting the tert-butyl benzoyl aromatic protons to an integration representative of one polymer chain.
^{h}The molecular weight of batch 13 was not included in the calculation for the average bulk MW of the DM:TM copolymer as this particular batch of copolymer had an abnormally short chain length compared to other batch synthesis.

Candida ssp. Antifungal Studies:

TABLE 8
Candida spp. MIC Results by Broth Microdilution (μg/mL) with an
inoculum concentration of 1.25 × 10³ cells/mL at 35° C.

| | | | Polymer Concentration (μg/mL) | | | |
|---|---|---|---|---|---|---|
| | | | MM:TM | DM:TM | | |
| | | Polymer | (40:60) | (40:60) | AMB^{c} | FLU^{d} |
| Isolate | Trial^{a} | batches^{b} | MIC^{e} | MIC^{e} | MIC^{e} | MIC^{e} |
| C. albicans | 1 | 7.9 | 6.3 | 25 | 3.1 | >200 |
| (SC5314) | 2 | 4.11 | 25 | 6.3 | 6.3 | >200 |
| | avg. | | 15.7 | 15.7 | 4.7 | >200 |
| C. lusitaniae | 1 | 7.9 | 3.1 | 3.1 | <1.6 | >200 |
| (CL3) | 2 | 4.11 | 6.3 | 6.3 | <1.6 | >200 |
| | avg. | | 4.7 | 4.7 | <1.6 | >200 |
| C. albicans | 1 | 7.9 | 25 | 12.5 | <1.6 | >200 |
| (K1) | 2 | 4.11 | 25 | 25 | <1.6 | >200 |
| | avg. | | 25 | 18.8 | <1.6 | >200 |
| C. albicans | 1 | 7.9 | 12.5 | 12.5 | <1.6 | >200 |
| (Gu5) | 2 | 4.12 | 50 | 25 | 3.1 | >200 |
| | avg. | | 31.3 | 18.8 | 2.0 | >200 |
| C. albicans | 1 | 7.9 | 3.1 | 12.5 | 6.3 | >200 |
| (E4) | 2 | 4.11 | 25 | 12.5 | 6.3 | >200 |
| | avg. | | 14.0 | 12.5 | 6.3 | >200 |

^{a}Each experiment was repeated in duplicate on separate days in at least two different trial experiments
^{b}See Tables 6 and 7 polymer batch characterization
^{c}Amphotericin B (AMB)
^{d}Fluconazole (FLU)
^{e}Minimum inhibitory concentration (MIC) as determined by OD₆₀₀ measurements after 24 hours.

TABLE 9
Candida spp. MIC Results by Broth Microdilution (μM) with
an inoculum concentration of 1.25 × 10³ cells/mL at 35° C.

| | | | Polymer Concentration (μM) | | | |
|---|---|---|---|---|---|---|
| | | | MM:TM | DM:TM | | |
| | | Polymer | (40:60)^{c} | (40:60)^{f} | AMB^{e} | FLU^{f} |
| Isolate | Trial^{a} | batches^{b} | MIC^{g} | MIC^{g} | MIC^{g} | MIC^{g} |
| C. albicans | 1 | 7, 9 | 1.9 | 6.6 | 3.4 | >653.0 |
| (SC5314) | 2 | 4, 11 | 7.6 | 1.7 | 6.8 | >653.0 |
| | avg. | | 4.8 | 4.1 | 5.1 | >653.0 |
| C. lusitaniae | 1 | 7, 9 | 0.9 | 0.8 | <1.7 | >653.0 |
| (CL3) | 2 | 4, 11 | 1.9 | 1.7 | <1.7 | >653.0 |
| | avg. | | 1.4 | 1.2 | <1.7 | >653.0 |
| C. albicans | 1 | 7, 9 | 7.6 | 3.3 | <1.7 | >653.0 |
| (K1) | 2 | 4, 11 | 7.6 | 6.6 | <1.7 | >653.0 |
| | avg. | | 7.6 | 4.9 | <1.7 | >653.0 |
| C. albicans | 1 | 7, 9 | 3.8 | 3.3 | <1.7 | >653.0 |
| (Gu5) | 2 | 4, 12 | 15.2 | 6.6 | 3.4 | >653.0 |
| | avg. | | 9.5 | 4.9 | 2.2 | >653.0 |
| C. albicans | 1 | 7, 9 | 0.9 | 3.3 | 6.8 | >653.0 |
| (E4) | 2 | 4, 11 | 7.6 | 3.3 | 6.8 | >653.0 |
| | avg. | | 4.2 | 3.3 | 6.8 | >653.0 |

^{a}Each experiment was repeated in duplicate on separate days in at least two different trial experiments
^{b}See Tables 6 and 7 for polymer batch characterization
^{c}Average molecular weight of MM:TM copolymer used for molarity conversion is 3300 g/mol
^{d}Average molecular weight of DM:TM copolymer used for molarity conversion is 3800 g/mol
^{e}Amphotericin B (AMB), molecular weight = 924.091 g/mol.
^{f}Fluconazole (FLU), molecular weight = 306.271 g/mol.
^{g}Minimum inhibitory concentration (MIC) as determined by OD₆₀₀ measurements after 24 hours.

TABLE 10
Candida spp. MIC Results by Broth Microdilution (μg/mL) with an
inoculum concentration of 1.25 × 10⁵ cells/mL at 30° C.

| | | | Polymer Concentration (μg/mL) | | | | | | | |
|---|---|---|---|---|---|---|---|---|---|---|
| | | | MM:TM | | DM:TM | | | | | |
| | | Polymer | (40:60) | | (40:60) | | AMB^{c} | | FLU^{d} | |
| Isolate | Trial^{a} | batches^{b} | MIC^{e} | MFC^{f} | MIC^{e} | MFC^{f} | MIC^{e} | MFC^{f} | MIC^{e} | MFC^{f} |
| C. albicans | 1 | 1, 9 | 50 | 50 | 50 | 50 | 0.8 | 1.6 | <0.2 | >200 |
| (SC5314) | 2 | 4, 10 | 50 | 100 | 50 | 50 | 0.8 | 1.6 | <0.2 | >200 |
| | avg. | | 50 | 75 | 50 | 50 | 0.8 | 1.6 | <0.2 | >200 |
| C. lusitaniae | 1 | 1, 9 | 12.5 | 25 | 12.5 | 25 | 0.4 | 1.6 | 0.8 | 50 |
| (CL3) | 2 | 4, 10 | 12.5 | 25 | 25 | 37.5 | 0.4 | 1.6 | 0.8 | 100 |
| | avg. | | 12.5 | 25 | 18.8 | 31.3 | 0.4 | 1.6 | 0.8 | 75 |
| C. albicans | 1 | 1, 9 | 50 | 100 | 50 | 100 | 1.6 | 3.1 | >200 | >200 |
| (K1) | 2 | 4, 10 | 100 | 100 | 100 | 100 | 1.6 | 3.1 | >200 | >200 |
| | avg. | | 75 | 100 | 75 | 100 | 1.6 | 3.1 | >200 | >200 |
| C. albicans | 1 | 1, 9 | 50 | 100 | 50 | 50 | 0.8 | 1.6 | >200 | >200 |
| (Gu5) | 2 | 4, 10 | 50 | 100 | 50 | 100 | 0.8 | 1.6 | >200 | >200 |
| | avg. | | 50 | 100 | 50 | 75 | 0.8 | 1.6 | >200 | >200 |
| C. albicans | 1 | 1, 9 | 50 | 100 | 50 | 50 | 3.1 | 12.5 | >200 | >200 |
| (E4) | 2 | 4, 10 | 50 | 100 | 50 | 100 | 3.1 | 12.5 | >200 | >200 |
| | avg. | | 50 | 100 | 50 | 75 | 3.1 | 12.5 | >200 | >200 |

^{a}Each experiment was repeated in duplicate on separate days in at least two different trial experiments
^{b}See Tables 6 and 7 for polymer batch characterization
^{c}Amphotericin B (AMB)
^{d}Fluconazole (FLU)
^{e}Minimum inhibitory concentration (MIC) as determined by OD₆₀₀ measurements after 48 hours.
^{f}Minimum fungicidal concentration (MFC).

TABLE 11
Candida spp. MIC Results by Broth Microdilution (μM) with an inoculum
concentration of 1.25 × 10⁵ cells/mL at 30° C.

| | | | Polymer Concentration (μM) | | | | | | | |
|---|---|---|---|---|---|---|---|---|---|---|
| | | | MM:TM | | DM:TM | | | | | |
| | | Polymer | (40:60)^{c} | | (40:60)^{d} | | AMB^{e} | | FLU^{f} | |
| Isolate | Trial^{a} | batches^{b} | MIC^{g} | MFC^{h} | MIC^{g} | MFC^{h} | MIC^{g} | MFC^{h} | MIC^{g} | MFC^{h} |
| C. albicans | 1 | 1, 9 | 15.2 | 15.2 | 13.2 | 13.2 | 0.9 | 1.7 | 0.7 | >653 |
| (SC5314) | 2 | 4, 10 | 15.2 | 30.3 | 13.2 | 13.2 | 0.9 | 1.7 | 0.7 | >653 |
| | avg. | | 15.2 | 22.7 | 13.2 | 13.2 | 0.9 | 1.7 | 0.7 | >653 |
| C. lusitaniae | 1 | 1, 9 | 3.8 | 7.6 | 3.3 | 6.6 | 0.4 | 1.7 | 2.6 | 163.3 |
| (CL3) | 2 | 4, 10 | 3.8 | 7.6 | 6.6 | 9.9 | 0. | 1.7 | 2.6 | 326.5 |
| | avg. | | 3.8 | 7.6 | 4.9 | 8.2 | 0.4 | 1.7 | 2.6 | 244.9 |
| C. albicans | 1 | 1, 9 | 15.2 | 30.3 | 13.2 | 26.3 | 1.7 | 3.4 | >653 | >653 |
| (K1) | 2 | 4, 10 | 30.3 | 30.3 | 26.3 | 26.3 | 1.7 | 3.4 | >653 | >653 |
| | avg. | | 22.7 | 30.3 | 19.7 | 26.3 | 1.7 | 3.4 | >653 | >653 |
| C. albicans | 1 | 1, 9 | 15.2 | 30.3 | 13.2 | 13.2 | 0.9 | 1.7 | >653 | >653 |
| (Gu5) | 2 | 4, 10 | 15.2 | 30.3 | 13.2 | 26.3 | 0.9 | 1.7 | >653 | >653 |
| | avg. | | 15.2 | 30.3 | 13.2 | 19.7 | 0.9 | 1.7 | >653 | >653 |
| C. albicans | 1 | 1, 9 | 15.2 | 30.3 | 13.2 | 13.2 | 3.4 | 13.5 | >653 | >653 |
| (E4) | 2 | 4, 10 | 15.2 | 30.3 | 13.2 | 26.3 | 3.4 | 13.5 | >653 | >653 |
| | avg. | | 15.2 | 30.3 | 13.2 | 19.7 | 3.4 | 13.5 | >653 | >653 |

^{a}Each experiment was repeated in duplicate on separate days in at least two different trial experiments
^{b}See Tables 6 and 7 for polymer batch characterization
^{c}Average molecular weight of MM:TM copolymer used for molarity conversion is 3300 g/mol
^{d}Average molecular weight of DM:TM copolymer used for molarity conversion is 3800 g/mol
^{e}Amphotericin B (AMB), molecular weight = 924.091 g/mol.
^{f}Fluconazole (FLU), molecular weight = 306.271 g/mol.
^{g}Minimum inhibitory concentration (MIC) as determined by OD₆₀₀ measurements after 48 hours.
^{h}Minimum fungicidal concentration (MFC).

TABLE 12
Candida albicans synergy checkerboard results with fluconazole and
MM:TM (μg/mL) with an inoculum concentration of 1.25 × 10⁵ cells/mL at 30° C.

| | | | MIC | MIC | | MIC | | | | |
|---|---|---|---|---|---|---|---|---|---|---|
| | | | polymer | polymer | | azole | MIC azole | | | |
| | | Polymer | alone | combo | | alone | combination | | ΣFIC Index | |
| Isolate | Trial^{a} | batch^{b} | (μg/mL)^{c} | (μg/mL)^{d} | FIC_{polymer}^{e} | (μg/mL)^{f} | (μg/mL)^{g} | FIC_{azole}^{h} | (interpretation)ⁱ | |
| SC5314 | 1 | 1 | >50 | <0.05 | 0.001 | <0.2 | <0.2 | 1 | 1.001 | |
| | 2 | 1 | 50 | <0.1 | 0.010 | <0.8 | <0.8 | 1 | 1.010 | |
| | 3 | 2 | 50 | <0.05 | 0.001 | <0.4 | <0.4 | 1 | 1.001 | |
| | Avg. | | 66.7 | <0.05 | 0.004 | <0.3 | <0.3 | 1 | 1.004 | (I) |
| K1 | 1 | 1 | >50 | 12.5 | 0.120 | >25 | <0.4 | 0.004 | 0.124 | |
| | 2 | 1 | 100 | 6.3 | 0.063 | >50 | <0.8 | 0.004 | 0.067 | |
| | 3 | 2 | >50 | 6.3 | 0.063 | >25 | <0.4 | 0.004 | 0.067 | |
| | Avg. | | 100 | 8.4 | 0.082 | >50 | <0.4 | 0.002 | 0.084 | (S) |

^{a}Each experiment was repeated in duplicate on separate days in at least two different trial experiments
^{b}See Tables 6 and 7 for polymer batch characterization
^{c}Minimum inhibitory concentration (MIC) as determined by OD₆₀₀ measurements after 48 hours for polymer as a single agent.
^{d}Minimum inhibitory concentration (MIC) as determined by OD₆₀₀ measurements after 48 hours for polymer in combination with fluconazole.
^{e}Fractional inhibitory concentration (FIC) for polymer
^{f}Minimum inhibitory concentration (MIC) as determined by OD₆₀₀ measurements after 48 hours for fluconazole as a single agent
^{g}Minimum inhibitory concentration (MIC) as determinedby OD₆₀₀ measurements after 48 hours for fluconazole in combination with polymer.
^{h}FIC for fluconazole.
ⁱAbbreviations for interpretations: S, synergy; A, antagonism; I, indifference

TABLE 13
Candida albicans synergy checkerboard results with Fluconazole and
MM:TM (μM) with an inoculum concentration of 1.25 × 10⁵ cells/mL at 30° C.

| | | | MIC | MIC | | MIC | MIC | | | |
|---|---|---|---|---|---|---|---|---|---|---|
| | | | polym. | polymer | | azole | azole | | | |
| | | Polymer | alone | combo | | alone | combo | | ΣFIC Index | |
| Isolate | Trial^{a} | batch^{b} | (μM)^{c} | (μM)^{d} | FIC_{polymer}^{e} | (μM)^{f} | (μM)^{g} | FIC_{azole}^{h} | (interpretation)ⁱ | |
| SC5314 | 1 | 1 | >15.2 | <0.02 | 0.001 | <0.7 | <0.7 | 1 | 1.001 | |
| | 2 | 1 | 15.2 | <0.03 | 0.010 | <2.6 | <2.6 | 1 | 1.010 | |
| | 3 | 2 | 15.2 | <0.02 | 0.001 | <1.3 | <1.3 | 1 | 1.001 | |
| | Avg. | | 20.2 | <0.02 | 0.004 | <1.0 | <1.0 | 1 | 1.004 | (I) |
| K1 | 1 | 1 | >15.2 | 3.8 | 0.120 | >81.6 | <1.3 | 0.004 | 0.124 | |
| | 2 | 1 | 30.3 | 1.9 | 0.063 | >163.3 | <2.6 | 0.004 | 0.067 | |
| | 3 | 2 | >15.2 | 1.9 | 0.063 | >81.6 | <1.3 | 0.004 | 0.067 | |
| | Avg. | | 30.3 | 2.6 | 0.082 | >163.3 | <1.3 | 0.002 | 0.084 | (S) |

^{a}Each experiment was repeated in duplicate on separate days in at least two different trial experiments.
^{b}See Table 6 for polymer batch characterization. Average molecular weight of MM:TM copolymer used for molarity conversion is 3300 g/mol. The molecular weight of fluconazole used for molarity conversion is 306.271 g/mol.
^{c}Minimum inhibitory concentration (MIC) as determined by OD₆₀₀ measurements after 48 hours for polymer as a single agent.
^{d}Minimum inhibitory concentration (MIC) as determined by OD₆₀₀ measurements after 48 hours for polymer in combination with fluconazole.
^{e}Fractional inhibitory concentration (FIC) for polymer
^{f}Minimum inhibitory concentration (MIC) as determined by OD₆₀₀ measurements after 48 hours for fluconazole as a single agent
^{g}Minimum inhibitory concentration (MIC) as determined by OD₆₀₀ measurements after 48 hours for fluconazole in combination with polymer.
^{h}FIC for fluconazole.
ⁱAbbreviations for interpretations: S, synergy; A, antagonism; I, indifference

Cryptococcus spp. Antifungal Studies:

TABLE 14
Cryptococcus spp. MIC Results by Broth Microdilution (μg/mL) with an
inoculum concentration of 1.25 × 10³ cells/mL at 30° C.

| | | | Polymer Concentration (μg/mL) | | | | | | | |
|---|---|---|---|---|---|---|---|---|---|---|
| | | | MM:TM | | DM:TM | | | | | |
| | | Polymer | (40:60) | | (40:60) | | AMB^{c} | | FLU^{d} | |
| Isolate | Trial^{a} | Batch^{b} | MIC^{e} | MFC^{f} | MIC^{e} | MFC^{f} | MIC^{e} | MFC^{f} | MIC^{e} | MFC^{f} |
| C. neoformans | 1 | 5, 12 | 1.6 | 1.6 | 3.1 | 3.1 | 0.2 | 0.4 | 1.6 | 100 |
| (JEC21) | | | | | | | | | | |
| C. neoformans | 1 | 5, 12 | 3.1 | 3.1 | 3.1 | 3.1 | 0.8 | 0.8 | 1.6 | 50 |
| (B3501) | | | | | | | | | | |
| C. neoformans | 1 | 5, 12 | 3.1 | 3.1 | 3.1 | 6.3 | 1.6 | 1.6 | 3.1 | 50 |
| (H99) | | | | | | | | | | |
| C. neoformans | 1 | 5, 12 | 3.1 | 3.1 | 3.1 | 6.3 | 0.8 | 0.8 | 1.6 | 1.6 |
| (C21F3) | | | | | | | | | | |
| C. gattii | 1 | 5, 12 | 3.1 | 3.1 | 6.3 | 12.5 | 0.8 | 3.1 | 0.8 | >100 |
| (WM276) | | | | | | | | | | |
| C. gattii | 1 | 5, 12 | 3.1 | 4.7 | 6.3 | 6.3 | ≤0.2 | 0.8 | 1.6 | 37.5 |
| (C751) | | | | | | | | | | |

^{a}Each experiment was repeated in duplicate
^{b}See Tables 6 and 7 for polymer batch characterization
^{c}Amphotericin B (AMB)
^{d}Fluconazole (FLU)
^{e}Minimum inhibitory concentration (MIC) as determined by OD₆₀₀ measurements after 48 hours.
^{f}Minimum fungicidal concentration (MFC).

TABLE 15
Cryptococcus spp. MIC Results by Broth Microdilution (μM) with an
inoculum concentration of 1.25 × 10³ cells/mL at 30° C.

| | | | Polymer Concentration (μM) | | | | | | | |
|---|---|---|---|---|---|---|---|---|---|---|
| | | | MM:TM | | DM:TM | | | | | |
| | | Polymer | (40:60)^{c} | | (40:60)^{d} | | AMB^{e} | | FLU^{f} | |
| Isolate | Trial^{a} | Batch^{b} | MIC^{g} | MFC^{h} | MIC^{g} | MFC^{h} | MIC^{g} | MFC^{h} | MIC^{g} | MFC^{h} |
| C. neoformans | 1 | 5, 12 | 0.5 | 0.5 | 0.8 | 0.8 | 0.2 | 0.4 | 5.2 | 326.5 |
| (JEC21) | | | | | | | | | | |
| C. neoformans | 1 | 5, 12 | 0.9 | 0.9 | 0.8 | 0.8 | 0.9 | 0.9 | 5.2 | 163.3 |
| (B3501) | | | | | | | | | | |
| C. neoformans | 1 | 5, 12 | 0.9 | 0.9 | 0.8 | 1.7 | 1.7 | 1.7 | 10.1 | 163.3 |
| (H99) | | | | | | | | | | |
| C. neoformans | 1 | 5, 12 | 0.9 | 0.9 | 0.8 | 1.7 | 0.9 | 0.9 | 5.2 | 5.2 |
| (C21F3) | | | | | | | | | | |
| C. gattii | 1 | 5, 12 | 0.9 | 0.9 | 1.7 | 3.3 | 0.9 | 3.4 | 2.6 | >326.5 |
| (WM276) | | | | | | | | | | |
| C. gattii | 1 | 5, 12 | 0.9 | 1.4 | 1.7 | 1.7 | ≤0.2 | 0.9 | 5.2 | 122.4 |
| (C751) | | | | | | | | | | |

^{a}Each experiment was repeated in duplicate
^{b}See Tables 6 and 7 for polymer batch characterization
^{c}Average molecular weight of MM:TM copolymer used for molarity conversion is 3300 g/mol
^{d}Average molecular weight of DM:TM copolymer used for molarity conversion is 3800 g/mol
^{e}Amphotericin B (AMB), molecular weight = 924.091 g/mol.
^{f}Fluconazole (FLU), molecular weight = 306.271 g/mol.
^{g}Minimum inhibitory concentration (MIC) as determined by OD₆₀₀ measurements after 48 hours.
^{h}Minimum fungicidal concentration (MFC).

TABLE 16
Cryptococcus spp. MIC Results by Broth Microdilution (μg/mL) with an
inoculum concentration of 1.25 × 10⁵ cells/mL at 30° C.

| | | | Polymer Concentration (μg/mL) | | | | | | | |
|---|---|---|---|---|---|---|---|---|---|---|
| | | | MM:TM | | DM:TM | | | | | |
| | | Polymer | (40:60) | | (40:60) | | AMB^{c} | | FLU^{d} | |
| Isolate | Trial^{a} | Batch^{b} | MIC^{e} | MFC^{f} | MIC^{e} | MFC^{f} | MIC^{e} | MFC^{f} | MIC^{e} | MFC^{f} |
| C. neoformans | 1 | 1, 9 | 6.3 | 6.3 | 6.3 | 12.5 | 0.8 | 3.1 | 6.3 | 50 |
| (H99) | 2 | 4, 10 | 6.3 | 12.5 | 6.3 | 12.5 | 0.8 | 3.1 | 6.3 | 50 |
| | Avg. | | 6.3 | 9.4 | 6.3 | 12.5 | 0.8 | 3.1 | 6.3 | 50 |

^{a}Each experiment was repeated in duplicate on separate days in at least two different trial experiments
^{b}See Tables 6 and 7 for polymer batch characterization
^{c}Amphotericin B (AMB)
^{d}Fluconazole (FLU)
^{e}Minimum inhibitory concentration (MIC) as determined by OD₆₀₀ measurements after 48 hours.
^{f}Minimum fungicidal concentration (MFC).

TABLE 17
Cryptococcus spp. MIC Results by Broth Microdilution (μM) with an
inoculum concentration of 1.25 × 10⁵ cells/mL at 30° C.

| | | | Polymer Concentration (μM) | | | | | | | |
|---|---|---|---|---|---|---|---|---|---|---|
| | | | MM:TM | | DM:TM | | | | | |
| | | Polymer | (40:60)^{c} | | (40:60)^{c} | | AMB^{e} | | FLU^{f} | |
| Isolate | Trial^{a} | Batch^{h} | MIC^{g} | MFC^{h} | MIC^{g} | MFC^{h} | MIC^{g} | MFC^{h} | MIC^{g} | MFC^{h} |
| C. neoformans | 1 | 1, 9 | 1.9 | 1.9 | 1.7 | 3.3 | 0.9 | 3.4 | 20.6 | 163.3 |
| (H99) | 2 | 4, 10 | 1.9 | 3.8 | 1.7 | 3.3 | 0.9 | 3.4 | 20.6 | 163.3 |
| | Avg. | | 1.9 | 2.8 | 1.7 | 3.3 | 0.9 | 3.4 | 20.6 | 163.3 |

^{a}Each experiment was repeated in duplicate on separate days in at least two different trial experiments
^{b}See Tables 6 and 7 for polymer batch characterization
^{c}Average molecular weight of MM:TM copolymer used for molarity conversion is 3300 g/mol
^{d}Average molecular weight of DM:TM copolymer used for molarity conversion is 3800 g/mol
^{e}Amphotericin B (AMB), molecular weight = 924.091 g/mol.
^{f}Fluconazole (FLU), molecular weight = 306.271 g/mol.
^{g}Minimum inhibitory concentration (MIC) as determined by OD₆₀₀ measurements after 48 hours.
^{h}Minimum fungicidal concentration (MFC).

TABLE 18
Cryptococcus neoformans synergy checkerboard results with AMB and
MM:TM (μg/mL) with an inoculum concentration of 1.25 × 10⁵ cells/mL at 30° C.

| | | | MIC | MIC | | MIC | MIC | | | |
|---|---|---|---|---|---|---|---|---|---|---|
| | | | polymer | polymer | | AMB | AMB | | | |
| | | Polymer | alone | combo | | alone | combo | | ΣFIC Index | |
| Isolate | Trial^{a} | batch^{b} | (μg/mL)^{c} | (μg/mL)^{d} | FIC_{polymer}^{e} | (μg/mL)^{f} | (μg/mL)^{g} | FIC_{AMB}^{h} | (interpretation)ⁱ | |
| H99 | 1 | 1 | 12.5 | 0.03 | 0.002 | 3.1 | 0.2 | 0.06 | 0.09 | |
| | 2 | 1 | 6.3 | 0.03 | 0.005 | 3.1 | 0.1 | 0.03 | 0.08 | |
| | Avg. | | 9.4 | 0.03 | 0.003 | 3.1 | 0.15 | 0.05 | 0.08 | (S) |

^{a}Each experiment was repeated in duplicate on separate days in at least two different trial experiments
^{b}See Table 6 for polymer batch characterization.
^{c}Minimum inhibitory concentration (MIC) as determined by OD₆₀₀ measurements after 48 hours for polymer as a single agent.
^{d}Minimum inhibitory concentration (MIC) as determined by OD₆₀₀ measurements after 48 hours for polymer in combination with amphotericin B(AMB).
^{e}Fractional inhibitory concentration (FIC) for polymer
^{f}Minimum inhibitory concentration (MIC) as determined by OD₆₀₀ measurements after 48 hours for AMB as a single agent
^{g}Minimum inhibitory concentration (MIC) as determined by OD₆₀₀ measurements after 48 hours for AMB in combination with polymer.
^{h}FIC for AMB.
ⁱAbbreviations for interpretations: S, synergy; A, antagonism; I, indifference

TABLE 19
Cryptococcus neoformans synergy checkerboard results with AMB and
MM:TM (μM) with an inoculum concentration of 1.25 × 10⁵ cells/mL at 30° C.

| | | | MIC | MIC | | MIC | MIC | | | |
|---|---|---|---|---|---|---|---|---|---|---|
| | | | polymer | polymer | | AMB | AMB | | | |
| | | Polymer | alone | combination | | alone | combination | | ΣFIC Index | |
| Isolate | Trial^{a} | batch^{b} | (μM)^{c} | (μM)^{d} | FIC_{polymer}^{e} | (μM)^{f} | (μM)^{g} | FIC_{AMB}^{h} | (interpretation)ⁱ | |
| H99 | 1 | 1 | 3.8 | 0.009 | 0.002 | 3.4 | 0.2 | 0.06 | 0.09 | |
| | 2 | 1 | 1.9 | 0.009 | 0.005 | 3.4 | 0.1 | 0.03 | 0.08 | |
| | Avg. | | 2.8 | 0.009 | 0.003 | 3.4 | 0.16 | 0.05 | 0.08 | (S) |

^{a}Each experiment was repeated in duplicate on separate days in at least two different trial experiments.
^{b}See Table 6 for polymer batch characterization. Average molecular weight of MM:TM copolymer used for molarity conversion is 3300 g/mol. Amphotericin B (AMB) molecular weight used for molarity conversion is 924.091 g/mol.
^{c}Minimum inhibitory concentration (MIC) as determined by OD₆₀₀ measurements after 48 hours for polymer as a single agent.
^{d}Minimum inhibitory concentration (MIC) as determined by OD₆₀₀ measurements after 48 hours for polymer in combination with AMB.
^{e}Fractional inhibitory concentration (FIC) for polymer
^{f}Minimum inhibitory concentration (MIC) as determined by OD₆₀₀ measurements after 48 hours for AMB as a single agent
^{g}Minimum inhibitory concentration (MIC) as determined by OD₆₀₀ measurements after 48 hours for AMB in combination with polymer.
^{h}FIC for AMB.
ⁱAbbreviations for interpretations: S, synergy; A, antagonism; I, indifference

Aspergillus spp. Antifungal Studies:

TABLE 20
Aspergillus spp. MIC Results by Broth Microdilution (μg/mL)
with an inoculum concentration of 1 × 10⁵ cells/mL at 35° C.

| | | | MIC (μg/mL)^{c} | | | |
|---|---|---|---|---|---|---|
| | | Polymer | MM:TM | DM:TM | | |
| Isolate | Trial^{a} | Batch^{b} | (40:60) | (40:60) | POS^{d} | ITRA^{e} |
| A. terreus | 1 | 3, 9 | >200 | >200 | 1.0 | 1.0 |
| NIH2624 | | | | | | |
| A. | 1 | 3, 9 | >200 | >200 | 2.0 | 1.0 |
| fumigatus | | | | | | |
| AF293 | | | | | | |
| A. | 1 | 3, 9 | >200 | >200 | 0.5 | >1.0 |
| fumigatus | | | | | | |
| CEA10 | | | | | | |
| A. | 1 | 3, 9 | >200 | >200 | >32 | 48.0 |
| fumigatus | | | | | | |
| F11628 | | | | | | |
| A. | 1 | 3, 9 | >200 | >200 | 1.7 | 32 |
| fumigatus | | | | | | |
| F16216 | | | | | | |

^{a}Each experiment was repeated in duplicate
^{b}See Tables 6 and 7 for polymer batch characterization
^{c}Minimum inhibitory concentration (MIC) as determined visually under dissecting scope after 48 hours
^{d}Posaconazole (POS)
^{e}Itraconazole (ITRA).

TABLE 21
Aspergillus spp. MIC Results by Broth Microdilution (μM) with
an inoculum concentration of 1 × 10⁵ cells/mL at 35° C.

| | | | MIC (μM)^{c} | | | |
|---|---|---|---|---|---|---|
| | | Polymer | MM:TM | DM:TM | | |
| Isolate | Trial^{a} | Batch^{b} | (40:60)^{d} | (40:60)^{e} | POS^{f} | ITRA^{g} |
| A. terreus | 1 | 3, 9 | >60.6 | >52.6 | 1.4 | 1.4 |
| NIH2624 | | | | | | |
| A. fumigatus | 1 | 3, 9 | >60.6 | >52.6 | 2.9 | 1.4 |
| AF293 | | | | | | |
| A. fumigatus | 1 | 3, 9 | >60.6 | >52.6 | 0.7 | >1.4 |
| CEA10 | | | | | | |
| A. fumigatus | 1 | 3, 9 | >60.6 | >52.6 | >45.7 | 68.5 |
| F11628 | | | | | | |
| A. fumigatus | 1 | 3, 9 | >60.6 | >52.6 | 2.5 | 45.3 |
| F16216 | | | | | | |

^{a} Each experiment was repeated in duplicate
^{b}See Tables 6 and 7 for polymer batch characterization
^{c}Minimum inhibitory concentration (MIC) as determined visually under dissecting scope after 48 hours.
^{d}Average molecular weight of MM:TM copolymer used for molarity conversion is 3300 g/mol
^{e}Average molecular weight of DM:TM copolymer used for molarity conversion is 3800 g/mol
^{f}Posaconazole (POS), molecular weight = 700.778 g/mol
^{e}Itraconazole (ITRA), molecular weight = 705.64.

TABLE 22
Aspergillus fumigatus synergy checkerboard results with POS and MM:TM
(μg/mL) with an inoculum concentration of 1.25 × 10⁵ cells/mL at 35° C.

| | | | MIC | | | MIC | | | |
|---|---|---|---|---|---|---|---|---|---|
| | | | polymer | MIC polymer | | azole | MIC azole | | |
| | | Polymer | alone | combination | | alone | combination | | ΣFIC Index |
| Isolate | Trial^{a} | batch^{b} | (μg/mL)^{c} | (μg/mL)^{d} | FIC_{polymer}^{e} | (μg/mL)^{f} | (μg/mL)^{g} | FIC_{azole}^{h} | (interpretation)ⁱ |
| AF293 | 1 | 1 | >100 | 6.25 | 0.032 | >1 | 0.42 | 0.21 | 0.242 |
| | 2 | 3 | >100 | 1.56 | 0.008 | 0.24 | 0.06 | 0.25 | 0.258 |
| CEA10 | Avg. | | >100 | 3.91 | 0.020 | 1.12 | 0.24 | 0.21 | 0.234 (S) |
| | 1 | 1 | >100 | 1.56 | 0.008 | 0.75 | 0.13 | 0.173 | 0.181 |
| | 2 | 3 | >100 | 1.56 | 0.008 | 0.24 | 0.06 | 0.250 | 0.258 |
| F11628 | Avg | | >100 | 1.56 | 0.008 | 0.50 | 0.10 | 0.200 | 0.208 (S) |
| | 1 | 1 | >100 | 1.56 | 0.008 | 48 | 0.09 | 0.002 | 0.010 |
| | 2 | 3 | >100 | 6.25 | 0.031 | >48 | 0.06 | 0.001 | 0.032 |
| F16216 | Avg. | | >100 | 3.91 | 0.020 | 72 | 0.08 | 0.001 | 0.021 (S) |
| | 1 | 1 | >100 | 1.56 | 0.008 | 2 | 1.5 | 0.75 | 0.758 |
| | 2 | 3 | >100 | 1.56 | 0.008 | 1.5 | 0.46 | 0.31 | 0.318 |
| | Avg. | | >100 | 1.56 | 0.008 | 1.75 | 0.98 | 0.560 | 0.5668 (I) |

^{a}Each experiment was repeated in duplicate on separate days in at least two different trial experiments.
^{b}See Table 6 for polymer batch characterization.
^{c}Minimum inhibitory concentration (MIC) for polymer as a single agent.
^{d}Minimum inhibitory concentration (MIC) after 48 hours for polymer in combination with posaconazole (POS).
^{e}Fractional inhibitory concentration (FIC) for polymer
^{f}Minimum inhibitory concentration (MIC) after 48 hours for POS as a single agent
^{g}Minimum inhibitory concentration (MIC) after 48 hours for POS in combination with polymer.
^{h}FIC for POS.
ⁱAbbreviations for interpretations:
S, synergy;
A, antagonism;
I, indifference

TABLE 23
Aspergillus fumigatus synergy checkerboard results with POS and MM:TM
(μM) with an inoculum concentration of 1.25 × 10⁵ cells/mL at 35° C.

| | | | MIC | | | MIC | | | |
|---|---|---|---|---|---|---|---|---|---|
| | | | polymer | MIC polymer | | azole | MIC azole | | |
| | | Polymer | alone | combination | | alone | combination | | ΣFIC Index |
| Isolate | Trial^{a} | batch^{b} | (μM)^{c} | (μM)^{d} | FIC_{polymer}^{e} | (μM)^{f} | (μM)^{g} | FIC_{azole}^{h} | (interpretation)ⁱ |
| AF293 | 1 | 1 | >30.3 | 0.18 | 0.032 | >1.43 | 0.60 | 0.21 | 0.242 |
| | 2 | 3 | >30.3 | 0.47 | 0.008 | 0.34 | 0.09 | 0.25 | 0.258 |
| | Avg. | | >30.3 | 1.18 | 0.020 | 1.60 | 0.34 | 0.21 | 0.234 (S) |
| CEA10 | 1 | 1 | >30.3 | 0.47 | 0.008 | 1.07 | 0.19 | 0.173 | 0.181 |
| | 2 | 3 | >30.3 | 0.47 | 0.008 | 0.34 | 0.09 | 0.250 | 0.258 |
| | Avg | | >30.3 | 0.47 | 0.008 | 0.71 | 0.14 | 0.200 | 0.208 (S) |
| F11628 | 1 | 1 | >30.3 | 0.47 | 0.008 | 68.50 | 0.13 | 0.002 | 0.010 |
| | 2 | 3 | >30.3 | 1.89 | 0.031 | >68.50 | 0.09 | 0.001 | 0.032 |
| | Avg. | | >30.3 | 1.18 | 0.020 | 102.74 | 0.11 | 0.001 | 0.021 (S) |
| F16216 | 1 | 1 | >30.3 | 0.47 | 0.008 | 2.85 | 2.14 | 0.75 | 0.758 |
| | 2 | 3 | >30.3 | 0.47 | 0.008 | 2.14 | 0.66 | 0.31 | 0.318 |
| | Avg. | | >30.3 | 0.47 | 0.008 | 2.50 | 1.40 | 0.560 | 0.5668 (I) |

^{a}Each experiment was repeated in duplicate on separate days in at least two different trial experiments.
^{b}See Table 6 for polymer batch characterization. Average molecular weight of MM:TM copolymer used for molarity conversion is 3300 g/mol. The molecular weight of posaconazole (POS) used for molarity conversion is 700.778 g/mol.
^{c}Minimum inhibitory concentration (MIC) after 48 hours for polymer as a single agent.
^{d}Minimum inhibitory concentration (MIC) after 48 hours for polymer in combination with POS.
^{e}Fractional inhibitory concentration (FIC) for polymer
^{f}Minimum inhibitory concentration (MIC) after 48 hours for POS as a single agent
^{g}Minimum inhibitory concentration (MIC) after 48 hours for POS in combination with polymer.
^{h}FIC for POS.
ⁱAbbreviations for interpretations:
S, synergy;
A, antagonism;
I, indifference

TABLE 24
Aspergillus fumigatus synergy checkerboard results with ITRA and MM:TM
(μg/mL) with an inoculum concentration of 1.25 × 10⁵ cells/mL at 35° C.

| | | | MIC | | | MIC | | | |
|---|---|---|---|---|---|---|---|---|---|
| | | | polymer | MIC polymer | | azole | MIC azole | | |
| | | Polymer | alone | combination | | alone | combination | | ΣFIC Index |
| Isolate | Trial^{a} | batch^{b} | (μg/mL)^{c} | (μg/mL)^{d} | FIC_{polymer}^{e} | (μg/mL)^{f} | (μg/mL)^{g} | FIC_{azole}^{h} | (interpretation)ⁱ |
| AF293 | 1 | 1 | >100 | 6.25 | 0.032 | 2 | 0.42 | 0.210 | 0.242 |
| | 2 | 3 | >100 | 1.56 | 0.008 | 0.42 | 0.06 | 0.143 | 0.151 |
| | Avg. | | >100 | 3.91 | 0.020 | 1.21 | 0.24 | 0.198 | 0.218 (S) |
| CEA10 | 1 | 1 | >100 | 1.56 | 0.008 | >1 | 0.32 | 0.160 | 0.168 |
| | 2 | 3 | >100 | 1.56 | 0.008 | >1 | 0.08 | 0.040 | 0.048 |
| | Avg | | >100 | 1.56 | 0.008 | >1 | 0.20 | 0.100 | 0.108 (S) |
| F11628 | 1 | 1 | >100 | 1.56 | 0.008 | 48 | 0.75 | 0.016 | 0.024 |
| | 2 | 3 | >100 | 12.5 | 0.063 | >48 | 0.06 | 0.001 | 0.064 |
| | Avg. | | >100 | 7.0 | 0.035 | 72 | 0.41 | 0.006 | 0.041 (S) |
| F16216 | 1 | 1 | >100 | 1.56 | 0.008 | 32 | 24 | 0.750 | 0.758 |
| | 2 | 3 | >100 | >100 | 1.000 | >48 | >48 | 1.000 | 2.000 |
| | Avg. | | >100 | 100.78 | 0.504 | 64 | 60 | 0.938 | 1.442 (I) |

^{a}Each experiment was repeated in duplicate on separate days in at least two different trial experiments.
^{b}See Table 6 for polymer batch characterization.
^{c}Minimum inhibitory concentration (MIC) after 48 hours for polymer as a single agent.
^{d}Minimum inhibitory concentration (MIC) after 48 hours for polymer in combination with itraconazole (ITRA).
^{e}Fractional inhibitory concentration (FIC) for polymer
^{f}Minimum inhibitory concentration (MIC) after 48 hours for ITRA as a single agent
^{g}Minimum inhibitory concentration (MIC after 48 hours for ITRA in combination with polymer.
^{h}FIC for ITRA.
ⁱAbbreviations for interpretations:
S, synergy;
A, antagonism;
I, indifference

TABLE 25
Aspergillus fumigatus synergy checkerboard results with ITRA and MM:TM
(μM) with an inoculum concentration of 1.25 × 10⁵ cells/mL at 35° C.

| | | | MIC | | | MIC | | | |
|---|---|---|---|---|---|---|---|---|---|
| | | | polymer | MIC polymer | | azole | MIC azole | | |
| | | Polymer | alone | combination | | alone | combination | | ΣFIC Index |
| Isolate | Trial^{a} | batch^{b} | (μM)^{c} | (μM)^{d} | FIC_{polymer}^{e} | (μM)^{f} | (μM)^{g} | FIC_{azole}^{h} | (interpretation)ⁱ |
| AF293 | 1 | 1 | >30.3 | 1.89 | 0.032 | 2.83 | 0.60 | 0.210 | 0.242 |
| | 2 | 3 | >30.3 | 0.47 | 0.008 | 0.60 | 0.09 | 0.143 | 0.151 |
| | Avg. | | >30.3 | 1.18 | 0.020 | 1.71 | 0.34 | 0.198 | 0.218 (S) |
| CEA10 | 1 | 1 | >30.3 | 0.47 | 0.008 | >1.42 | 0.45 | 0.160 | 0.168 |
| | 2 | 3 | >30.3 | 0.47 | 0.008 | >1.42 | 0.11 | 0.040 | 0.048 |
| | Avg | | >30.3 | 0.47 | 0.008 | >1.42 | 0.28 | 0.100 | 0.108 (S) |
| F11628 | 1 | 1 | >30.3 | 0.47 | 0.008 | 68.02 | 1.06 | 0.016 | 0.024 |
| | 2 | 3 | >30.3 | 3.79 | 0.063 | >68.02 | 0.09 | 0.001 | 0.064 |
| | Avg. | | >30.3 | 2.12 | 0.035 | 102.04 | 0.58 | 0.006 | 0.041 (S) |
| F16216 | 1 | 1 | >30.3 | 0.47 | 0.008 | 43.35 | 34.01 | 0.750 | 0.758 |
| | 2 | 3 | >30.3 | >30.3 | 1.000 | >68.02 | >68.02 | 1.000 | 2.000 |
| | Avg. | | >30.3 | 30.54 | 0.504 | 90.70 | 85.03 | 0.938 | 1.442 (I) |

^{a}Each experiment was repeated in duplicate on separate days in at least two different trial experiments.
^{b}See Table 6 for polymer batch characterization. Average molecular weight of MM:TM copolymer used for molarity conversion is 3300 g/mol. The molecular weight of itraconazole (ITRA) used for molarity conversion is 705.64 g/mol.
^{c}Minimum inhibitory concentration (MIC) as determined by OD₆₀₀ measurements after 48 hours for polymer as a single agent.
^{d}Minimum inhibitory concentration (MIC) as determined by OD₆₀₀ measurements after 48 hours for polymer in combination with ITRA.
^{e}Fractional inhibitory concentration (FIC) for polymer
^{f}Minimum inhibitory concentration (MIC) as determined by OD₆₀₀ measurements after 48 hours for ITRA as a single agent
^{g}Minimum inhibitory concentration (MIC) as determined by OD₆₀₀ measurements after 48 hours for ITRA in combination with polymer.
^{h}FIC for ITRA.
ⁱAbbreviations for interpretations:
S, synergy;
A, antagonism;
I, indifference

TABLE 26
Aspergillus fumigatus synergy checkerboard results with POS and MM:TM
(μg/mL) with an inoculum concentration of 1.25 × 10⁵ cells/mL at 35° C.

| | | | MIC | | | | | | |
|---|---|---|---|---|---|---|---|---|---|
| Isolate | | | polymer | MIC polymer | | MIC azole | MIC azole | | |
| (Cyp51A | | Polymer | alone | combination | | alone | combination | | ΣFIC Index |
| mutation) | Trial^{a} | batch^{b} | (μg/mL)^{c} | (μg/mL)^{d} | FIC_{polymer}^{e} | (μg/mL)^{f} | (μg/mL)^{g} | FIC_{azole}^{h} | (interpretation)ⁱ |
| 12-7505446 | 1 | 7 | >100 | 3.1 | 0.016 | 0.09 | 0.05 | 0.556 | 0.572 |
| Cyp51 | 2 | 8 | >100 | 1.6 | 0.008 | 0.19 | 0.05 | 0.263 | 0.271 |
| [TR34/L98H | Avg. | | >100 | 2.4 | 0.012 | 0.14 | 0.05 | 0.357 | 0.369 (S) |
| 12-7505220 | 1 | 7 | >100 | 3.1 | 0.016 | 0.375 | 0.09 | 0.240 | 0.256 |
| Cyp51 | 2 | 8 | >100 | 12.5 | 0.062 | 0.19 | 0.05 | 0.263 | 0.325 |
| [TR34/L98H] | Avg | | >100 | 7.8 | 0.039 | 0.283 | 0.07 | 0.247 | 0.286 (S) |
| 08-12-12-13 | 1 | 7 | >100 | >100 | 1.000 | 0.375 | 0.375 | 1.000 | 2.000 |
| Cyp51 | 2 | 8 | >100 | >100 | 1.000 | 0.09 | 0.09 | 1.000 | 2.000 |
| [TR34/L98H] | Avg. | | >100 | >100 | 1.000 | 0.232 | 0.232 | 1.000 | 2.00 (I) |
| 08-36-03-25 | 1 | 7 | >100 | 100 | 0.500 | 0.75 | 0.375 | 0.500 | 1.000 |
| Cyp51 | 2 | 8 | >100 | 100 | 0.500 | 0.19 | 0.09 | 0.474 | 0.974 |
| [TR34/L98H] | Avg. | | >100 | 100 | 0.500 | 0.47 | 0.232 | 0.495 | 0.995 (I) |
| 08-31-08-91 | 1 | 7 | >100 | 12.5 | 0.062 | 1.5 | 0.75 | 0.500 | 0.562 |
| Cyp51 | 2 | 8 | >100 | 12.5 | 0.062 | 1.5 | 0.375 | 0.250 | 0.312 |
| [TR34/L98H] | Avg. | | >100 | 12.5 | 0.062 | 1.5 | 0.562 | 0.375 | 0.437 (S) |
| 08-19-02-61 | 1 | 7 | >100 | 1.6 | 0.008 | 1.5 | 0.75 | 0.500 | 0.508 |
| Cyp51 | 2 | 8 | >100 | 1.6 | 0.008 | 0.75 | 0.05 | 0.067 | 0.075 |
| [TR34/L98H] | Avg. | | >100 | 1.6 | 0.008 | 1.125 | 0.400 | 0.356 | 0.364 (S) |
| D5 | 1 | 7 | >100 | 1.6 | 0.008 | 3 | 0.375 | 0.125 | 0.133 |
| Cyp51 [G54E] | 2 | 8 | >100 | 1.6 | 0.008 | 3 | 0.19 | 0.063 | 0.071 |
| | Avg. | | >100 | 1.6 | 0.008 | 3 | 0.282 | 0.094 | 0.102 (S) |
| D6 | 1 | 7 | >100 | 3.1 | 0.016 | 0.75 | 0.375 | 0.500 | 0.516 |
| Cyp51 [G54V] | 2 | 8 | >100 | 1.6 | 0.008 | 0.375 | 0.05 | 0.133 | 0.141 |
| | Avg. | | >100 | 2.4 | 0.012 | 0.563 | 0.213 | 0.377 | 0.389 (S) |
| D7 | 1 | 7 | >100 | 1.6 | 0.008 | 0.19 | 0.05 | 0.263 | 0.271 |
| Cyp51 [WT] | 2 | 8 | >100 | 1.6 | 0.008 | <0.05 | <0.05 | 1.000 | 1.008 |
| | Avg. | | >100 | 1.6 | 0.008 | 0.107 | 0.038 | 0.350 | 0.358 (S) |
| D9 | 1 | 7 | >100 | 25 | 0.125 | 3 | 0.75 | 0.25 | 0.375 |
| Cyp51 [M220K] | 2 | 8 | >100 | 25 | 0.125 | 24 | 0.375 | 0.016 | 0.141 |
| | Avg. | | >100 | 25 | 0.125 | 13.5 | 0.562 | 0.042 | 0.167 (S) |

^{a}Each experiment was repeated in duplicate on separate days in at least two different trial experiments.
^{b}See Table 6 for polymer batch characterization.
^{c}Minimum inhibitory concentration (MIC) as determined by OD₆₀₀ measurements after 48 hours for polymer as a single agent.
^{d}Minimum inhibitory concentration (MIC) as determined by OD₆₀₀ measurements after 48 hours for polymer in combination with posaconazole (POS).
^{e}Fractional inhibitory concentration (FIC) for polymer
^{f}Minimum inhibitory concentration (MIC) as determined by OD₆₀₀ measurements after 48 hours for POS as a single agent
^{g}Minimum inhibitory concentration (MIC) as determined by OD₆₀₀ measurements after 48 hours for POS in combination with polymer.
^{h}FIC for POS.
ⁱAbbreviations for interpretations:
S, synergy;
A, antagonism;
I, indifference

TABLE 27
Aspergillus fumigatus synergy checkerboard results with POS and MM:TM
(μM) with an inoculum concentration of 1.25 × 10⁵ cells/mL at 35° C.

| | | | MIC | | | | | | |
|---|---|---|---|---|---|---|---|---|---|
| Isolate | | | polymer | MIC polymer | | MIC azole | MIC azole | | |
| (Cyp51A | | Polymer | alone | combination | | alone | combination | | ΣFIC Index |
| mutation) | Trial^{a} | batch^{b} | (μM)^{c} | (μM)^{d} | FIC_{polymer}^{e} | (μM)^{f} | (μM)^{g} | FIC_{azole}^{h} | (interpretation)ⁱ |
| 12-7505446 | 1 | 7 | >30.3 | 0.94 | 0.016 | 0.13 | 0.07 | 0.556 | 0.572 |
| Cyp51 | 2 | 8 | >30.3 | 0.48 | 0.008 | 0.27 | 0.07 | 0.263 | 0.271 |
| [TR34/L98H | Avg. | | >30.3 | 0.73 | 0.012 | 0.20 | 0.07 | 0.357 | 0.369 (S) |
| 12-7505220 | 1 | 7 | >30.3 | 0.94 | 0.016 | 0.54 | 0.13 | 0.240 | 0.256 |
| Cyp51 | 2 | 8 | >30.3 | 3.79 | 0.062 | 0.27 | 0.07 | 0.263 | 0.325 |
| [TR34/L98H] | Avg | | >30.3 | 2.36 | 0.039 | 0.40 | 0.10 | 0.247 | 0.286 (S) |
| 08-12-12-13 | 1 | 7 | >30.3 | >30.3 | 1.000 | 0.54 | 0.54 | 1.000 | 2.000 |
| Cyp51 | 2 | 8 | >30.3 | >30.3 | 1.000 | 0.13 | 0.13 | 1.000 | 2.000 |
| [TR34/L98H] | Avg. | | >30.3 | >30.3 | 1.000 | 0.33 | 0.33 | 1.000 | 2.00 (I) |
| 08-36-03-25 | 1 | 7 | >30.3 | 30.3 | 0.500 | 1.07 | 0.54 | 0.500 | 1.000 |
| Cyp51 | 2 | 8 | >30.3 | 30.3 | 0.500 | 0.27 | 0.13 | 0.474 | 0.974 |
| [TR34/L98H] | Avg. | | >30.3 | 30.3 | 0.500 | 0.67 | 0.33 | 0.495 | 0.995 (I) |
| 08-31-08-91 | 1 | 7 | >30.3 | 3.79 | 0.062 | 2.14 | 1.07 | 0.500 | 0.562 |
| Cyp51 | 2 | 8 | >30.3 | 3.79 | 0.062 | 2.14 | 0.54 | 0.250 | 0.312 |
| [TR34/L98H] | Avg. | | >30.3 | 3.79 | 0.062 | 2.14 | 0.80 | 0.375 | 0.437 (S) |
| 08-19-02-61 | 1 | 7 | >30.3 | 0.48 | 0.008 | 2.14 | 1.07 | 0.500 | 0.508 |
| Cyp51 | 2 | 8 | >30.3 | 0.48 | 0.008 | 1.07 | 0.07 | 0.067 | 0.075 |
| [TR34/L98H] | Avg. | | >30.3 | 0.48 | 0.008 | 1.61 | 0.57 | 0.356 | 0.364 (S) |
| D5 | 1 | 7 | >30.3 | 0.48 | 0.008 | 4.28 | 0.54 | 0.125 | 0.133 |
| Cyp51 [G54E] | 2 | 8 | >30.3 | 0.48 | 0.008 | 4.28 | 0.27 | 0.063 | 0.071 |
| | Avg. | | >30.3 | 0.48 | 0.008 | 4.28 | 0.40 | 0.094 | 0.102 (S) |
| D6 | 1 | 7 | >30.3 | 0.94 | 0.016 | 1.07 | 0.54 | 0.500 | 0.516 |
| Cyp51 [G54V] | 2 | 8 | >30.3 | 0.48 | 0.008 | 0.54 | 0.07 | 0.133 | 0.141 |
| | Avg. | | >30.3 | 0.73 | 0.012 | 0.80 | 0.30 | 0.377 | 0.389 (S) |
| D7 | 1 | 7 | >30.3 | 0.48 | 0.008 | 0.27 | 0.07 | 0.263 | 0.271 |
| Cyp51 [WT] | 2 | 8 | >30.3 | 0.48 | 0.008 | <0.07 | <0.07 | 1.000 | 1.008 |
| | Avg. | | >30.3 | 0.48 | 0.008 | 0.15 | 0.05 | 0.350 | 0.358 (S) |
| D9 | 1 | 7 | >30.3 | 7.58 | 0.125 | 4.28 | 1.07 | 0.25 | 0.375 |
| Cyp51 [M220K] | 2 | 8 | >30.3 | 7.58 | 0.125 | 34.25 | 0.54 | 0.016 | 0.141 |
| | Avg. | | >30.3 | 7.58 | 0.125 | 19.26 | 0.80 | 0.042 | 0.167 (S) |

^{a}Each experiment was repeated in duplicate on separate days in at least two different trial experiments.
^{b}See Table 6 for polymer batch characterization. Average molecular weight of MM:TM copolymer used for molarity conversion is 3300 g/mol. The molecular weight of posaconazole (POS) used for molarity conversion is 700.778 g/mol.
^{c}Minimum inhibitory concentration (MIC) as determined by OD₆₀₀ measurements after 48 hours for polymer as a single agent.
^{d}Minimum inhibitory concentration (MIC) as determined by OD₆₀₀ measurements after 48 hours for polymer in combination with POS.
^{e}Fractional inhibitory concentration (FIC) for polymer
^{f}Minimum inhibitory concentration (MIC) as determined by OD₆₀₀ measurements after 48 hours for POS as a single agent
^{g}Minimum inhibitory concentration (MIC) as determined by OD₆₀₀ measurements after 48 hours for POS in combination with polymer.
^{h}FIC for POS.
ⁱAbbreviations for interpretations:
S, synergy;
A, antagonism;
I, indifference

TABLE 28
Aspergillus fumigatus synergy checkerboard results with ITRA and MM:TM
(m/mL) with an inoculum concentration of 1.25 × 10⁵ cells/mL at 35° C.

| | | | MIC | | | MIC | | | |
|---|---|---|---|---|---|---|---|---|---|
| Isolate | | | polymer | MIC polymer | | azole | MIC azole | | |
| (Cyp51A | | Polymer | alone | combination | | alone | combination | | ΣFIC Index |
| mutation) | Trial^{a} | batch^{b} | (μg/mL)^{c} | (μg/mL)^{d} | FIC_{polymer}^{e} | (μg/mL)^{f} | (μg/mL)^{g} | FIC_{azole}^{h} | (interpretation)ⁱ |
| 12-7505446 | 1 | 7 | >100 | 3.1 | 0.016 | 3 | 0.19 | 0.063 | 0.079 |
| Cyp51 | 2 | 8 | >100 | 1.6 | 0.008 | 24 | 0.75 | 0.031 | 0.039 |
| [TR34/L98H | Avg. | | >100 | 2.4 | 0.012 | 13.5 | 0.47 | 0.035 | 0.047 |
| 12-7505220 | 1 | 7 | >100 | >100 | 1.000 | >48 | >48 | 1.000 | 2.000 |
| Cyp51 | 2 | 8 | >100 | 100 | 0.500 | 12 | 3 | 0.250 | 0.750 |
| [TR34/L98H] | Avg | | >100 | 150 | 0.750 | 54 | 49.5 | 0.917 | 1.667 (I) |
| 08-12-12-13 | 1 | 7 | >100 | >100 | 1.000 | >48 | >48 | 1.000 | 2.000 |
| Cyp51 | 2 | 8 | >100 | 100 | 0.500 | 12 | 3 | 0.250 | 0.750 |
| [TR34/L98H] | Avg. | | >100 | 150 | 0.750 | 54 | 49.5 | 0.917 | 1.667 (I) |
| 08-36-03-25 | 1 | 7 | >100 | >100 | 1.000 | >48 | >48 | 1.000 | 2.000 |
| Cyp51 | 2 | 8 | >100 | >100 | 1.000 | >48 | >48 | 1.000 | 2.000 |
| [TR34/L98H] | Avg. | | >100 | >100 | 1.000 | >48 | >48 | 1.000 | 2.000 (I) |
| 08-31-08-91 | 1 | 7 | >100 | >100 | 1.000 | >48 | >48 | 1.000 | 2.000 |
| Cyp51 | 2 | 8 | >100 | >100 | 1.000 | >48 | >48 | 1.000 | 2.000 |
| [TR34/L98H] | Avg. | | >100 | >100 | 1.000 | >48 | >48 | 1.000 | 2.000 (I) |
| 08-19-02-61 | 1 | 7 | >100 | >100 | 1.000 | >48 | >48 | 1.000 | 2.000 |
| Cyp51 | 2 | 8 | >100 | 25 | 0.125 | >48 | 48 | 0.500 | 0.625 |
| [TR34/L98H] | Avg. | | >100 | 112.5 | 0.562 | >48 | 72 | 0.75 | 1.312 (I) |
| D5 | 1 | 7 | >100 | >100 | 1.000 | >48 | >48 | 1.000 | 2.000 |
| Cyp51 [G54E] | 2 | 8 | >100 | >100 | 1.000 | >48 | >48 | 1.000 | 2.000 |
| | Avg. | | >100 | >100 | 1.000 | >48 | >48 | 1.000 | 2.000 (I) |
| D6 | 1 | 7 | >100 | 100 | 0.500 | >48 | 24 | 0.250 | 0.750 |
| Cyp51 [G54V] | 2 | 8 | >100 | 12.5 | 0.062 | 48 | 0.75 | 0.016 | 0.078 |
| | Avg. | | >100 | 56.3 | 0.281 | 72 | 13.38 | 0.172 | 0.453 (S) |
| D7 | 1 | 7 | >100 | 1.6 | 0.008 | 0.75 | 0.19 | 0.253 | 0.261 |
| Cyp51 [WT] | 2 | 8 | >100 | 1.6 | 0.008 | 0.75 | 0.375 | 0.500 | 0.508 |
| | Avg. | | >100 | 1.6 | 0.008 | 0.75 | 0.283 | 0.377 | 0.385 (S) |
| D9 | 1 | 7 | >100 | >100 | 1.000 | >48 | >48 | 1.000 | 2.000 |
| Cyp51 [M220K] | 2 | 8 | >100 | >100 | 1.000 | >48 | >48 | 1.000 | 2.000 |
| | Avg. | | >100 | >100 | 1.000 | >48 | >48 | 1.000 | 2.000 (I) |

^{a}Each experiment was repeated in duplicate on separate days in at least two different trial experiments.
^{b}See Table 6 for polymer batch characterization.
^{c}Minimum inhibitory concentration (MIC) as determined by OD₆₀₀ measurements after 48 hours for polymer as a single agent.
^{d}Minimum inhibitory concentration (MIC) as determined by OD₆₀₀ measurements after 48 hours for polymer in combination with itraconazole (ITRA).
^{e}Fractional inhibitory concentration (FIC) for polymer
^{f}Minimum inhibitory concentration (MIC) as determined by OD₆₀₀ measurements after 48 hours for ITRA as a single agent
^{g}Minimum inhibitory concentration (MIC) as determined by OD₆₀₀ measurements after 48 hours for ITRA in combination with polymer.
^{h}FIC for ITRA.
ⁱAbbreviations for interpretations: S, synergy; A, antagonism; I, indifference

TABLE 29
Aspergillus fumigatus synergy checkerboard results with ITRA and MM:TM
(μM) with an inoculum concentration of 1.25 × 10⁵ cells/mL at 35° C.

| | | | MIC | | | MIC | | | |
|---|---|---|---|---|---|---|---|---|---|
| Isolate | | | polymer | MIC polymer | | azole | MIC azole | | |
| (Cyp51A | | Polymer | alone | combination | | alone | combination | | ΣFIC Index |
| mutation) | Trial^{a} | batch^{b} | (μM)^{c} | (μM)^{d} | FIC_{polymer}^{e} | (μM)^{f} | (μM)^{g} | FIC_{azole}^{h} | (interpretation)ⁱ |
| 12-7505446 | 1 | 7 | >30.3 | 0.94 | 0.016 | 4.25 | 0.27 | 0.063 | 0.079 |
| Cyp51 | 2 | 8 | >30.3 | 0.48 | 0.008 | 34.01 | 1.06 | 0.031 | 0.039 |
| [TR34/L98H | Avg. | | >30.3 | 0.73 | 0.012 | 19.13 | 0.67 | 0.035 | 0.047 |
| 12-7505220 | 1 | 7 | >30.3 | >30.3 | 1.000 | >68.02 | >68.02 | 1.000 | 2.000 |
| Cyp51 | 2 | 8 | >30.3 | 30.3 | 0.500 | 17.01 | 4.25 | 0.250 | 0.750 |
| [TR34/L98H] | Avg | | >30.3 | 45.45 | 0.750 | 76.53 | 70.15 | 0.917 | 1.667 (I) |
| 08-12-12-13 | 1 | 7 | >30.3 | >30.3 | 1.000 | >68.02 | >68.02 | 1.000 | 2.000 |
| Cyp51 | 2 | 8 | >30.3 | 30.3 | 0.500 | 17.01 | 4.25 | 0.250 | 0.750 |
| [TR34/L98H] | Avg. | | >30.3 | 45.45 | 0.750 | 76.53 | 70.15 | 0.917 | 1.667 (I) |
| 08-36-03-25 | 1 | 7 | >30.3 | >30.3 | 1.000 | >68.02 | >68.02 | 1.000 | 2.000 |
| Cyp51 | 2 | 8 | >30.3 | >30.3 | 1.000 | >68.02 | >68.02 | 1.000 | 2.000 |
| [TR34/L98H] | Avg. | | >30.3 | >30.3 | 1.000 | >68.02 | >68.02 | 1.000 | 2.000 (I) |
| 08-31-08-91 | 1 | 7 | >30.3 | >30.3 | 1.000 | >68.02 | >68.02 | 1.000 | 2.000 |
| Cyp51 | 2 | 8 | >30.3 | >30.3 | 1.000 | >68.02 | >68.02 | 1.000 | 2.000 |
| [TR34/L98H] | Avg. | | >30.3 | >30.3 | 1.000 | >68.02 | >68.02 | 1.000 | 2.000 (I) |
| 08-19-02-61 | 1 | 7 | >30.3 | >30.3 | 1.000 | >68.02 | >68.02 | 1.000 | 2.000 |
| Cyp51 | 2 | 8 | >30.3 | 7.58 | 0.125 | >68.02 | 68.02 | 0.500 | 0.625 |
| [TR34/L98H] | Avg. | | >30.3 | 34.09 | 0.562 | >68.02 | 102.04 | 0.75 | 1.312 (I) |
| D5 | 1 | 7 | >30.3 | >30.3 | 1.000 | >68.02 | >68.02 | 1.000 | 2.000 |
| Cyp51 [G54E] | 2 | 8 | >30.3 | >30.3 | 1.000 | >68.02 | >68.02 | 1.000 | 2.000 |
| | Avg. | | >30.3 | >30.3 | 1.000 | >68.02 | >68.02 | 1.000 | 2.000 (I) |
| D6 | 1 | 7 | >30.3 | 30.3 | 0.500 | >68.02 | 34.01 | 0.250 | 0.750 |
| Cyp51 [G54V] | 2 | 8 | >30.3 | 3.79 | 0.062 | 68.02 | 1.06 | 0.016 | 0.078 |
| | Avg. | | >30.3 | 17.06 | 0.281 | 102.04 | 18.96 | 0.172 | 0.453 (S) |
| D7 | 1 | 7 | >30.3 | 0.48 | 0.008 | 1.06 | 0.27 | 0.253 | 0.261 |
| Cyp51 [WT] | 2 | 8 | >30.3 | 0.48 | 0.008 | 1.06 | 0.53 | 0.500 | 0.508 |
| | Avg. | | >30.3 | 0.48 | 0.008 | 1.06 | 0.4 | 0.377 | 0.385 (S) |
| D9 | 1 | 7 | >30.3 | >30.3 | 1.000 | >68.02 | >68.02 | 1.000 | 2.000 |
| Cyp51 [M220K] | 2 | 8 | >30.3 | >30.3 | 1.000 | >68.02 | >68.02 | 1.000 | 2.000 |
| | Avg. | | >30.3 | >30.3 | 1.000 | >68.02 | >68.02 | 1.000 | 2.000 (I) |

^{a}Each experiment was repeated in duplicate on separate days in at least two different trial experiments.
^{b}See Table 6 for polymer batch characterization. Average molecular weight of MM:TM copolymer used for molarity conversion is 3300 g/mol. The molecular weight of itraconazole (ITRA) used for molarity conversion is 705.64 g/mol.
^{c}Minimum inhibitory concentration (MIC) as determined by OD₆₀₀ measurements after 48 hours for polymer as a single agent.
dMinimum inhibitory concentration (MIC) as determined by OD₆₀₀ measurements after 48 hours for polymer in combination with ITRA.
^{e}Fractional inhibitory concentration (FIC) for polymer
^{f}Minimum inhibitory concentration (MIC) as determined by OD₆₀₀ measurements after 48 hours for ITRA as a single agent
^{g}Minimum inhibitory concentration (MIC) as determined by OD₆₀₀ measurements after 48 hours for ITRA in combination with polymer.
^{h}FIC for ITRA.
ⁱAbbreviations for interpretations:
S, synergy;
A, antagonism;
I, indifference

### MM:TM Copolymer Toxicity Studies:

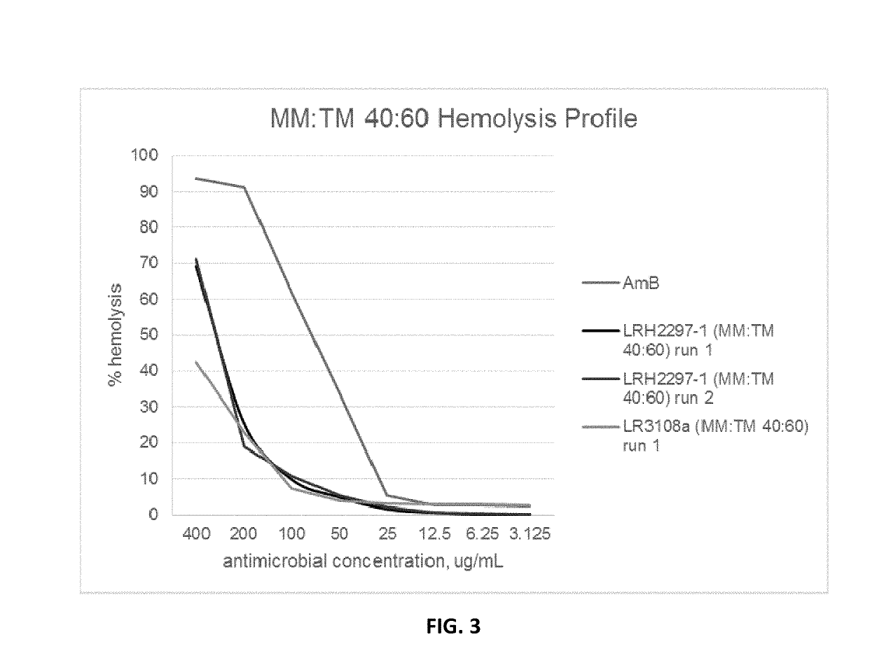

FIG. 3 is a graph depicting percent hemolysis for MM:TM 40:60 as a function of concentration. The x-axis presents the concentration of the polymers from high concentration to low concentration (that is, from 400 μg/mL to 3.125 μg/mL). The y-axis presents percent hemolysis in a standard fashion, from low to high. As can be seen from FIG. 3, the polymers tested are considerably less hemolytic at higher concentrations that is Amphotericin B (AmB). See Table 30 for details on the polymers tested.

TABLE 30
Characterization of polymers used in hemolysis studies.
Hemolysis Polymer Characterization: MM:TM

| | | | | NMR characterization^{c} | | |
|---|---|---|---|---|---|---|
| | | | | | Observed subunit | |
| | | | | | ratio | |
| | GPC characterization | | | | (cationic: | |
| Batch | PDI_{GPC} | Mn_{GPC} | DP_{GPC} | DP_{NMR} | hydrophobic) | Mn_{NMR} |
| 5 | 1.24 | 2195 | 13^{a} | 12 | 75:25 | 2596 |
| 2 | 1.18 | 4529 | 27^{b} | 14 | 79:21 | 3058 |

^{a}Side-chain protected polymer characterization by GPC using N,N-dimethylacetamide (DMAc) as the mobile phase.
^{b}Side-chain protected polymer characterization by gel permeation chromatography (GPC) using tetrahydrofuran (THF) as the mobile phase.
^{c}The degree of polymerization (DP_{NMR}) and subunit ratio for a particular polymer were calculated based on end group analysis of the tert-butyl benzoyl aromatic protons and the deprotected polymer lactam proton integration.

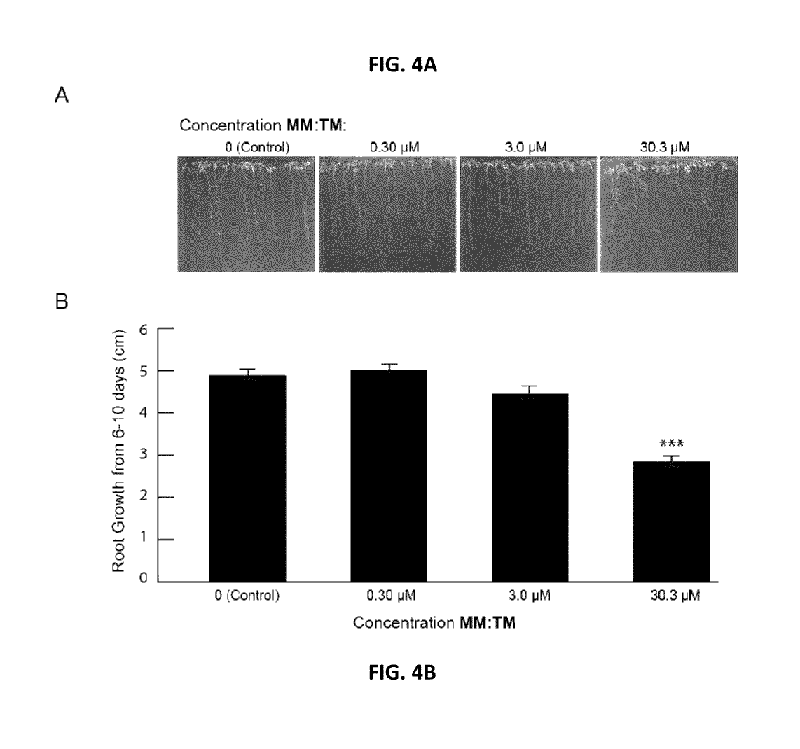

The compounds were also tested for their impact on Arabadopsis thaliana root growth. See FIGS. 4A and 4B. Root growth (in cm) was determined 6 and 10 days after germination of seeds in 0, 1, 10, and 100 μg/mL polymer. FIG. 4A shows representative images of roots after 10 days of growth. Blue dashed lines mark growth at day 6. FIG. 4B quantifies average root growth (in cm) between days 6 and 10 for each category. Data represent averages of a minimum of 37 replicates, error bars indicate standard error. Treatment groups were compared to the control for significance using a Student's t-test. N.S. indicates p>0.01, ***p=1.94e-19. See Table 31 for the characterization data on the polymers tested.

TABLE 31
Characterization of polymers used for Arabadopsis thaliana root
growth studies.
Hemolysis Polymer Characterization: MM:TM

| | | | | NMR characterization^{c} | | |
|---|---|---|---|---|---|---|
| | | | | | Observed subunit | |
| | | | | | ratio | |
| | GPC characterization | | | | (cationic: | |
| Batch | PDI_{GPC} | Mn_{GPC} | DP_{GPC} | DP_{NMR} | hydrophobic) | Mn_{NMR} |
| 6 | 1.16 | 4262 | 21 | 15 | 80:20 | 3280 |

^{a} Side-chain protected polymer characterization by GPC using N,N-dimethylacetamide (DMAc) as the mobile phase.
^{b} Side-chain protected polymer characterization by gel permeation chromatography (GPC) using tetrahydrofuran (THF) as the mobile phase.
^{c}The degree of polymerization (DP_{NMR}) and subunit ratio for a particular polymer were calculated based on end group analysis of the tert-butyl benzoyl aromatic protons and the deprotected polymer lactam proton integration.

TABLE 32
polymer efficacy against budding yeasts

| | | | Isolate | NM₂₀ | | MM:TM | | DM:TM | | POS | VOR | FLU |
|---|---|---|---|---|---|---|---|---|---|---|---|---|
| Species | Source | Description | No. | 50% | 100% | 50% | 100% | 50% | 100% | 100% | 100% | 50% |
| C. krusei | — | CLSI QC isolate for susceptibility testing | QC | 4 | 4 | 4 | 16 | 4 | 8 | — | — | 32 |
| C. albicans | Blood | ATCC90028-Reference isolate for CLSI | CA1 | 2 | 4 | 4 | 4 | 4 | 4 | — | — | 1 |
| | | susceptibility teting; also used in our murine | | | | | | | | | | |
| | | invasive candidiasis model | | | | | | | | | | |
| C. albicans | — | SC5314-Used in animal model and isolate | CA2 | 4 | 8 | 4 | 16 | 4 | 16 | — | — | 0.25 |
| | | whose genome has been sequenced; also has | | | | | | | | | | |
| | | been used in our murine invasive candidiasis | | | | | | | | | | |
| | | model | | | | | | | | | | |
| C. albicans | Blood | Azole resistant | CA3 | 4 | 8 | 16 | 16 | 16 | 64 | — | — | >64 |
| C. neoformans | | Clinical isolate; Azole susceptible | CN1 | 4 | 4 | 4 | 4 | 4 | 4 | — | — | 4 |
| C. neoformans | Popliteal | Clinical isolate (animal); Fluconazole resistant | CN2 | 2 | 4 | 4 | 4 | 4 | 4 | — | — | >64 |
| | lymph node | | | | | | | | | | | |
| C. neoformans | CSF | Clinical isolate; Fluconazole resistant | CN3 | 4 | 4 | 4 | 4 | 2 | 4 | — | — | >64 |

TABLE 33
polymer efficacy against filamentous fungi

| | | | Isolate | NM₂₀ | | MM:TM | | DM:TM | | POS | VOR | FLU |
|---|---|---|---|---|---|---|---|---|---|---|---|---|
| Species | Source | Description | No. | 50% | 100% | 50% | 100% | 50% | 100% | 100% | 100% | 50% |
| P. variotii | — | CLSI QC isolate for susceptibility testing | QC | 2 | 4 | 4 | 8 | 4 | 4 | — | 0.125 | — |
| A. fumigatus | Lung | Used in A. fumigatus genome sequencing | AF1 | 64 | >64 | 64 | >64 | 16 | >64 | — | 1 | — |
| | | project; used in our animal invasive | | | | | | | | | | |
| | | pulmonary aspergillosis models AF293 | | | | | | | | | | |
| A. fumigatus | Chest Tissue | Clinical isolate; Azole resistant | AF2 | 8 | 32 | 8 | 64 | 4 | 16 | — | 1 | — |
| A. fumigatus | Sputum | Clinical isolate; Azole susceptible | AF3 | 16 | >64 | 32 | >64 | 16 | >64 | — | 0.25 | — |
| F. oxysporum | Blood | Clinical isolate | FO1 | 4 | 4 | 4 | 8 | 4 | 8 | — | 4 | — |
| F. oxysporum | Bone | Clinical isolate | FO2 | 4 | 8 | 4 | 8 | 4 | 8 | — | 4 | — |
| F. oxysporum | Blood | Clinical isolate | FO3 | 2 | 4 | 4 | 8 | 4 | 4 | — | >16 | — |
| S. apiopsermum | Toe | Clinical isolate | SA1 | 2 | 2 | 2 | 8 | 4 | 4 | — | 1 | — |
| S. apiopsermum | Elbow Tissue | Clinical isolate | SA2 | 4 | 4 | 4 | 4 | 42 | 4 | — | 1 | — |
| L. prolificans | Chest Wound | Clinical isolate | LP1 | 4 | 4 | 4 | 4 | 4 | 4 | — | >16 | — |
| R. arrhizus | Nose Tissue | Clinical isolate | RA1 | 8 | 16 | 8 | 8 | 4 | 4 | 0.5 | — | — |
| R. arrhizus | Tissue Upper | Clinical isolate | RA2 | 8 | 8 | 8 | 8 | 4 | 8 | 0.5 | — | — |
| | Extremity | | | | | | | | | | | |
| R. arrhizus | Palate Tissue | Clinical isolate | RA3 | 8 | 8 | 8 | 8 | 4 | 8 | 0.5 | — | — |

TABLE 34
P. carinii % reduction in ATP/vehicle control

| | 24 hour | 48 hr | 72 hr |
|---|---|---|---|
| Ampicillin 10 ug/mL | 12.28 | 0 | 2.65 |
| Pentamidine 1 ug/mL | 86.79 | 87.62 | 95.23 |
| NM₂₀ 100 ug mL | 54.44 | 77.13 | 99.13 |
| 10 ug/mL | 34.01 | 28.65 | 55.91 |
| 1 ug/mL | 11.91 | 14.73 | 28.21 |
| 0.1 ug/mL | 15.54 | 14.18 | 19.79 |
| IC₅₀ | | | 3.38 ug/mL |
| MM:TM 100 ug/mL | 77.36 | 88.89 | 89.99 |
| 10 ug/mL | 49.58 | 35.99 | 43.43 |
| 1 ug/mL | 30.18 | 17.47 | 29.86 |
| 0.1 ug/mL | 18.01 | 10.80 | 23.75 |
| IC₅₀ | | | 4.49 ug/mL |
| DM:TM 100 ug/mL | 95.95 | 95.55 | 99.54 |
| 10 ug/mL | 10.78 | 36.81 | 42.82 |
| 1 ug/mL | 2.64 | 0 | 35.30 |
| 0.1 ug/mL | 0 | 0 | 0 |
| IC₅₀ | | | 4.81 ug/mL |

Tables 34 and 35 report results of assays against P. carinii and P. murina via ATP assays, respectively. To generate the data in both tables, cryopreserved and characterized P. carinii (Pc) isolated from rat lung tissue and P. murina (Pm) isolated from mouse lung tissue were distributed into triplicate wells of 48-well plates with a final volume of 500 μL and a final concentration of 5×10⁷ nuclei/mL Pc and 5×10⁶ Pm. Controls and compounds were added and incubated at 36° C., 5% CO₂. At 24, 48, and 72 hours, 10% of the well volume was removed and the ATP content was measured using Perkin Elmer ATP-liteM-brand luciferin-luciferase assay. The luminescence generated by the ATP content of the samples was measured by a BMG PolarStar optima spectrophotometer. A sample of each group was examined microscopically on the final assay day to rule out the presence of bacteria.

Calculations.

Background luminescence was subtracted and triplicate well readings of duplicate assays were averaged. For each day's reading, % reduction in ATP for all groups was calculated: (media control−experimental)/media control×100. 50% inhibitory concentration (IC₅₀) was calculated using GraphPad Prism 6 linear regression program. P. carinii and P. murina cannot be routinely cultured, Treatment is trimethoprim-sulfamethoxazole (sequentially inhibits two enzymes in folate metabolism essential for DNA synthesis). Intravenous pentamidine may be somewhat less effective than trimethoprim-sulfamethoxazole for the treatment of moderate to severe PCP.

TABLE 35
P. murina % reduction in ATP/vehicle control

| | 24 hour | 48 hr | 72 hr |
|---|---|---|---|
| Ampicillin 10 ug/mL | 0 | 0.9 | 0 |
| Pentamidine 1 ug/mL | 90.80 | 84.39 | 91.86 |
| NM₂₀ 100 ug mL | 90.37 | 93.30 | 91.82 |
| 10 ug/mL | 48.35 | 18.36 | 25.15 |
| 1 ug/mL | 26.85 | 10.90 | 0.41 |
| 0.1 ug/mL | 31.61 | 6.85 | 0 |
| IC₅₀ | | | 15.41 ug/mL |
| MM:TM 100 ug/mL | 94.54 | 93.05 | 90.65 |
| 10 ug/mL | 56.48 | 57.99 | 79.30 |
| 1 ug/mL | 28.30 | 25.4 | 17.08 |
| 0.1 ug/mL | 14.54 | 25.18 | 2.01 |
| IC₅₀ | | | 3.83 ug/mL |
| DM:TM 100 ug/mL | 94.57 | 96.00 | 97.09 |
| 10 ug/mL | 44.98 | 49.31 | 92.66 |
| 1 ug/mL | 23.32 | 20.95 | 15.31 |
| 0.1 ug/mL | 16.76 | 17.38 | 13.21 |
| IC₅₀ | | | 2.29 ug/mL |

TABLE 36
MM:TM Mice Dosage Studies
Intraperitoneal (IP): Commonly used in rats and mice IP administration results
in a faster absorption into the vasculature than subcutaneous administration
C57BL/6 mice, female, seven-weeks old, avg. weight of 20 grams
Dosage parameters: PBS control, 10 mg/kg/d, 100 mg/kg/d over 7 days
IP Injection

| | Site | Volume | | Needle site gauge | | Anesthesia required? | |
|---|---|---|---|---|---|---|---|
| recommended | Lower right | ≤40-80 mL/kg | | <22-25 g | | No | |
| | quadrant | | | | | | |
| Experimental | Lower, center | 400 uL per injection | | 22 | | No | |
| conditions | | | | | | | |
| | Expected subunit | | | | NMR characterization | | |
| | ratio (cationic: | GPC characterization | | | | Observed subunit ratio | Scale (mmol), |
| polymer | hydrophobic) | PDI_{GPC} | Mn_{GPC} | DP_{GPC} | DP_{NMR} | (cationic:hydrophobic) | yield (mg) |
| MM:TM | 40:60 | 1.21 | 4168 | 25 | 14 | 79:21 | 0.6 mmol, 53 mgs |
| MM:TM | 40:60 | 1.18 | 4191 | 25 | 15 | 80:20 | 0.6 mmol, 54 mgs |

**Polymers are TFA salts

TABLE 37
Aspergillus fumigatus synergy checkerboard results with posaconazole and MM:TM (μg/mL) with an
inoculum concentration of 1.25 × 10⁵ cells/mL.

| | | | MIC polymer | | | | | |
|---|---|---|---|---|---|---|---|---|
| | | MIC polymer | in | | MIC azole | MIC azole in | | |
| | Azole | alone | combination | | alone | combination | | ΣFIC Index |
| Isolate | Resistance | (μg/mL) | (μg/mL) | FIC_{polymer} | (μg/mL) | (μg/mL) | FIC_{azole} | (interpretation) ^{a} |
| AF293 | Senstitive | >100 | 7.0 | 0.035 | 0.5 | 0.25 | 0.500 | 0.535 (I) |
| CEA10 | Sensitive | >100 | 1.6 | 0.008 | 0.5 | 0.1 | 0.200 | 0.208 (S) |
| F11628 | Resistance | >100 | 3.9 | 0.020 | 48 | 0.1 | 0.001 | 0.021 (S) |
| F16216 | Resistance | >100 | 1.6 | 0.008 | 1.75 | 1.0 | 0.571 | 0.579 (I) |

^{a} Abbreviations for interpretations:
S, synergy;
A, antagonism;
I, indifference

TABLE 38
Aspergillus fumigatus synergy checkerboard results with Itraconazole
and MM:TM (μg/mL) with an inoculum concentration of 1.25 × 10⁵ cells/mL.

| | | | MIC polymer in | | MIC azole | MIC azole in | | |
|---|---|---|---|---|---|---|---|---|
| | Azole | MIC polymer | combination | | alone | combination | | ΣFIC Index |
| Isolate | Resistance | alone (μg/mL) | (μg/mL) | FIC_{polymer} | (μg/mL) | (μg/mL) | FIC_{azole} | (interpretation)^{a} |
| AF293 | Senstitive | >100 | 3.9 | 0.020 | 1 | 0.2 | 0.200 | 0.220 (S) |
| CEA10 | Sensitive | >100 | 1.6 | 0.008 | >1 | 0.2 | 0.100 | 0.108 (S) |
| F11628 | Resistance | >100 | 7.0 | 0.035 | 48 | 0.4 | 0.008 | 0.043 (S) |
| F16216 | Resistance | >100 | >100 | 1.000 | 32 | 32 | 1.000 | 2.000 (I) |

^{a}Abbreviations for interpretations: S, synergy: A, antagonism; I, indifference

TABLE 39
Candida albicans Synergy method results with Fluconazole and MM:TM (40:60) ^{a}

| | | MIC polymer | MIC polymer in | | MIC azole | MIC azole in | | |
|---|---|---|---|---|---|---|---|---|
| | Fluconazole | alone | combination | | alone | combination | | ΣFIC Index |
| Isolate | Resistance | (μg/mL) | (μg/mL) | FIC_{polymer} | (μg/mL) | (μg/mL) | FIC_{azole} | (interpretation) |
| K1 | Resistant | 100 | 6.25 | 0.062 | >50 | 0.4 | 0.004 | 0.066 (S) |
| SC5314 | Senstive | 50 | 0.05 | 0.001 | <0.3 | <0.3 | 1 | 1.001 (I) |

^{a} Abbreviations for interpretation: S, synergy; A, antagonism; I, indifference

CLSI M27-3A Antifungal Susceptibility testing method (RPMI 1640 0.2% glucose)
Inoculum density—1.25×10⁵ cells/mL
Incubation temperature 30° C.
Experiments repeated in duplicate on different days. All values reported represent the average MIC concentration of two trials. The average MIC concentration consistently falls within one, two-fold serial dilution of the concentration of each experimental replicate.

TABLE 40
Cryptococcus neoformens Synergy method results with Amp B and MM:TM (40:60) ^{a}

| | | MIC polymer | MIC polymer in | | MIC Amp B | MIC Amp B in | | |
|---|---|---|---|---|---|---|---|---|
| | Amp B | alone | combination | | alone | combination | | ΣFIC Index |
| Isolate | Resistance | (μg/mL) | (μg/mL) | FIC_{polymer} | (μg/mL) | (μg/mL) | FIC_{Amp B} | (interpretation) |
| H99 | Sensitive | 6.3 | 0.03 | 0.005 | 1.6 | 0.15 | 0.09 | 0.10 (S) |

^{a} Abbreviations for interpretation: S, synergy; A, antagonism; I, indifference

CLSI M27-3A Antifungal Susceptibility testing method (RPMI 1640 0.2% glucose)
Inoculum density—1.25×10⁵ cells/mL
Incubation temperature 30° C.
Experiments repeated in duplicate on different days. All values reported represent the average MIC concentration of two trials. The average MIC concentration consistently falls within one, two-fold serial dilution of the concentration of each experimental replicate.

TABLE 41
C. neoformans synergy checkerboard results with Amp B and MM:TM (μg/mL)
with an inoculum concentration of 1.25 × 10⁵ cells/mL.

| | MIC polymer | MIC polymer in | MIC Amp B | MIC Amp B in | | |
|---|---|---|---|---|---|---|
| | alone | combination | alone | combination | | ΣFIC Index |
| Isolate | (μg/mL) | (μg/mL) | (μg/mL) | (μg/mL) | FIC | (interpretation) ^{a} |
| H99 | 6.3 | 0.03 | 1.6 | 0.15 | 0.10 | S |

^{a} Abbreviations for interpretation: S, synergy; A, antagonism; I, indifference

TABLE 42
C. neoformans synergy checkerboard results with Amp B and NM₂₀ (μg/mL)
with an inoculum concentration of 1.25 × 10⁵ cells/mL.

| | MIC polymer | MIC polymer in | MIC AMB | MIC AMB in | | |
|---|---|---|---|---|---|---|
| | alone | combination | alone | combination | | ΣFIC Index |
| Isolate | (μg/mL) | (μg/mL) | (μg/mL) | (μg/mL) | FIC | (interpretation)^{a} |
| H99 | 6.3 | 0.02 | 3.1 | 0.1 | 0.033 | S |

^{a}Abbreviations for interpretation: S, synergy; A, antagonism; I, indifference

Synergism between MM:TM and NM20 with Amp B against C. neoformans is essentially identical

TABLE 43
Candida albicans synergy checkerboard results with Fluconazole and MM:TM
(μg/mL) with an inoculum concentration of 1.25 × 10⁵ cells/mL.

| | MIC polymer | MIC polymer in | MIC azole | MIC azole in | | |
|---|---|---|---|---|---|---|
| | alone | combination | alone | combination | | FIC |
| Isolate | (μg/mL) | (μg/mL) | (μg/mL) | (μg/mL) | FIC | Iinterpretation^{a} |
| K1 | 100 | 6.25 | >50 | 0.4 | 0.066 | S |
| SC5314 | 50 | 0.05 | <0.3 | <0.3 | 1.001 | I |

^{a}Abbreviations for interpretation: S, synergy; A, antagonism; I, indifference

TABLE 44
Candida albicans synergy checkerboard results with Fluconazole and NM₂₀
(μg/mL) with an inoculum concentration of 1.25 × 10⁵ cells/mL.

| | | MIC polymer in | | | | |
|---|---|---|---|---|---|---|
| | MIC polymer | combination | MIC azole | MIC azole in | | |
| | alone | (μg/mL) | alone | combination | | FIC |
| Isolate | (μg/mL) | FIC_{polymer} | (μg/mL) | (μg/mL) | FIC | interpretation |
| C. albicans | >25 | 6.3 | >25 | 0.4 | 0.13 | S |
| K1 | | | | | | |
| C. albicans | >25 | 6.3 | >25 | 0.4 | 0.13 | S |
| SC5314 | | | | | | |

^{a}Abbreviations for interpretation: S, synergy; A, antagonism; I, indifference

Synergism between MM:TM and NM20 with FLU against C. albicans have slight differences, but only due to differences in MIC azole alone

TABLE 45
Aspergillus spp. MIC Results by Broth Microdilution

| | MIC (μg/ml) | | | | | | | | | |
|---|---|---|---|---|---|---|---|---|---|---|
| | RPMI 1640 (0.2% Glucose) | | | | | RPMI 1640 (2% Glucose) | | | | |
| | MM:TM | MM:TM | DM:TM | DM:TM | | MM:TM | MM:TM | DM:TM | DM:TM | |
| Isolate | (40:60) | (50:50) | (40:60) | (50:50) | posaconazole | (40:60) | (50:50) | (40:60) | (50:50) | posaconazole |
| A. fumigatus | >200 | >200 | >200 | >200 | 2.0 | >200 | >200 | >200 | >200 | 1.0 |
| AF293 | | | | | | | | | | |
| A. fumigatus | >200 | >200 | >200 | >200 | 0.5 | >200 | >200 | >200 | >200 | 0.5 |
| CEA10 | | | | | | | | | | |
| A. fumigatus | 200 | 200 | 50 | 50 | >32 | >200 | >200 | >200 | >200 | >32 |
| F11628 | | | | | | | | | | |
| A. terreus | >200 | >200 | >200 | >200 | 1.0 | >200 | >200 | >200 | >200 | 1.0 |
| P. marneffei | 50 | 50 | 25 | 25 | 0.5 | 100 | 200 | 50 | 50 | 0.5 |
| mold | | | | | | | | | | |

EUCAST-AST-ASPERGILLUS Antifungal Susceptibility testing method (RPMI 1640 2% glucose)
Inoculum density—1.00×10⁵ cells/mL
Incubation temperature 35° C.

TABLE 46
Candida spp. MIC Results by Broth Microdilution

| | | Polymer Concentration (μg/mL) | | | | | | | | | | | |
|---|---|---|---|---|---|---|---|---|---|---|---|---|---|
| | | RPMI 1640 (0.2% Glucose) | | | | | | | | | | | |
| | | MM:TM (40:60) | | MM:TM (50:50) | | DM:TM (40:60) | | DM:TM (50:50) | | Amp B | | Fluconazole | |
| Isolate | Resistance | MIC | MFC | MIC | MFC | MIC | MFC | MIC | MFC | MIC | MFC | MIC | MFC |
| C. albicans | Fluconazole | 50 | 100 | 100 | 200 | 50 | 100 | 50 | 100 | 0.78 | 3.13 | >200 | >200 |
| (K1) | Resistant | | | | | | | | | | | | |
| C. albicans | Fluconazole | 50 | 100 | 50 | 100 | 50 | 50 | 50 | 100 | 0.78 | 1.56 | >200 | >200 |
| (Gu5) | Resistant | | | | | | | | | | | | |
| C. albicans | Amp B | 50 | 100 | 50 | 100 | 50 | 50 | 50 | 100 | 3.13 | 12.5 | >200 | >200 |
| (E4) | Resistant | | | | | | | | | | | | |
| C. albicans | Sensitive | 50 | 50 | 50 | 100 | 50 | 50 | 50 | 50 | 0.78 | 1.56 | <0.2 | >200 |
| (SCG5314) | | | | | | | | | | | | | |
| C. Lusitania | Sensitive | 12.5 | 25 | 12.5 | 25 | 12.5 | 25 | 25 | 25 | 0.4 | 1.56 | 0.78 | 50 |
| (CL3) | | | | | | | | | | | | | |

CLSI M27-3A Antifungal Susceptibility testing method (RPMI 1640 0.2% glucose)
Inoculum density—1.25×10⁵ cells/mL
incubation temperature 30° C.
   MIC studies for K1 and SC5314 isolates were repeated with an inoculum density of 1.25×10³ cells/mL and an incubation temperature of 30° C. and 37° C.

TABLE 47
Cryptococcus spp. MIC Results by Broth Microdilution

| | | Polymer Concentration (μg/mL) | | | | | | | | | | | |
|---|---|---|---|---|---|---|---|---|---|---|---|---|---|
| | | RPMI 1640 (0.2% Glucose) | | | | | | | | | | | |
| | | MM:TM (40:60) | | MM:TM (50:50) | | DM:TM (40:60) | | DM:TM (50:50) | | Amp B | | Fluconazole | |
| Isolate | Resistance | MIC | MFC | MIC | MFC | MIC | MFC | MIC | MFC | MIC | MFC | MIC | MFC |
| C. neoformans | Rapamycin | 3.1 | 3.1 | 3.1 | 3.1 | 3.1 | 6.3 | 3.1 | 3.1 | 0.8 | 0.8 | 1.6 | 1.6 |
| (C21F3) | Resistant | | | | | | | | | | | | |
| C. neoformans | Sensitive | 1.6 | 1.6 | 1.6 | 1.6 | 3.1 | 3.1 | 1.6 | 1.6 | 0.2 | 0.4 | 1.6 | 100 |
| (JEC21) | | | | | | | | | | | | | |
| C. neoformans | Sensitive | 3.1 | 3.1 | 1.6 | 3.1 | 3.1 | 3.1 | 1.6 | 1.6 | 0.8 | 0.8 | 1.6 | 50 |
| (B3501) | | | | | | | | | | | | | |
| C. neoformans | Sensitive | 3.1 | 3.1 | 1.6 | 1.6 | 3.1 | 6.3 | 1.6 | 1.6 | 1.6 | 1.6 | 3.1 | 50 |
| (H99) | | | | | | | | | | | | | |
| C. gattii | Sensitive | 3.1 | 3.1 | 3.1 | 3.1 | 6.3 | 12.5 | 3.1 | 3.1 | 0.8 | 3.1 | 0.8 | >100 |
| (WM275) | | | | | | | | | | | | | |
| C. gattii | Sensitive | 3.1 | 4.7 | 3.1 | 3.1 | 6.3 | 6.3 | 1.6 | 3.1 | ≤0.2 | 0.8 | 1.6 | 37.5 |
| (C751) | | | | | | | | | | | | | |

CLSI M27-3A Antifungal Susceptibility testing method (RPMI 1640 0.2% glucose)
Inoculum density—1.25×10³ cells/mL
Incubation temperature 37° C.
   MIC studies for H99 isolate were repeated with an inoculum density of 1.25×10⁵ cells/mL and an incubation temperature of 30° C.

TABLE 48
Fungal Strains Table

| Species | Strain name | Origin | Drug Resistances | Features/markers | Source |
|---|---|---|---|---|---|
| Candida | | | | | |
| C. albicans | SC5314 | Clinical | — | | (ATCC MYA-2876) |
| C. lusitaniae | CL3 | Clinical | — | MATalpha | (ATCC 42720) |
| C. albicans | K1 | Clinical | Fluconazole^{R} | | Gifted from Bruce Klein Lab |
| C. albicans | Gu5 | Clinical (Germany) | Fluconazole^{R} | | (ATCC MYA-574) |
| C. albicans | E4 | Clinical | Polyene antibiotics^{R} | | (ATCC 38248) |
| | | | (Amphotericin B^{R}) | | |
| Cryptococcus | | | | | |
| C. neoformans | H99 | Clinical (NY, US) | — | SeroA, MATalpha | (ATCC 208821) |
| C. neoformans | B3501 | Single progeny from cross | — | SeroD, MATalpha | (ATCC 3487) |
| | | ATCC 28957 × ATCC 28958 | | | |
| C. neoformans | JEC21 | Derived from B-3501 and B- | — | SeroD, MATalpha | (ATCC 96910) |
| | | 3502 | | | |
| C. neoformans | C21F3 | Derived from JEC 21 | rapamycin^{R} | SeroD | (ATCC MYA-737) |
| | | (ATCC 96910) | FK506^{R} | Does not express | |
| | | | | FKBP12 | |
| C. gattii | WM276 | Environmental (Sydney, AU) | — | SeroB, MATalpha | (ATCC 4071) |
| C. gatti | C751/PNG9 | Clinical (Papua New | — | SeroB, MATalpha | Hull Laboratory |
| | | Guinea) | | | |
| Aspergillus | | | | | |
| A. fumigatus | AF293 | Clinical (UK) | — | | (ATCC MYA-4609) |
| A. fumigatus | CEA10 | Clinical | — | | Keller Laboratory |
| A. fumigatus | F11628 | Clinical (Liverpool, UK) | Azole^{R} | Cyp51A mut. | Keller Laboratory |
| | | | | G138C | |
| A. fumigatus | F16216 | Clinical (Northampton, UK) | Azole^{R} | Cyp51A mut. L98H + | Keller Laboratory |
| | | | | TR | |
| A. terreus | NIH2624 | | — | | Keller Laboratory |

### Efficacy Against Yeast:

To evaluate the antifungal activity of MM-TM, DM-TM and NM against six species of yeast, across three different genera, we used the CLSI M27-A3 broth microdilution method (Table 51). Polymers were evaluated by measuring minimum inhibitory concentrations (MIC₁₀₀). For comparison, we also evaluated the commonly used antifungal drugs fluconazole (FLC), in terms of MIC₅₀ (the minimum inhibitory concentration to halt 50% of growth, as per the M27-A3 standard) and Amphotericin B (AmB, MIC₁₀₀), which is potent but toxic. As expected, all of the yeasts tested were sensitive to all three of the nylon-3 polymers, including those strains resistant to azoles. The polymers were particularly effective against Cryptococcus spp. (neoformans and amylolentus), exhibiting MIC₁₀₀ values from 2-4 μg/mL, which is comparable to or better than concentrations of fluconazole required to halt only 50% of fungal growth. Candida spp. were also susceptible to the polymers, with MIC₁₀₀ values for C. albicans and C. krusei generally ranging from 4-16 μg/mL. The polymers were also active against C. auris. The two strains of C. auris tested both showed sensitivity (MIC₁₀₀ 4-16 μg/mL) to all three polymers, with particular sensitivity to NM.

TABLE 51
MIC results for MM-TM, DM-TM and NM against yeast

| | | | MIC₁₀₀, μg/mL^{b} | | | |
|---|---|---|---|---|---|---|
| | | | | | | FLU |
| Species^{a} | Resistance | Isolate No. | NM | MM:TM | DM:TM | (MIC₅₀)^{c,d} |
| Saccharomyces | | W303 | 2 | 4 | 8 | <1 |
| cerevisiae | | | | | | |
| Cryptococcus | | CN1 | 4 | 4 | 4 | 4 |
| neoformans | | | | | | |
| Cryptococcus | FLU | CN2 | 4 | 4 | 4 | >64 |
| neoformans | | | | | | |
| Cryptococcus | FLU | CN3 | 4 | 4 | 4 | >64 |
| neoformans | | | | | | |
| Cryptococcus | | CBS 6273 | 4 | 4 | 2 | 16 |
| amolyentous | | | | | | |
| Cryptococcus | | CBS 6039 | 4 | 4 | 4 | 16 |
| amolyentous | | | | | | |
| Candida albicans | | ATCC 90028 | 4 | 4 | 4 | 1 |
| Candida albicans | | SC5314 | 8 | 16 | 16 | 0.25 |
| Candida albicans | Azole | CA3 | 8 | 16 | 64 | >64 |
| Candida krusei | | QC | 4 | 16 | 8 | 32 |
| Candida auris | | B11220 | 4 | 8 | 8 | 4 |
| Candida auris | AmB^{e} | C54039 | 8 | 16 | 16 | 16 |

^{a}Candida lusitaniae was tested and published¹¹ previously for MIC activity with MM-TM and shown to have and MIC₁₀₀ of 5 μg/mL.
^{b} MIC₁₀₀, Minimum inhibitory concentration resulting in 100% reduction in growth.
^{c}FLU, fluconazole.
^{d} MIC₅₀, Minimum inhibitory concentration resulting in 50% reduction in growth.
^{e}AmB = amphotericin

### Efficacy Against Dimorphic Fungi:

To determine polymer activities against dimorphic fungi, the CLSI M38-A2 broth microdilution method was used to test all three polymers against Coccidioides, Blastomyces, and Histoplasma. Each of these dimorphic fungi represents a significant health threat for humans, and the dimorphism of these fungi is an important feature for their pathogenicity. Activity was assessed based on 80% inhibition of growth (MIC_{K} as per the M38-A2 standard); the positive control for these studies was voriconazole (VOR) (Table 52). MM-TM, DM-TM, and NM have MIC₈₀ values <4 μg/mL for all strains of H. capsulatum, B. dermatitidis and Coccidioides spps. assayed.

TABLE II
MIC results for MM-TM, DM-TM and NM against
dimorphic fungi

| | | MIC₈₀, μg/mL^{a} | | | |
|---|---|---|---|---|---|
| | Isolate | | MM- | DM- | |
| Species | No. | NM | TM | TM | VOR^{b} |
| Coccidioides sp. | Cocci 1 | 1 | 2 | 2 | 0.25 |
| Coccidioides sp. | Cocci 2 | 1 | 2 | 2 | 0.125 |
| Coccidioides sp. | Cocci 3 | 0.5 | 1 | 1 | 0.06 |
| Coccidioides sp. | Cocci 5 | 2 | 2 | 1 | 0.125 |
| Coccidioides sp. | Cocci 6 | 1 | 1 | 1 | 0.25 |
| Coccidioides sp. | Cocci 7 | 1 | 1 | 1 | 0.125 |
| Coccidioides sp. | Cocci 8 | 1 | 1 | 1 | 0.125 |
| Coccidioides sp. | Cocci 9 | 1 | 2 | 2 | 1 |
| Coccidioides sp. | Cocci 10 | 1 | 1 | 1 | 0.06 |
| Blastomyces | BD1 | 1 | 2 | 1 | ≤0.03 |
| dermatitidis | | | | | |
| Blastomyces | BD2 | 1 | 2 | 1 | ≤3.03 |
| dermatitidis | | | | | |
| Blastomyces | BD3 | 1 | 2 | 1 | 0.5 |
| dermatitidis | | | | | |
| Histoplasma | HC1 | 4 | 2 | 1 | ≤0.03 |
| capsulatum | | | | | |
| Histoplasma | HC2 | 1 | 1 | 0.5 | 0.25 |
| capsulatum | | | | | |
| Histoplasma | HC3 | 4 | 2 | 2 | 0.125 |
| capsulatum | | | | | |
| Histoplasma | HC4 | 2 | 2 | 1 | 0.06 |
| capsulatum | | | | | |
| Histoplasma | HC5 | 2 | 2 | 1 | 0.25 |
| capsulatum | | | | | |
| Histoplasma | HC6 | 4 | 2 | 1 | 0.125 |
| capsulatum | | | | | |
| Histoplasma | HC7 | 2 | 2 | 1 | 0.5 |
| capsulatum | | | | | |
| Histoplasma | HC8 | 2 | 1 | 1 | 0.25 |
| capsulatum | | | | | |
| Histoplasma | HC9 | 4 | 2 | 2 | 0.06 |
| capsulatum | | | | | |
| Histoplasma | HC10 | 4 | 2 | 2 | 0.06 |
| capsulatum | | | | | |

^{a}Minimum inhibitory concentration required to halt 80% of growth (MIC₈₀).
^{b}VOR, voriconazole

### Efficacy Against Pneumocystis:

Antifungal activity of the three polymers was assessed against cryopreserved and characterized Pneumocystis spp. P. carinii is the causal agent of one of the AIDS-defining diseases, pneumocystis pneumonia (PCP). Fungal viability in the presence of each polymer was measured using an ATP production assay (ATP-lite M assay) after 24, 48, and 72 hours of polymer exposure. Calculating percent ATP reduction for all samples allowed us to determine 50% inhibitory concentrations (IC₅₀) (Table III). MM-TM and DM-TM exhibited moderate activity, with 72 hour IC₅₀ values ranging from 2 to 5 μg/mL. The homopolymer, NM, was less active against P. murina in comparison to P. carinii, with 72 hour IC₅₀ values of 15 and 3 μg/mL, respectively. Trimethoprim/sulfamethoxazole is the most effective clinical therapy for PCP; a three-week course of high dosages is required. This treatment regimen is associated with toxic side effects and high levels of antibiotic resistance. Given that the proposed mode of action of nylon-3 polymers involves rapid membrane disruption, similar to the mechanism ascribed to natural HDPs, the development of resistance to nylon-3 polymers is likely to be rare. Therefore, these polymers are useful as active ingredients in pharmaceutical compositions for anti-Pneumocystis therapy.

TABLE 53
IC₅₀ results for MM-TM, DM-TM, and NM against Pneumocystis

| | IC₅₀, μg/mL^{a} | | | |
|---|---|---|---|---|
| | | MM- | DM- | Activity Scale of Day 3 |
| Species | NM | TM | TM | IC₅₀ values |
| Pneumocystis | 3.4 | 4.5 | 4.8 | Moderate 1.0 to 9.99 μg/mL |
| carinii | | | | |
| Pneumocystis | 15 | 3.8 | 2.3 | Slight 10.0 to 49.9 μg/mL |
| murina | | | | |

^{a}Fifty percent inhibitory concentration (IC₅₀)

### Aspergillus Species:

We assessed the antifungal activity of MM-TM, DM-TM, and NM against 18 different species within the genus Aspergillus. For 6 of the 18 Aspergillus species examined, none of the 3 polymers caused any decrease in hyphal growth relative to a no-treatment control (Table 54, lines 1-6). Three of these species, A. fumigatus, A. flavus and A. terreus, are considered to be the most pathogenic species of the Aspergillus genus, suggesting that some aspect(s) corresponding to increased pathogenicity may be related to increased resistance to nylon-3 polymers. The remaining 12 species of Aspergillus evaluated showed low levels of growth inhibition, with MIC₁₀₀ ranging from 8 to >64 μg/mL.

TABLE 54
MIC results for MM-TM, DM-TM, and NM against species of
the Aspergillus genus

| | | MIC₁₀₀, μg/mL | | | |
|---|---|---|---|---|---|
| Species | Isolate No. | NM | MM-TM | DM-TM | ITRA |
| Aspergillus | CEA10 | >64 | >64 | >64 | <1 |
| fumigatus | | | | | |
| Aspergillus flavus | NRRL3357 | >64 | >64 | >64 | <1 |
| Aspergillus oryzae | Rib40 | >64 | >64 | >64 | 1 |
| Aspergillus terreus | NCCB | >64 | >64 | >64 | <1 |
| | IH2626 | | | | |
| Aspergillus | Su-1 | >64 | >64 | >64 | <1 |
| parasiticus | | | | | |
| Neosartorya fischeri | CBS | >64 | >64 | >64 | >8 |
| | 544.65 | | | | |
| Aspergillus nidulans | FGSCA4 | 16 | 8 | 8 | <1 |
| Aspergillus | CBS | >64 | >64 | 8 | <1 |
| aculeatus | 172.66 | | | | |
| Aspergillus | DT0115-B6 | 64 | 64 | 64 | <1 |
| carbonarius | | | | | |
| Aspergillus wentii | DT0136-E9 | >64 | 64 | 32 | <1 |
| Aspergillus sydowii | CBS | >64 | 64 | 64 | 2 |
| | 593.65 | | | | |
| Aspergillus foetidus | CBS | >64 | 64 | 32 | 2 |
| | 106.47 | | | | |
| Aspergillus zonatus | CBS | 16 | 8 | 8 | <1 |
| | 506.65 | | | | |
| Aspergillus niger | CBS 113.46 | 64 | 32 | 32 | <1 |
| Aspergillus glaucus | CBS | >64 | 64 | 32 | 2 |
| | 516.65 | | | | |
| Aspergillus | CBS | 64 | 48 | 48 | 64 |
| brasiliensis | 101740 | | | | |
| Aspergillus clavatus | CBS | >64 | >64 | >64 | 64 |
| | 513.65 | | | | |
| Aspergillus | CBS | 16 | 16 | 8 | 1 |
| versicolor | 795.97 | | | | |

Note, lines 1-6 (bold) indicate that no reduction in growth was observed after incubation with nylon-3 polymer. Lines 7-18 (no bold) indicate that some reduction in growth was noted in response to MM-TM, DM-TM, and NM₂₀, even when the MICs₁₀₀ were >64 μg/mL.
^{a}MIC₁₀₀, Minimum inhibitory concentration resulting in 100% reduction in growth.
^{b}ITRA, itraconazole

### Non-Aspergillus Filamentous Fungi:

Based on the observation of generally enhanced resistance of Aspergillus spp. towards the polymers, we hypothesized that nylon-3 polymers are intrinsically less effective against filamentous fungi relative to yeasts. Some studies have shown that fungal hyphae are more resistant to antifungal agents than are yeast, possibly through establishment of biofilms, which require higher effective concentrations of compounds for inhibition of growth. To test the hypothesis that filamentous fungi are less susceptible relative to yeasts, we assessed MM-TM, DM-TM, and NM activity against seven genera of filamentous fungi, following the CLSI M38-A2 methodology (conidia in liquid culture). Surprisingly, MM-TM, DM-TM, and NM were very active against phylogenetically diverse filamentous fungi, with MIC₁₀₀ values of 4-8 μg/mL. The polymers were much more effective against the emerging pathogen Paecilomyces variotii and Fusarium oxysporum isolates (along with A. flavus, a serious agent of keratitis) than they were against Aspergillus spp. Notably, all three nylon-3 polymers were active against Scedosporium spp., both apoiospermum and prolificans. The latter species is an emerging fungal pathogen of both immunocompetent and immunocompromised individuals that is intrinsically resistant to most antifungal drugs (VOR MIC₁₀₀>16 μg/mL); S. prolificans infections are often fatal.

The nylon-3 polymers were active against Rhizopus arrhizus, one of the causative agents of mucormycosis, which is a life-threatening disease in both immunocompetent and immunocompromised people. Depending on the pre-disease status of the patient and route of infection, mucormycosis may present in pulmonary, rhino-orbital-cerebral, cutaneous, gastrointestinal, or disseminated forms. Treatment of mucormycosis necessitates the use of AmB often after surgical debridement of necrotic tissues. Even with rigorous treatment regimes, mortality rates are high (>40%), and AmB toxicity is problematic for patients. The sensitivity of R. arrhizus to nylon-3 polymers indicates the present compounds are pharmacologically active to inhibit a challenging and deadly fungal disease for which current treatment options are highly limited.

TABLE 55
MIC results for MM-TM, DM-TM, and NM against filamentous fungi

| | | MIC₁₀₀, μg/mL^{a} | | | | | | |
|---|---|---|---|---|---|---|---|---|
| | Isolate | | | | | | | FLU |
| Species | No. | NM | MM-TM | DM-TM | POS^{b} | VOR^{c} | ITRA^{d} | (MIC₅₀)^{e, f} |
| Talaromyces | | 64 | 32 | 8 | — | — | 0.125 | — |
| marneffei | | | | | | | | |
| Penicillium | | >64 | >64 | 64 | — | — | 1 | — |
| expansum | | | | | | | | |
| Paecilomyces | QC | 4 | 8 | 4 | — | 0.125 | — | — |
| variotii | | | | | | | | |
| Fusarium | FO1 | 4 | 8 | 8 | — | 4 | — | — |
| oxysporum | | | | | | | | |
| Fusarium | FO2 | 8 | 8 | 8 | — | 4 | — | — |
| oxysporum | | | | | | | | |
| Fusarium | FO3 | 4 | 8 | 4 | — | >16 | — | — |
| oxysporum | | | | | | | | |
| Scedosporium | SA1 | 2 | 8 | 4 | — | 1 | — | — |
| apiopsermum | | | | | | | | |
| Scedosporium | SA2 | 4 | 4 | 4 | — | 1 | — | — |
| apiopsermum | | | | | | | | |
| Scedosporium | LP1 | 4 | 4 | 4 | — | >16 | — | — |
| prolificans | | | | | | | | |
| Rhizopus arrhizus | RA1 | 16 | 8 | 4 | 0.5 | — | — | — |
| Rhizopus arrhizus | RA2 | 8 | 8 | 8 | 0.5 | — | — | — |
| Rhizopus arrhizus | RA3 | 8 | 8 | 8 | 0.5 | — | — | — |
| Filobasidiella | CBS | >64 | >64 | >64 | — | — | — | 64 |
| depauperata | 7855 | | | | | | | |

^{a}MIC₁₀₀, Minimum inhibitory concentration resulting in 100% reduction in growth.
^{b}POS, posaconazole
^{c}VOR, voriconazole
^{d}ITRA, itraconazole
^{e}FLU, fluconazole.
^{f} MIC₅₀, Minimum inhibitory concentration resulting in 50% reduction in growth.

### Dermatophytes:

The final group of fungi tested for sensitivity to nylon-3 polymers were dermatophytes. In contrast to the other filamentous fungi tested, three of these isolates were evaluated following a modified version of the CLSI M38-A methodology (using hyphal fragments in liquid culture, because of difficulties in obtaining conidia). For the human pathogens Trichophyton tonsurans, Trichophyton rubrum, and Microsporum canis, MIC₁₀₀ values were determined as the concentration of the agent required to halt hyphal growth, monitored as an increase in OD₆₀₀, after five days of incubation at 29° C. in RPMI-1640. The MIC₁₀₀ of the bat pathogen Pseudogymnoascus destructans was similarly evaluated after seven days of incubation of conidia at 12° C. in RPMI-1640. Mixed efficacy of the nylon-3 polymers was observed across the four species of dermatophytes tested. The highest levels of antifungal activity were observed against P. destructans and T. tonsurans (2-16 μg/mL), and the lowest levels were observed against T. rubrum and M. canis (16-64 μg/mL) (Table 56). The activity against P. destructans, the causative agent of White Nose syndrome in bats, and just one of many emerging fungal pathogens threatening wildlife is encouraging because there are few options at present for preventing the spread of this devastating pathogen. The facility with which nylon-3 polymers can be modified could provide opportunities for the development of topical agents with high specificity for particular fungi.

TABLE 56
MIC results for MM-TM, DM-TM, and NM against
dermatophytes

| | | MIC₁₀₀, μg/mL^{a} | | | |
|---|---|---|---|---|---|
| | | | MM- | DM- | |
| Species | Isolate No. | NM | TM | TM | ITRA^{b} |
| Trichophyton rubrum | ATCC 28188 | >64 | 64 | 32 | 1 |
| Microsporum canis | UW10 | 64 | 32-64 | 16 | 1 |
| Trichophyton tonsurans | CBS 112818 | 32 | 16 | 8-16 | 0.13 |
| Pseudogymnoascus | ATC MYA- | 4-8 | 2-4 | 2-4 | 0.13 |
| destructans | 4855 | | | | |

^{a}MIC₁₀₀, Minimum inhibitory concentration resulting in 100% reduction in growth.
^{b}ITRA, itraconazole

### Mammalian Toxicity:

Having observed activity of the three nylon-3 polymers against a broad swath of phylogenetically diverse fungi, we performed two mammalian cytotoxicity studies. Adenocacincomic human alveolar basal epithelial cells, A549, and the murine T-cell hybridoma L2 cell line were treated with individual polymers for 72 hours and then evaluated for ATP production as a measure of viability. MM-TM, DM-TM and NM were found to be non-toxic (IC₅₀>100 μg/mL) against the A549 cell line. More variability in toxicity was observed against the L2 cell line, in which trends followed those of previously published nylon-3 toxicity data. Against the L2 cell line, the cationic homopolymer, NM, was nontoxic (IC₅₀>100 μg/mL), while the more hydrophobic MM-TM and DM-TM copolymers exhibited mild to moderate toxicity against this cell line (IC₅₀=22 and 7 μg/mL respectively).

TABLE 57
Mammalian cell toxicity of nylon-3 polymers

| | IC₅₀, μg/mL^{a} | | |
|---|---|---|---|
| | | MM- | DM- |
| Cell line | NM | TM | TM |
| A549 | >100 | >100 | >100 |
| L2 | >100 | 21.82 | 7.29 |

^{a}Fifty percent inhibitory concentration (IC₅₀)

The data disclosed herein show that the nylon-3 polymers MM-TM, DM-TM and NM are effective against a surprisingly broad spectrum of fungi, with only low to moderate toxicity toward mammalian cells. The human mycobiome is vast, with pathogenic species found in three highly diverged phyla, the Zygomycetes, Ascomycetes and Basidiomycetes. Here we were able to assess sensitivity of 16 pathogenic genera towards the nylon-3 chemotype, based on measurements with 40 species and 70 isolates.

What is claimed is:

1. A method of inhibiting fungal growth, the method comprising:
contacting fungi with a composition comprising a nylon-3 copolymer having a formula: 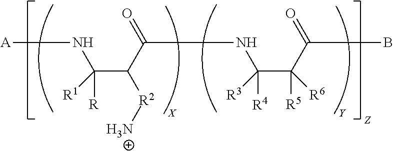
or a salt thereof,
wherein:
each R is independently hydrogen or C₁-C₆-alkyl;
each R¹, R³, R⁴, R⁵, and R⁶ are each independently selected from the group consisting of hydrogen or substituted or unsubstituted C₁-C₆-alkyl;
each R² is C₁-C₆-alkylene;
“A” is hydrogen or an amino-protecting group;
“B” is hydroxyl or a carboxy-protecting group; and
“X,” “Y,” and “Z” are positive numbers.

2. The method of claim 1, wherein the nylon-3 copolymer is a random copolymer.

3. The method of claim 1, wherein the nylon-3 copolymer is a block copolymer.

4. The method of claim 1, wherein:
R¹, R³, R⁴, R⁵, and R⁶ are each methyl; and
R² is methylene.

5. The method of claim 1, wherein:
“A” is 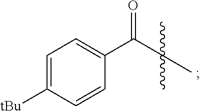 [SMILES: *C(=O)c1ccc(C(C)(C)C)cc1]
and
“B” is 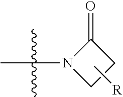
wherein R is hydrogen or C₁-C₆-alkyl.

6. The method of claim 1, wherein X is a number between 0.1 and 0.9, Y is a number between 0.1 and 0.9, and Z is a number between 5 and 100.

7. The method of claim 1, wherein X is a number between 0.1 and 0.9, Y is a number between 0.1 and 0.9, and Z is a number between 10 and 50.

8. The method of claim 1, wherein X is a number between 0.1 and 0.9, Y is a number between 0.1 and 0.9, and Z is a number between 10 and 20.

9. The method of claim 1, comprising contacting the composition to a fungi of the genera Aspergillus, Candida, Cryptococcus, and/or Fusarium.

10. A method of inhibiting fungal infections in mammals, the method comprising:
administering to a mammalian subject in need thereof a fungal growth-inhibiting amount of a nylon-3 copolymer having a formula: 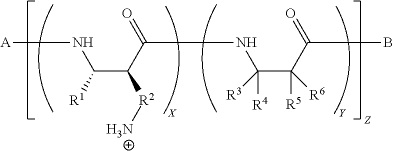
or a salt thereof,
wherein:
R¹, R³, R⁴, R⁵, and R⁶ are each independently selected from the group consisting of hydrogen or substituted or unsubstituted C₁-C₆-alkyl;
R² is C₁-C₆-alkylene;
“A” is hydrogen or an amino-protecting group;
“B” is hydroxyl or a carboxy-protecting group; and
“X,” “Y,” and “Z” are positive numbers.

11. The method of claim 10, which is a method treating a fungal infection by a fungus within the genera Aspergillus, Candida, Cryptococcus, and/or Fusarium.

12. The method of claim 10, wherein the nylon-3 copolymer is a random copolymer.

13. The method of claim 10, wherein the nylon-3 copolymer is a block copolymer.

14. The method of claim 10, wherein:
R¹, R³, R⁴, R⁵, and R⁶ are each methyl; and
R² is methylene.

15. The method of claim 10, wherein:
“A” is 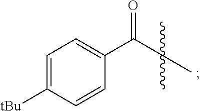 [SMILES: *C(=O)c1ccc(C(C)(C)C)cc1]
and
“B” is 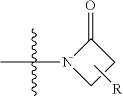
wherein R is hydrogen or C₁-C₆-alkyl.

16. The method of claim 10, wherein X is a number between 0.1 and 0.9, Y is a number between 0.1 and 0.9, and Z is a number between 5 and 100.

17. The method of claim 10, wherein X is a number between 0.1 and 0.9, Y is a number between 0.1 and 0.9, and Z is a number between 10 and 50.

18. The method of claim 10, wherein X is a number between 0.1 and 0.9, Y is a number between 0.1 and 0.9, and Z is a number between 10 and 20.

19. A pharmaceutical composition comprising:
a fungal growth-inhibiting amount of a nylon-3 copolymer having a formula: 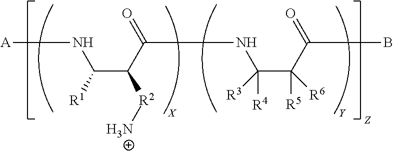
or a pharmaceutically suitable salt thereof,
wherein:
R¹, R³, R⁴, R⁵, and R⁶ are each independently selected from the group consisting of hydrogen or substituted or unsubstituted C₁-C₆-alkyl;
R² is C₁-C₆-alkylene;
“A” is hydrogen or an amino-protecting group;
“B” is hydroxyl or a carboxy-protecting group; and
“X,” “Y,” and “Z” are positive numbers;
in combination with a pharmaceutically suitable carrier.

20. The composition of claim 19, wherein the nylon-3 copolymer is a random copolymer.

21. The composition of claim 19, wherein the nylon-3 copolymer is a block copolymer.

22. The composition of claim 19, wherein:
R¹, R³, R⁴, R⁵, and R⁶ are each methyl; and
R² is methylene.

23. The composition of claim 19, wherein:
“A” is 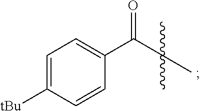 [SMILES: *C(=O)c1ccc(C(C)(C)C)cc1]
and
“B” is 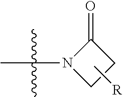
wherein R is hydrogen or C₁-C₆-alkyl.

24. The composition of claim 19, wherein X is a number between 0.1 and 0.9, Y is a number between 0.1 and 0.9, and Z is a number between 5 and 100.

25. The composition of claim 19, wherein X is a number between 0.1 and 0.9, Y is a number between 0.1 and 0.9, and Z is a number between 10 and 50.

26. The composition of claim 19, wherein X is a number between 0.1 and 0.9, Y is a number between 0.1 and 0.9, and Z is a number between 10 and 20.
